(19) United States
(12) Patent Application Publication
(10) Pub. No.: US 2016/0010145 A1
(43) Pub. Date: Jan. 14, 2016

(54) DETECTION OF RNA WITH SUBSTITUTED UNSYMMETRICAL CYANINE DYES

(71) Applicant: LIFE TECHNOLOGIES CORPORATION, Carlsbad, CA (US)

(72) Inventors: Jason DALLWIG, Eugene, OR (US); Kyle GEE, Springfield, OR (US); Nancy AHNERT, Eugene, OR (US); Hee Chol KANG, Eugene, OR (US); Jongtae YANG, Carlsbad, CA (US)

(21) Appl. No.: 14/649,370

(22) PCT Filed: Dec. 6, 2013
(86) PCT No.: PCT/US13/73548

§ 371 Date: Jun. 3, 2015

Related U.S. Application Data
(60) Provisional application No. 61/734,862, filed on Dec. 7, 2012.
(60) Provisional application No. 61/872,276, filed on Aug. 30, 2013.

(51) Int. Cl. C12Q 1/68; C09B 23/00

(52) U.S. Cl. CPC C12Q 1/6825; C09B 23/00

(57) ABSTRACT

Substituted unsymmetrical cyanine dyes are provided herein, including kits, assays systems and methods for their use in the detection of RNA compounds (e.g., miRNA).

### RELATED APPLICATIONS

This application claims the benefit of U.S. Provisional Application Nos. 61/872,276, filed Aug. 30, 2013 and 61/734,862, filed Dec. 7, 2012, which disclosures are herein incorporated by reference in their entirety.

### TECHNICAL FIELD

Substituted unsymmetrical cyanine dyes are provided that are useful in the detection of RNA molecules (e.g., microRNA); also provided are compositions, kits and assay systems that include such dyes, as well as methods for their use.

### BACKGROUND

MicroRNAs (miRNAs) are small, highly conserved RNA molecules that, like small interfering RNAs (siRNAs), modulate the expression of their cognate mRNA molecules by base-pairing with them and preventing expression through a variety of mechanisms. miRNAs have been shown to act as key regulators of development, cell proliferation, differentiation, and the cell cycle.

Although various nucleic acid binding dyes are known, there remains a need to develop materials and methods for detecting nucleic acid molecules, in particular, small single-stranded RNA molecules, such as microRNA.

### SUMMARY

Provided herein are substituted unsymmetrical cyanine dyes, kits and compositions including such dyes, as well as methods using such dyes for detecting RNA molecules. The disclosed dyes and compositions can be used advantageously for the detection of short and/or single-stranded RNA molecules. It was surprisingly found that the dye compounds and the methods disclosed herein can be used to detect short, single-stranded RNA molecules that are usually not detectable by standard fluorescent methods. Furthermore, the methods provided herein can detect very low concentrations of short RNA molecules. It was also surprisingly found that the compositions and methods provided herein are more specific for short RNA molecules than for larger RNA molecules. The compositions and methods provided herein overcome many of the disadvantages associated with conventional double-stranded nucleic acid binding dyes and provide new opportunities for the use in the analysis and quantification of RNA.

In one aspect, a method of detecting the presence of single-stranded RNA (ssRNA) in a sample is provided, the method includes:
(a) combining a sample suspected of containing ssRNA, wherein the ssRNA comprises 25 or fewer nucleotides, with a substituted, unsymmetrical cyanine dye compound;
(b) incubating the sample for a time sufficient for the dye compound to combine with the ssRNA in the sample, if present, to form a dye-ssRNA complex that emits a detectable fluorescent signal; and
(c) detecting the emission of the fluorescent signal, wherein the detectable fluorescent signal is indicative of the presence of a dye-ssRNA complex.

In certain embodiments, the ssRNA comprises 20-25 nucleotides. In certain embodiments, the ssRNA comprises 10-20 nucleotides. In certain embodiments, the ssRNA is naturally occurring. In certain embodiments, the ssRNA comprises a chemical modification to at least one sugar, phosphate or base group. In certain embodiments, the ssRNA comprises a chemical group other than a hydroxyl or phosphate at its 5′ termini. In certain embodiments, the ssRNA is microRNA (miRNA), small nuclear RNA (snRNA), small nucleolar RNA (snoRNA), tiny non-coding RNA (tncRNA), small modulatory RNA (smRNA), or piwi-interacting RNA (piRNA). In certain embodiments, the ssRNA is miRNA.

The methods provided herein can detect as little as 0.5 ng ssRNA in a sample even in the presence of larger RNA, such as mRNA or rRNA. In certain embodiments, the method further comprises detecting ssRNA in a concentration of 0.5 ng/mL or greater. In certain embodiments, the method further comprises detecting ssRNA in a concentration of 5 ng/mL or greater. In certain embodiments, the method further comprises detecting ssRNA in a concentration of 10 ng/mL or greater. In certain embodiments, the method further comprises detecting ssRNA in a concentration of 25 ng/mL or greater. In certain embodiments, the method further comprises detecting ssRNA in a concentration of about 5 ng/mL to about 1000 ng/mL. In certain embodiments, the method further comprises detecting ssRNA in a concentration of about 25 ng/mL to about 1000 ng/mL. In certain embodiments, the method further comprises detecting ssRNA in a concentration of about 50 ng/mL to about 1000 ng/mL. In certain embodiments, the sample can include ssRNA in a concentration of about 5 ng/mL to about 500 ng/mL. In certain embodiments, the sample can include ssRNA in a concentration of about 25 ng/mL to about 500 ng/mL. In certain embodiments, the sample can include ssRNA in a concentration of about 50 ng/mL to about 100 ng/mL.

In certain embodiments, the fluorescence signal is stable for at least 30 minutes at room temperature. In certain embodiments, the fluorescence signal is stable for at least 3 hours at room temperature.

In another aspect, a method of detecting the presence of single-stranded RNA (ssRNA) in a sample is provided, the method includes:
(a) combining a sample suspected of containing ssRNA, wherein the ssRNA comprises 25 or fewer nucleotides, with a substituted, unsymmetrical cyanine dye compound of Formula I:

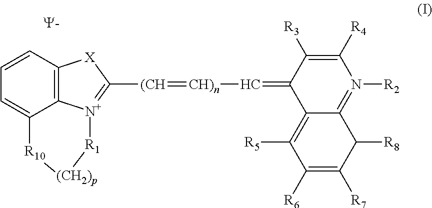

wherein
X is O or S;
n is 0, 1 or 2;
p is 0, 1 or 2;
Ψ is a biologically compatible counterion;
R₁ and R₁₀, which can be the same or different, are independently H; or a substituted or unsubstituted alkyl group, wherein the substitution on the alkyl group, if present, is a carboxyl or (alkyl)ammonium group;
R₂ is a substituted or unsubstituted alkyl, naphthyl, phenyl, thienyl, or cycloalkyl having 3-8 carbons, wherein the substitution on R₂, if present, is a carboxyl or carboxamide group;
R₃ is H; or an alkyl, alkenyl, polyalkenyl, alkynyl or polyalkynyl group; or a halogen; or a substituted or unsubstituted aryl or heteroaryl; or a substituted or unsubstituted cycloalkyl having 3-10 carbons; or —OR₁₁, —SR₁₁, —(NR₉R₁₁); or —OSO₂R₁₉; or a TAIL;
wherein R₉ and R₁₁, which can be the same or different, are independently H; or substituted or unsubstituted alkyl groups; or alkyl groups substituted with one or more alkylammonium groups; or 1-2 alicyclic or aromatic rings; or R₉ and R₁₁ taken in combination are —(CH₂)₄— or —(CH₂)₅— to give a 5 or 6 membered ring; and where R₁₉ is alkyl, or perfluoroalkyl, or aryl;
R₄ is an alkyl, alkenyl, polyalkenyl, alkynyl or polyalkynyl group; or a halogen; or a substituted or unsubstituted aryl or heteroaryl; or a substituted or unsubstituted cycloalkyl having 3-10 carbons; or —OR₁₁, —SR₁₁, —(NR₉R₁₁); or —OSO₂R₁₉; or a TAIL;
R₅, R₆, R₇ and R₈, which can be the same or different, are independently H; or an alkyl, alkenyl, polyalkenyl, alkynyl or polyalkynyl group; or a halogen; or a substituted or unsubstituted aryl or heteroaryl; or a substituted or unsubstituted cycloalkyl having 3-8 carbons; or a TAIL; or —OR₁₁, —SR₁₁, or —(NR₉R₁₁);
TAIL is a heteroatom-containing moiety having the formula LINK-SPACER-CAP;
wherein
LINK is a single covalent bond, —O—, —S—, or —NR₂₀—; where R₂₀ is H, a linear or branched alkyl having 1-8 carbons, or R₂₀ is -SPACER′-CAP′;
SPACER and SPACER′, which can be the same or different, are covalent linkages, linear or branched, cyclic or heterocyclic, saturated or unsaturated, each having 1-16 nonhydrogen atoms selected from the group consisting of C, N, O and S, such that the linkage begins and ends with a carbon atom, and contains any combination of ether, thioether, amine, ester, amide, or aliphatic, oleic or aromatic carbon-carbon bonds, or aromatic carbon-nitrogen or nitrogen-nitrogen bonds; wherein all heteroatoms in the linear backbone of SPACER are separated by at least two carbon atoms;
CAP and CAP′, which can be the same or different, are —OR₂₁, —SR₂₁, —NR₂₁R₂₂, or —N⁺R₂₁R₂₂R₂₃Ψ⁻;
wherein
R₂₁, R₂₂, and R₂₃ are independently H, or a linear or branched alkyl or cycloalkyl having 1-8 carbons, optionally further substituted by halogen, hydroxy, alkoxy having 1-8 carbons, carboxyalkyl having 1-8 carbons, or phenyl, where phenyl is optionally further substituted by halogen, hydroxy, alkoxy having 1-8 carbons, aminoalkyl having 1-8 carbons, or carboxyalkyl having 1-8 carbons; or, one or more of R₂₁, R₂₂, and R₂₃, taken in combination with SPACER or SPACER′ or R₂₀ forms a 5- or 6-membered aromatic, heteroaromatic, alicyclic or heteroalicyclic ring, the heteroatoms selected from O, N or S; where Ψ⁻ is a biologically compatible counterion; or
CAP and CAP′ are independently

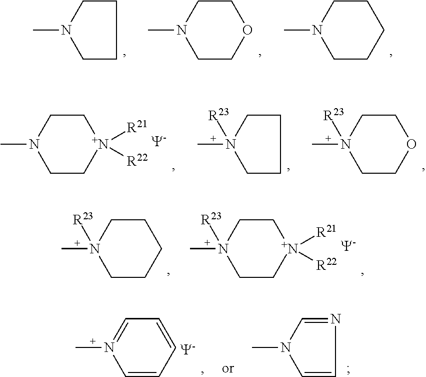

where R₂₁, R₂₂, R₂₃ and Ψ⁻ are as defined previously; such that at least one of R₃, R₄, R₅, R₆, R₇, R₈ or R₁₀ is a TAIL; and, where more than one substituent is a TAIL, each TAIL is optionally the same or different;

or wherein at least one of R₁, R₅, R₆, R₇, R₈, R₉, R₁₀, R₁₁ or R₂₀, R₂₁, R₂₂ or R₂₃ is a water-soluble group (WSG);
(b) incubating the sample for a time sufficient for the dye compound to combine with the ssRNA in the sample, if present, to form a dye-ssRNA complex that emits a detectable fluorescent signal; and
(c) detecting the emission of the fluorescent signal, wherein the detectable fluorescent signal is indicative of the presence of a dye-ssRNA complex.

The water-soluble group can include a polymer (e.g., a polyalkylene oxide, carbohydrate and polypeptide). In certain embodiments, the water-soluble group comprises a polyethylene oxide. In certain embodiments, R₄ is a TAIL; R₂ is a substituted or unsubstituted phenyl; and/or at least one of R₅, R₆, R₇ and R₈ is —OR₁₁, —SR₁₁, or —NR₉R₁₁). In certain embodiments, n is 0. In certain embodiments, at least one of R₅, R₆, R₇ and R₈ is —OR₁₁. In certain embodiments, OR₁₁ is —OCH₃. In certain embodiments, R₇ is —OCH₃. In certain embodiments, R₁ is an unsubstituted alkyl and R₁₀ is H. In certain embodiments, R₁ is methyl and R₁₀ is H.

When CAP is —N⁺R₂₁R₂₂R₂₃Ψ⁻, the biologically compatible counter ion balances the positive charge present on the CAP nitrogen, which is a quaternary ammonium salt. As used herein, a substance that is biologically compatible is not toxic as used, and does not have a substantially deleterious effect on biomolecules. Examples of Ψ⁻ include, among others, chloride, bromide, iodide, sulfate, alkanesulfonate, arylsulfonate, phosphate, perchlorate, tetrafluoroboronate, tetraarylboride, nitrate, and anions of aromatic or aliphatic carboxylic acids. Preferred Ψ− counterions are bromide, chloride, iodide, perchlorate, and various sulfonates.

In certain embodiments, wherein if p is 0, and wherein R₁ and R₁₀, which can be the same or different, are independently H; or a substituted or unsubstituted alkyl group, wherein the substitution on the alkyl group, if present, is a carboxyl or (alkyl)ammonium group. In other embodiments, wherein if p is not 0, R₁ and R₁₀, which can be the same or different, are independently a substituted or unsubstituted alkyl group, wherein the substitution on the alkyl group, if present, is a carboxyl or (alkyl)ammonium group.

In certain embodiments, the dye compound of Formula I is selected from the group consisting of Compound 4, Compound 5, Compound 6, Compound 7, Compound 8, Compound 9, Compound 10, Compound 11, Compound 12, Compound 13, Compound 14, Compound 15, Compound 16, Compound 17, Compound 18, Compound 19, Compound 20, Compound 21, Compound 23, Compound 25, Compound 26, Compound 27, Compound 30, Compound 31, Compound 32, Compound 33, Compound 34, Compound 35, Compound 36, Compound 37, Compound 38, Compound 39, Compound 40, Compound 41, Compound 42, Compound 43, Compound 44, Compound 45, Compound 46, Compound 47, Compound 48, Compound 49, Compound 50, Compound 51, Compound 52, Compound 53, Compound 54, Compound 55, Compound 56, Compound 57, Compound 58, Compound 59, Compound 60, Compound 61, Compound 62, Compound 63, Compound 64, and Compound 65.

In certain embodiments, the ssRNA comprises 20-25 nucleotides. In certain embodiments, the ssRNA comprises 10-20 nucleotides. In certain embodiments, the ssRNA is naturally occurring. In certain embodiments, the ssRNA comprises a chemical modification to at least one sugar, phosphate or base group. In certain embodiments, the ssRNA comprises a chemical group other than a hydroxyl or phosphate at its 5′ termini. In certain embodiments, the ssRNA is microRNA (miRNA), small nuclear RNA (snRNA), small nucleolar RNA (snoRNA), tiny non-coding RNA (tncRNA), small modulatory RNA (smRNA), or piwi-interacting RNA (piRNA). In certain embodiments, the ssRNA is miRNA.

The methods provided herein can detect as little as 0.5 ng ssRNA in a sample even in the presence of larger RNA, such as mRNA or rRNA. In certain embodiments, the method further comprises detecting ssRNA in a concentration of 0.5 ng/mL or greater. In certain embodiments, the method further comprises detecting ssRNA in a concentration of 5 ng/mL or greater. In certain embodiments, the method further comprises detecting ssRNA in a concentration of 10 ng/mL or greater. In certain embodiments, the method further comprises detecting ssRNA in a concentration of 25 ng/mL or greater. In certain embodiments, the method further comprises detecting ssRNA in a concentration of about 5 ng/mL to about 1000 ng/mL. In certain embodiments, the method further comprises detecting ssRNA in a concentration of about 25 ng/mL to about 1000 ng/mL. In certain embodiments, the method further comprises detecting ssRNA in a concentration of about 50 ng/mL to about 1000 ng/mL. In certain embodiments, the sample can include ssRNA in a concentration of about 5 ng/mL to about 500 ng/mL. In certain embodiments, the sample can include ssRNA in a concentration of about 25 ng/mL to about 500 ng/mL. In certain embodiments, the sample can include ssRNA in a concentration of about 50 ng/mL to about 100 ng/mL.

In certain embodiments, the fluorescence signal is stable for at least 30 minutes at room temperature. In certain embodiments, the fluorescence signal is stable for at least 3 hours at room temperature.

In yet another aspect, a method of detecting single-stranded RNA (ssRNA) in a sample is provided, the method includes:
(a) combining a sample suspected of containing ssRNA, wherein the ssRNA comprises 25 or fewer nucleotides, with a substituted, unsymmetrical cyanine dye compound of the Formula II,

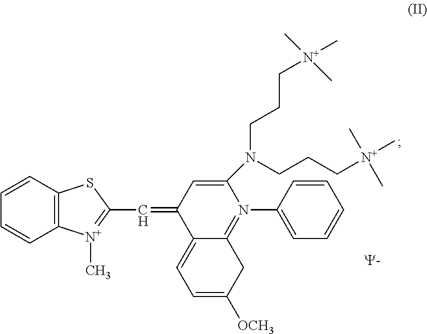

(b) incubating the sample for a time sufficient for the dye compound to combine with the ssRNA in the sample, if present, to form a dye-ssRNA complex that emits a detectable fluorescent signal; and
(c) detecting the emission of the fluorescent signal, wherein the detectable fluorescent signal is indicative of the presence of a dye-ssRNA complex.

Examples of Ψ⁻ include, among others, chloride, bromide, iodide, sulfate, alkanesulfonate, arylsulfonate, phosphate, perchlorate, tetrafluoroboronate, tetraarylboride, nitrate, and anions of aromatic or aliphatic carboxylic acids. Preferred ψ-counterions are bromide, chloride, iodide, perchlorate, and various sulfonates.

In certain embodiments, the ssRNA comprises 20-25 nucleotides. In certain embodiments, the ssRNA comprises 10-20 nucleotides. In certain embodiments, the ssRNA is naturally occurring. In certain embodiments, the ssRNA comprises a chemical modification to at least one sugar, phosphate or base group. In certain embodiments, the ssRNA comprises a chemical group other than a hydroxyl or phosphate at its 5′ termini. In certain embodiments, the ssRNA is microRNA (miRNA), small nuclear RNA (snRNA), small nucleolar RNA (snoRNA), tiny non-coding RNA (tncRNA), small modulatory RNA (smRNA), or piwi-interacting RNA (piRNA). In certain embodiments, the ssRNA is miRNA.

The methods provided herein can detect as little as 0.5 ng ssRNA in a sample even in the presence of larger RNA, such as mRNA or rRNA. In certain embodiments, the method further comprises detecting ssRNA in a concentration of 0.5 ng/mL or greater. In certain embodiments, the method further comprises detecting ssRNA in a concentration of 5 ng/mL or greater. In certain embodiments, the method further comprises detecting ssRNA in a concentration of 10 ng/mL or greater. In certain embodiments, the method further comprises detecting ssRNA in a concentration of 25 ng/mL or greater. In certain embodiments, the method further comprises detecting ssRNA in a concentration of about 5 ng/mL to about 1000 ng/mL. In certain embodiments, the method further comprises detecting ssRNA in a concentration of about 25 ng/mL to about 1000 ng/mL. In certain embodiments, the method further comprises detecting ssRNA in a concentration of about 50 ng/mL to about 1000 ng/mL. In certain embodiments, the sample can include ssRNA in a concentration of about 5 ng/mL to about 500 ng/mL. In certain embodiments, the sample can include ssRNA in a concentration of about 25 ng/mL to about 500 ng/mL. In certain embodiments, the sample can include ssRNA in a concentration of about 50 ng/mL to about 100 ng/mL.

In certain embodiments, the fluorescence signal is stable for at least 30 minutes at room temperature. In certain embodiments, the fluorescence signal is stable for at least 3 hours at room temperature.

In yet another aspect, a method of detecting single-stranded (ss)RNA in a sample is provided, the method includes:
(a) combining a sample suspected of containing ssRNA, wherein the ssRNA comprises 25 or fewer nucleotides, with a substituted, unsymmetrical cyanine dye compound, wherein the dye comprises a first heterocyclic ring system that is a substituted benzothiazolium, benzoxazolium, benzoselenazolium, benzimidazolium, or dialkylindolinium, wherein the first ring system is linked by a monomethine or trimethine bridging moiety attached at the 2-position of the first ring system to the 2- or 4-position of a second heterocyclic ring system that is a substituted quinolinium or that is linked by a monomethine bridging moiety attached to the 2-position of a second heterocyclic ring system that is a substituted pyridinium; wherein one or more substituents of the second ring system is a substituted or unsubstituted cyclohexyl, cyclohexenyl, morpholino, piperidinyl, naphthyl, phenyl, thienyl, benzothizolyl, furanyl, oxazolyl, benzoxazolyl or pyridinyl group;
(b) incubating the sample for a time sufficient for the dye compound to combine with the ssRNA in the sample, if present, to form a dye-ssRNA complex that emits a detectable fluorescent signal; and
(c) detecting the emission of the fluorescent signal, wherein the detectable fluorescent signal is indicative of the presence of a dye-ssRNA complex.

The methods provided herein can be used to detect any type of short RNA molecule. The short RNA molecule can be a single-stranded RNA (ssRNA) molecule. The ssRNA can be naturally occurring or can include a chemical modification to at least one sugar, phosphate or base group. For example, the ssRNA can include a chemical group other than a hydroxy or phosphate at its 5′ terminus. In certain embodiments, the ssRNA molecule can include 20-25 nucleotides. In other embodiments, the ssRNA molecule can include 10-20 nucleotides. In certain methods, the ssRNA is microRNA (miRNA). The ssRNA can be a small nuclear RNA (snRNA), small nucleolar RNA (snoRNA), tiny non-coding RNA (tncRNA), small modulatory RNA (smRNA) or piwi-interacting RNA (piRNA).

The methods provided herein can be used to detect extremely small quantities of RNA (e.g., ssRNA) in a sample. For example, the methods provided herein can detect as little as 0.5 ng ssRNA in a sample even in the presence of larger RNA such as rRNA or mRNA. In certain embodiments, the method further comprises detecting ssRNA in a concentration of 0.5 ng/mL or greater. In certain embodiments, the method further comprises detecting ssRNA in a concentration of 5 ng/mL or greater. In certain embodiments, the method further comprises detecting ssRNA in a concentration of 10 ng/mL or greater. In certain embodiments, the method further comprises detecting ssRNA in a concentration of 25 ng/mL or greater. In certain embodiments, the method further comprises detecting ssRNA in a concentration of about 5 ng/mL to about 1000 ng/mL. In certain embodiments, the method further comprises detecting ssRNA in a concentration of about 25 ng/mL to about 1000 ng/mL. In certain embodiments, the method further comprises detecting ssRNA in a concentration of about 50 ng/mL to about 1000 ng/mL. In certain embodiments, the sample can include ssRNA in a concentration of about 5 ng/mL to about 500 ng/mL. In certain embodiments, the sample can include ssRNA in a concentration of about 25 ng/mL to about 500 ng/mL. In certain embodiments, the sample can include ssRNA in a concentration of about 50 ng/mL to about 100 ng/mL.

In certain embodiments, the fluorescence signal is stable for at least 30 minutes at room temperature. In certain embodiments, the fluorescence signal is stable for at least 3 hours at room temperature.

In yet another aspect, a fluorescent complex is provided that includes ssRNA, wherein the ssRNA comprises 25 or fewer nucleotides, non-covalently bound to a dye of the Formula II:

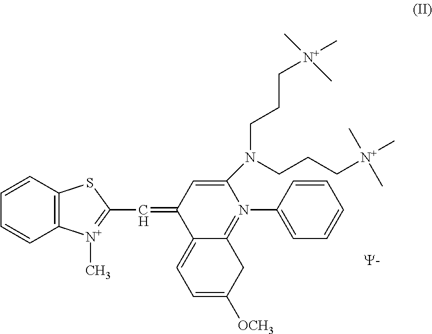

wherein the complex emits a detectable fluorescent signal.

Examples of Ψ⁻ include, among others, chloride, bromide, iodide, sulfate, alkanesulfonate, arylsulfonate, phosphate, perchlorate, tetrafluoroboronate, tetraarylboride, nitrate, and anions of aromatic or aliphatic carboxylic acids. Preferred ψ-counterions are bromide, chloride, iodide, perchlorate, and various sulfonates.

The short RNA molecule can be a single-stranded RNA (ssRNA) molecule. The ssRNA can be naturally occurring or can include a chemical modification to at least one sugar, phosphate or base group. For example, the ssRNA can include a chemical group other than a hydroxy or phosphate at its 5′ terminus. In certain embodiments, the ssRNA molecule can include 20-25 nucleotides. In other embodiments, the ssRNA molecule can include 10-20 nucleotides. In certain methods, the ssRNA is microRNA (miRNA). The ssRNA can be a small nuclear RNA (snRNA), small nucleolar RNA (snoRNA), tiny non-coding RNA (tncRNA), small modulatory RNA (smRNA) or piwi-interacting RNA (piRNA).

In yet another aspect, a fluorescent complex is produced that includes ssRNA, wherein the ssRNA comprises 25 or fewer nucleotides, non-covalently bound to a dye of the Formula I:

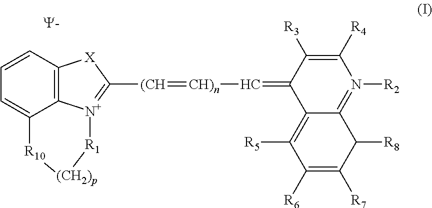

w wherein
X is O or S;
n is 0, 1 or 2;
p is 0, 1 or 2;
Ψ is a biologically compatible counterion;
R₁ and R₁₀, which can be the same or different, are independently H; or a substituted or unsubstituted alkyl group, wherein the substitution on the alkyl group, if present, is a carboxyl or (alkyl)ammonium group;
R₂ is a substituted or unsubstituted alkyl, naphthyl, phenyl, thienyl, or cycloalkyl having 3-8 carbons, wherein the substitution on R₂, if present, is a carboxyl or carboxamide group;
R₃ is H; or an alkyl, alkenyl, polyalkenyl, alkynyl or polyalkynyl group; or a halogen; or a substituted or unsubstituted aryl or heteroaryl; or a substituted or unsubstituted cycloalkyl having 3-10 carbons; or —OR₁₁, —SR₁₁, —(NR₉R₁₁); or —OSO₂R₁₉; or a TAIL;
wherein R₉ and R₁₁, which can be the same or different, are independently H; or substituted or unsubstituted alkyl groups; or alkyl groups substituted with one or more alkylammonium groups; or 1-2 alicyclic or aromatic rings; or R₉ and R₁₁ taken in combination are —(CH₂)₄— or —(CH₂)₅— to give a 5 or 6 membered ring; and where R₁₉ is alkyl, or perfluoroalkyl, or aryl;
R₄ is an alkyl, alkenyl, polyalkenyl, alkynyl or polyalkynyl group; or a halogen; or a substituted or unsubstituted aryl or heteroaryl; or a substituted or unsubstituted cycloalkyl having 3-10 carbons; or —OR₁₁, —SR₁₁, —(NR₉R₁₁); or —OSO₂R₁₉; or a TAIL;
R₅, R₆, R₇ and R₈, which can be the same or different, are independently H; or an alkyl, alkenyl, polyalkenyl, alkynyl or polyalkynyl group; or a halogen; or a substituted or unsubstituted aryl or heteroaryl; or a substituted or unsubstituted cycloalkyl having 3-8 carbons; or a TAIL; or —OR₁₁, —SR₁₁, or —(NR₉R₁₁);
TAIL is a heteroatom-containing moiety having the formula LINK-SPACER-CAP;
wherein
LINK is a single covalent bond, —O—, —S—, or —NR₂₀; where R₂₀ is H, a linear or branched alkyl having 1-8 carbons, or R₂₀ is —SPACER′-CAP′;
SPACER and SPACER′, which can be the same or different, are covalent linkages, linear or branched, cyclic or heterocyclic, saturated or unsaturated, each having 1-16 nonhydrogen atoms selected from the group consisting of C, N, O and S, such that the linkage begins and ends with a carbon atom, and contains any combination of ether, thioether, amine, ester, amide, or aliphatic, oleic or aromatic carbon-carbon bonds, or aromatic carbon-nitrogen or nitrogen-nitrogen bonds; wherein all heteroatoms in the linear backbone of SPACER are separated by at least two carbon atoms;
CAP and CAP′, which can be the same or different, are —OR₂₁, —SR₂₁, —NR₂₁R₂₂, or —N⁺R₂₁R₂₂R₂₃Ψ⁻;
wherein
R₂₁, R₂₂, and R₂₃ are independently H, or a linear or branched alkyl or cycloalkyl having 1-8 carbons, optionally further substituted by halogen, hydroxy, alkoxy having 1-8 carbons, carboxyalkyl having 1-8 carbons, or phenyl, where phenyl is optionally further substituted by halogen, hydroxy, alkoxy having 1-8 carbons, aminoalkyl having 1-8 carbons, or carboxyalkyl having 1-8 carbons; or, one or more of R₂₁, R₂₂, and R₂₃, taken in combination with SPACER or SPACER′ or R₂₀ forms a 5- or 6-membered aromatic, heteroaromatic, alicyclic or heteroalicyclic ring, the heteroatoms selected from O, N or S; where Ψ⁻ is a biologically compatible counterion; or
CAP and CAP′ are independently

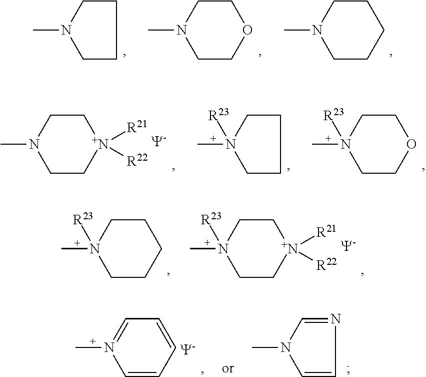

where R₂₁, R₂₂, R₂₃ and Ψ⁻ are as defined previously; such that at least one of R₃, R₄, R₅, R₆, R₇, R₈ or R₁₀ is a TAIL; and, where more than one substituent is a TAIL, each TAIL is optionally the same or different; or
or wherein at least one of R₁, R₅, R₆, R₇, R₈, R₉, R₁₀, R₁₁ or R₂₀, R₂₁, R₂₂ or R₂₃ is a water-soluble group (WSG);

wherein the complex emits a detectable fluorescent signal.

When CAP is —N⁺R₂₁R₂₂R₂₃Ψ⁻, the biologically compatible counter ion balances the positive charge present on the CAP nitrogen, which is a quaternary ammonium salt. As used herein, a substance that is biologically compatible is not toxic as used, and does not have a substantially deleterious effect on biomolecules. Examples of Ψ⁻ include, among others, chloride, bromide, iodide, sulfate, alkanesulfonate, arylsulfonate, phosphate, perchlorate, tetrafluoroboronate, tetraarylboride, nitrate, and anions of aromatic or aliphatic carboxylic acids. Preferred ψ-counterions are bromide, chloride, iodide, perchlorate, and various sulfonates.

The water-soluble group can include a polymer (e.g., a polyalkylene oxide, carbohydrate and polypeptide). In certain embodiments, the water-soluble group comprises a polyethylene oxide. In certain embodiments, R₄ is a TAIL; R₂ is a substituted or unsubstituted phenyl; and/or at least one of R₅, R₆, R₇ and R₈ is —OR₁₁, —SR₁₁, or —NR₉R₁₁). In certain embodiments, n is 0. In certain embodiments, at least one of R₅, R₆, R₇ and R₈ is —OR₁₁. In certain embodiments, OR₁₁ is —OCH₃. In certain embodiments, R₇ is —OCH₃. In certain embodiments, R₁ is an unsubstituted alkyl and R₁₀ is H. In certain embodiments, R₁ is methyl and R₁₀ is H.

In certain embodiments, wherein if p is 0, and wherein R₁ and R₁₀, which can be the same or different, are independently H; or a substituted or unsubstituted alkyl group, wherein the substitution on the alkyl group, if present, is a carboxyl or (alkyl)ammonium group. In other embodiments, wherein if p is not 0, R₁ and R₁₀, which can be the same or different, are independently a substituted or unsubstituted alkyl group, wherein the substitution on the alkyl group, if present, is a carboxyl or (alkyl)ammonium group.

In certain embodiments, the dye compound of Formula I is selected from the group consisting of Compound 4, Compound 5, Compound 6, Compound 7, Compound 8, Compound 9, Compound 10, Compound 11, Compound 12, Compound 13, Compound 14, Compound 15, Compound 16, Compound 17, Compound 18, Compound 19, Compound 20, Compound 21, Compound 23, Compound 25, Compound 26, Compound 27, Compound 30, Compound 31, Compound 32, Compound 33, Compound 34, Compound 35, Compound 36, Compound 37, Compound 38, Compound 39, Compound 40, Compound 41, Compound 42, Compound 43, Compound 44, Compound 45, Compound 46, Compound 47, Compound 48, Compound 49, Compound 50, Compound 51, Compound 52, Compound 53, Compound 54, Compound 55, Compound 56, Compound 57, Compound 58, Compound 59, Compound 60, Compound 61, Compound 62, Compound 63, Compound 64, and Compound 65.

In certain embodiments, the short RNA molecule can be a single-stranded RNA (ssRNA) molecule. The ssRNA can be naturally occurring or can include a chemical modification to at least one sugar, phosphate or base group. For example, the ssRNA can include a chemical group other than a hydroxy or phosphate at its 5′ terminus. In certain embodiments, the ssRNA molecule can include 20-25 nucleotides. In other embodiments, the ssRNA molecule can include 10-20 nucleotides. In certain methods, the ssRNA is microRNA (miRNA). The ssRNA can be a small nuclear RNA (snRNA), small nucleolar RNA (snoRNA), tiny non-coding RNA (tncRNA), small modulatory RNA (smRNA) or piwi-interacting RNA (piRNA).

In yet another aspect, a fluorescent complex is produced that includes ssRNA, wherein the ssRNA comprises 25 or fewer nucleotides, non-covalently bound to a dye compound selected from the group consisting of Compound 4, Compound 5, Compound 6, Compound 7, Compound 8, Compound 9, Compound 10, Compound 11, Compound 12, Compound 13, Compound 14, Compound 15, Compound 16, Compound 17, Compound 18, Compound 19, Compound 20, Compound 21, Compound 23, Compound 25, Compound 26, Compound 27, Compound 30, Compound 31, Compound 32, Compound 33, Compound 34, Compound 35, Compound 36, Compound 37, Compound 38, Compound 39, Compound 40, Compound 41, Compound 42, Compound 43, Compound 44, Compound 45, Compound 46, Compound 47, Compound 48, Compound 49, Compound 50, Compound 51, Compound 52, Compound 53, Compound 54, Compound 55, Compound 56, Compound 57, Compound 58, Compound 59, Compound 60, Compound 61, Compound 62, Compound 63, Compound 64, and Compound 65, wherein the complex emits a detectable signal.

In certain embodiments, the short RNA molecule can be a single-stranded RNA (ssRNA) molecule. The ssRNA can be naturally occurring or can include a chemical modification to at least one sugar, phosphate or base group. For example, the ssRNA can include a chemical group other than a hydroxy or phosphate at its 5′ terminus. In certain embodiments, the ssRNA molecule can include 20-25 nucleotides. In other embodiments, the ssRNA molecule can include 10-20 nucleotides. In certain methods, the ssRNA is microRNA (miRNA). The ssRNA can be a small nuclear RNA (snRNA), small nucleolar RNA (snoRNA), tiny non-coding RNA (tncRNA), small modulatory RNA (smRNA) or piwi-interacting RNA (piRNA).

In yet another aspect, a fluorescent complex is produced that includes ssRNA, wherein the ssRNA comprises 25 or fewer nucleotides, non-covalently bound to a substituted, unsymmetrical cyanine dye compound, wherein the dye comprises a first heterocyclic ring system that is a substituted benzothiazolium, benzoxazolium, benzoselenazolium, benzimidazolium, or dialkylindolinium, wherein the first ring system is linked by a monomethine or trimethine bridging moiety attached at the 2-position of the first ring system to the 2- or 4-position of a second heterocyclic ring system that is a substituted quinolinium or that is linked by a monomethine bridging moiety attached to the 2-position of a second heterocyclic ring system that is a substituted pyridinium; wherein one or more substituents of the second ring system is a substituted or unsubstituted cyclohexyl, cyclohexenyl, morpholino, piperidinyl, naphthyl, phenyl, thienyl, benzothizolyl, furanyl, oxazolyl, benzoxazolyl or pyridinyl group, wherein the complex emits a detectable signal.

In certain embodiments, the short RNA molecule can be a single-stranded RNA (ssRNA) molecule. The ssRNA can be naturally occurring or can include a chemical modification to at least one sugar, phosphate or base group. For example, the ssRNA can include a chemical group other than a hydroxy or phosphate at its 5′ terminus. In certain embodiments, the ssRNA molecule can include 20-25 nucleotides. In other embodiments, the ssRNA molecule can include 10-20 nucleotides. In certain methods, the ssRNA is microRNA (miRNA). The ssRNA can be a small nuclear RNA (snRNA), small nucleolar RNA (snoRNA), tiny non-coding RNA (tncRNA), small modulatory RNA (smRNA) or piwi-interacting RNA (piRNA).

The fluorescent signal generated by the fluorescent complex is extremely stable. For example, the signal can be stable for at least 30 minutes at room temperature. In some embodiments, the fluorescence signal is stable for at least 3 hours at room temperature.

In yet another aspect, a kit for detecting the presence of single-stranded RNA (ssRNA) in a sample is provided that includes:

a dye of the Formula II; and

instructions for using the dye in a method as disclosed herein.

In yet another aspect, a kit for detecting the presence of single-stranded RNA (ssRNA) in a sample is provided that includes:

a dye of the Formula I:

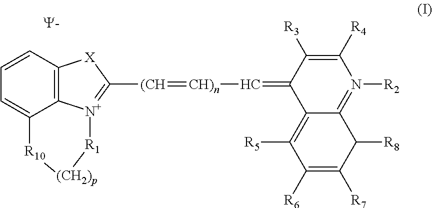

wherein
X is O or S;
n is 0, 1 or 2;
p is 0, 1 or 2;
Ψ is a biologically compatible counterion;
R₁ and R₁₀, which can be the same or different, are independently H; or a substituted or unsubstituted alkyl group, wherein the substitution on the alkyl group, if present, is a carboxyl or (alkyl)ammonium group;
R₂ is a substituted or unsubstituted alkyl, naphthyl, phenyl, thienyl, or cycloalkyl having 3-8 carbons, wherein the substitution on R₂, if present, is a carboxyl or carboxamide group;
R₃ is H; or an alkyl, alkenyl, polyalkenyl, alkynyl or polyalkynyl group; or a halogen; or a substituted or unsubstituted aryl or heteroaryl; or a substituted or unsubstituted cycloalkyl having 3-10 carbons; or —OR₁₁, —SR₁₁, —(NR₉R₁₁); or —OSO₂R₁₉;
wherein R₉ and R₁₁, which can be the same or different, are independently H; or substituted or unsubstituted alkyl groups; or alkyl groups substituted with one or more alkylammonium groups; or 1-2 alicyclic or aromatic rings; or R₉ and R₁₁ taken in combination are —(CH₂)₄— or —(CH₂)₅— to give a 5 or 6 membered ring; and where R₁₉ is alkyl, or perfluoroalkyl, or aryl;
R₄ is an alkyl, alkenyl, polyalkenyl, alkynyl or polyalkynyl group; or a halogen; or a substituted or unsubstituted aryl or heteroaryl; or a substituted or unsubstituted cycloalkyl having 3-10 carbons; or —OR₁₁, —SR₁₁, —(NR₉R₁₁); or —OSO₂R₁₉; or a TAIL;
R₅, R₆, R₇ and R₈, which can be the same or different, are independently H; or an alkyl, alkenyl, polyalkenyl, alkynyl or polyalkynyl group; or a halogen; or a substituted or unsubstituted aryl or heteroaryl; or a substituted or unsubstituted cycloalkyl having 3-8 carbons; or a TAIL; or —OR₁₁, —SR₁₁, or —(NR₉R₁₁);
TAIL is a heteroatom-containing moiety having the formula LINK-SPACER-CAP;
wherein
LINK is a single covalent bond, —O—, —S—, or —NR₂₀; where R₂₀ is H, a linear or branched alkyl having 1-8 carbons, or R₂₀ is SPACER′-CAP′;
SPACER and SPACER′, which can be the same or different, are covalent linkages, linear or branched, cyclic or heterocyclic, saturated or unsaturated, each having 1-16 nonhydrogen atoms selected from the group consisting of C, N, O and S, such that the linkage begins and ends with a carbon atom, and contains any combination of ether, thioether, amine, ester, amide, or aliphatic, oleic or aromatic carbon-carbon bonds, or aromatic carbon-nitrogen or nitrogen-nitrogen bonds; wherein all heteroatoms in the linear backbone of SPACER are separated by at least two carbon atoms;
CAP and CAP′, which can be the same or different, are —OR₂₁, —SR₂₁, —NR₂₁R₂₂, or —N⁺R₂₁R₂₂R₂₃Ψ⁻;
wherein
R₂₁, R₂₂, and R₂₃ are independently H, or a linear or branched alkyl or cycloalkyl having 1-8 carbons, optionally further substituted by halogen, hydroxy, alkoxy having 1-8 carbons, carboxyalkyl having 1-8 carbons, or phenyl, where phenyl is optionally further substituted by halogen, hydroxy, alkoxy having 1-8 carbons, aminoalkyl having 1-8 carbons, or carboxyalkyl having 1-8 carbons; or, one or more of R₂₁, R₂₂, and R₂₃, taken in combination with SPACER or SPACER′ or R₂₀ forms a 5- or 6-membered aromatic, heteroaromatic, alicyclic or heteroalicyclic ring, the heteroatoms selected from O, N or S; where Ψ⁻ is a biologically compatible counterion; or
CAP and CAP′ are independently

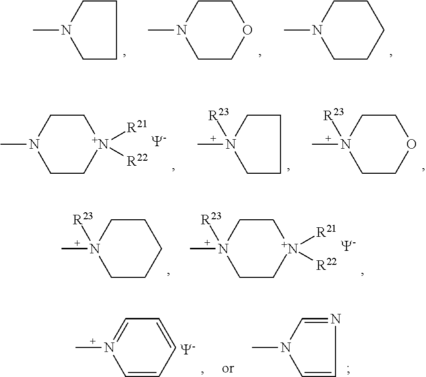

where R₂₁, R₂₂, R₂₃ and Ψ⁻ are as defined previously; such that at least one of R₃, R₄, R₅, R₆, R₇, R₈ or R₁₀ is a TAIL; and, where more than one substituent is a TAIL, each TAIL is optionally the same or different; or
or wherein at least one of R₁, R₅, R₆, R₇, R₈, R₉, R₁₀, R₁₁ or R₂₀, R₂₁, R₂₂ or R₂₃ is a water-soluble group (WSG); and

instructions for using the dye in a method as disclosed herein.

When CAP is —N⁺R₂₁R₂₂R₂₃Ψ⁻, the biologically compatible counter ion balances the positive charge present on the CAP nitrogen, which is a quaternary ammonium salt. As used herein, a substance that is biologically compatible is not toxic as used, and does not have a substantially deleterious effect on biomolecules. Examples of Ψ⁻ include, among others, chloride, bromide, iodide, sulfate, alkanesulfonate, arylsulfonate, phosphate, perchlorate, tetrafluoroboronate, tetraarylboride, nitrate, and anions of aromatic or aliphatic carboxylic acids. Preferred ψ-counterions are bromide, chloride, iodide, perchlorate, and various sulfonates.

The water-soluble group can include a polymer (e.g., a polyalkylene oxide, carbohydrate and polypeptide). In certain embodiments, the water-soluble group comprises a polyethylene oxide. In certain embodiments, R₄ is a TAIL; R₂ is a substituted or unsubstituted phenyl; and/or at least one of R₅, R₆, R₇ and R₈ is —OR₁₁, —SR₁₁, or —NR₉R₁₁). In certain embodiments, n is 0. In certain embodiments, at least one of R₅, R₆, R₇ and R₈ is —OR₁₁. In certain embodiments, OR₁₁ is —OCH₃. In certain embodiments, R₇ is —OCH₃. In certain embodiments, R₁ is an unsubstituted alkyl and R₁₀ is H. In certain embodiments, R₁ is methyl and R₁₀ is H.

In certain embodiments, wherein if p is 0, and wherein R₁ and R₁₀, which can be the same or different, are independently H; or a substituted or unsubstituted alkyl group, wherein the substitution on the alkyl group, if present, is a carboxyl or (alkyl)ammonium group. In other embodiments, wherein if p is not 0, R₁ and R₁₀, which can be the same or different, are independently a substituted or unsubstituted alkyl group, wherein the substitution on the alkyl group, if present, is a carboxyl or (alkyl)ammonium group.

In certain embodiments, the dye compound of Formula I is selected from the group consisting of Compound 4, Compound 5, Compound 6, Compound 7, Compound 8, Compound 9, Compound 10, Compound 11, Compound 12, Compound 13, Compound 14, Compound 15, Compound 16, Compound 17, Compound 18, Compound 19, Compound 20, Compound 21, Compound 23, Compound 25, Compound 26, Compound 27, Compound 30, Compound 31, Compound 32, Compound 33, Compound 34, Compound 35, Compound 36, Compound 37, Compound 38, Compound 39, Compound 40, Compound 41, Compound 42, Compound 43, Compound 44, Compound 45, Compound 46, Compound 47, Compound 48, Compound 49, Compound 50, Compound 51, Compound 52, Compound 53, Compound 54, Compound 55, Compound 56, Compound 57, Compound 58, Compound 59, Compound 60, Compound 61, Compound 62, Compound 63, Compound 64, and Compound 65.

In yet another aspect, a kit for detecting the presence of single-stranded RNA (ssRNA) in a sample is provided that includes:

a dye compound selected from the group consisting of Compound 4, Compound 5, Compound 6, Compound 7, Compound 8, Compound 9, Compound 10, Compound 11, Compound 12, Compound 13, Compound 14, Compound 15, Compound 16, Compound 17, Compound 18, Compound 19, Compound 20, Compound 21, Compound 23, Compound 25, Compound 26, Compound 27, Compound 30, Compound 31, Compound 32, Compound 33, Compound 34, Compound 35, Compound 36, Compound 37, Compound 38, Compound 39, Compound 40, Compound 41, Compound 42, Compound 43, Compound 44, Compound 45, Compound 46, Compound 47, Compound 48, Compound 49, Compound 50, Compound 51, Compound 52, Compound 53, Compound 54, Compound 55, Compound 56, Compound 57, Compound 58, Compound 59, Compound 60, Compound 61, Compound 62, Compound 63, Compound 64, and Compound 65; and

instructions for using the dye in a method as disclosed herein.

In yet another aspect, a kit for detecting the presence of single-stranded RNA (ssRNA) in a sample is provided that includes:

a substituted, unsymmetrical cyanine dye compound, wherein the dye comprises a first heterocyclic ring system that is a substituted benzothiazolium, benzoxazolium, benzoselenazolium, benzimidazolium, or dialkylindolinium, wherein the first ring system is linked by a monomethine or trimethine bridging moiety attached at the 2-position of the first ring system to the 2- or 4-position of a second heterocyclic ring system that is a substituted quinolinium or that is linked by a monomethine bridging moiety attached to the 2-position of a second heterocyclic ring system that is a substituted pyridinium; wherein one or more substituents of the second ring system is a substituted or unsubstituted cyclohexyl, cyclohexenyl, morpholino, piperidinyl, naphthyl, phenyl, thienyl, benzothizolyl, furanyl, oxazolyl, benzoxazolyl or pyridinyl group; and

instructions for using the dye in a method as disclosed herein.

In yet another aspect, dye compounds are provided, the dye compounds comprising the structure of Formula I:

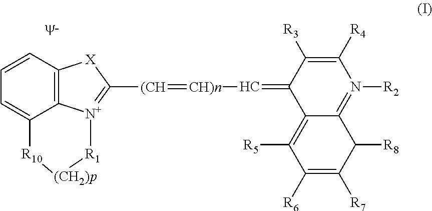

wherein
X is O or S;
n is 0, 1 or 2;
p is 0, 1 or 2;
Ψ is a biologically compatible counterion;
R₁ and R₁₀, which can be the same or different, are independently H; or a substituted or unsubstituted alkyl group, wherein the substitution on the alkyl group, if present, is a carboxyl or (alkyl)ammonium group;
R₂ is a substituted or unsubstituted alkyl, naphthyl, phenyl, thienyl, or cycloalkyl having 3-8 carbons, wherein the substitution on R₂, if present, is a carboxyl or carboxamide group;
R₃ is H; or an alkyl, alkenyl, polyalkenyl, alkynyl or polyalkynyl group; or a halogen; or a substituted or unsubstituted aryl or heteroaryl; or a substituted or unsubstituted cycloalkyl having 3-10 carbons; or —OR₁₁, —SR₁₁, —(NR₉R₁₁); or —OSO₂R₁₉; or a TAIL;
wherein R₉ and R₁₁, which can be the same or different, are independently H; or substituted or unsubstituted alkyl groups; or alkyl groups substituted with one or more alkylammonium groups; or 1-2 alicyclic or aromatic rings; or R₉ and R₁₁ taken in combination are —(CH₂)₄— or —(CH₂)₅— to give a 5 or 6 membered ring; and where R₁₉ is alkyl, or perfluoroalkyl, or aryl;
R₄ is an alkyl, alkenyl, polyalkenyl, alkynyl or polyalkynyl group; or a halogen; or a substituted or unsubstituted aryl or heteroaryl; or a substituted or unsubstituted cycloalkyl having 3-10 carbons; or —OR₁₁, —SR₁₁, —(NR₉R₁₁); or —OSO₂R₁₉; or a TAIL;
R₅, R₆, R₇ and R₈, which can be the same or different, are independently H; or an alkyl, alkenyl, polyalkenyl, alkynyl or polyalkynyl group; or a halogen; or a substituted or unsubstituted aryl or heteroaryl; or a substituted or unsubstituted cycloalkyl having 3-8 carbons; or a TAIL; or —OR₁₁, —SR₁₁, or —(NR₉R₁₁;
TAIL is a heteroatom-containing moiety having the formula LINK-SPACER-CAP;
wherein
LINK is a single covalent bond, —O—, —S—, or —NR₂₀; where R₂₀ is H, a linear or branched alkyl having 1-8 carbons, or R₂₀ is -SPACER′-CAP′;
SPACER and SPACER′, which can be the same or different, are covalent linkages, linear or branched, cyclic or heterocyclic, saturated or unsaturated, each having 1-16 nonhydrogen atoms selected from the group consisting of C, N, O and S, such that the linkage begins and ends with a carbon atom, and contains any combination of ether, thioether, amine, ester, amide, or aliphatic, oleic or aromatic carbon-carbon bonds, or aromatic carbon-nitrogen or nitrogen-nitrogen bonds; wherein all heteroatoms in the linear backbone of SPACER are separated by at least two carbon atoms;
CAP and CAP′, which can be the same or different, are —OR₂₁, —SR₂₁, —NR₂₁R₂₂, or —N⁺R₂₁R₂₂R₂₃Ψ⁻;
wherein
R₂₁, R₂₂, and R₂₃ are independently H, or a linear or branched alkyl or cycloalkyl having 1-8 carbons, optionally further substituted by halogen, hydroxy, alkoxy having 1-8 carbons, carboxyalkyl having 1-8 carbons, or phenyl, where phenyl is optionally further substituted by halogen, hydroxy, alkoxy having 1-8 carbons, aminoalkyl having 1-8 carbons, or carboxyalkyl having 1-8 carbons; or, one or more of R₂₁, R₂₂, and R₂₃, taken in combination with SPACER or SPACER′ or R₂₀ forms a 5- or 6-membered aromatic, heteroaromatic, alicyclic or heteroalicyclic ring, the heteroatoms selected from O, N or S; where Ψ⁻ is a biologically compatible counterion; or
CAP and CAP′ are independently

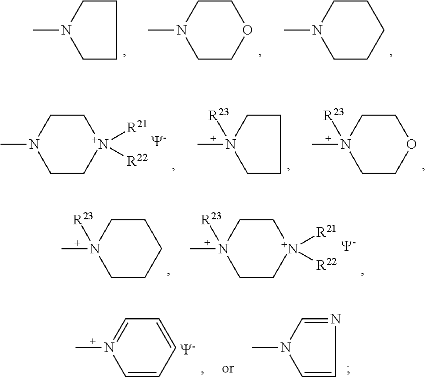

where R₂₁, R₂₂, R₂₃ and Ψ⁻ are as defined previously; such that at least one of R₃, R₄, R₅, R₆, R₇, R₈ or R₁₀ is a TAIL; and, where more than one substituent is a TAIL, each TAIL is optionally the same or different; or
or wherein at least one of R₁, R₅, R₆, R₇, R₈, R₉, R₁₀, R₁₁ or R₂₀, R₂₁, R₂₂ or R₂₃ is a water-soluble group (WSG).

When CAP is —N⁺R₂₁R₂₂R₂₃Ψ⁻, the biologically compatible counter ion balances the positive charge present on the CAP nitrogen, which is a quaternary ammonium salt. As used herein, a substance that is biologically compatible is not toxic as used, and does not have a substantially deleterious effect on biomolecules. Examples of Ψ⁻ include, among others, chloride, bromide, iodide, sulfate, alkanesulfonate, arylsulfonate, phosphate, perchlorate, tetrafluoroboronate, tetraarylboride, nitrate, and anions of aromatic or aliphatic carboxylic acids. Preferred ψ-counterions are bromide, chloride, iodide, perchlorate, and various sulfonates.

The water-soluble group can include a polymer (e.g., a polyalkylene oxide, carbohydrate and polypeptide). In certain embodiments, the water-soluble group comprises a polyethylene oxide. In certain embodiments, R₄ is a TAIL; R₂ is a substituted or unsubstituted phenyl; and/or at least one of R₅, R₆, R₇ and R₈ is —OR₁₁, —SR₁₁, or —NR₁₁R₉). In certain embodiments, n is 0. In certain embodiments, at least one of R₅, R₆, R₇ and R₈ is —OR₁₁. In certain embodiments, OR₁₁ is —OCH₃. In certain embodiments, R₇ is —OCH₃. In certain embodiments, R₁ is an unsubstituted alkyl and R₁₀ is H. In certain embodiments, R₁ is methyl and R₁₀ is H.

In certain embodiments, wherein if p is 0, and wherein R₁ and R₁₀, which can be the same or different, are independently H; or a substituted or unsubstituted alkyl group, wherein the substitution on the alkyl group, if present, is a carboxyl or (alkyl)ammonium group. In other embodiments, wherein if p is not 0, R₁ and R₁₀, which can be the same or different, are independently a substituted or unsubstituted alkyl group, wherein the substitution on the alkyl group, if present, is a carboxyl or (alkyl)ammonium group.

In certain embodiments, the dye compound of Formula I is selected from the group consisting of Compound 4, Compound 5, Compound 6, Compound 7, Compound 8, Compound 9, Compound 10, Compound 11, Compound 12, Compound 13, Compound 14, Compound 15, Compound 16, Compound 17, Compound 18, Compound 19, Compound 20, Compound 21, Compound 23, Compound 25, Compound 26, Compound 27, Compound 30, Compound 31, Compound 32, Compound 33, Compound 34, Compound 35, Compound 36, Compound 37, Compound 38, Compound 39, Compound 40, Compound 41, Compound 42, Compound 43, Compound 44, Compound 45, Compound 46, Compound 47, Compound 48, Compound 49, Compound 50, Compound 51, Compound 52, Compound 53, Compound 54, Compound 55, Compound 56, Compound 57, Compound 58, Compound 59, Compound 60, Compound 61, Compound 62, Compound 63, Compound 64, and Compound 65.

These and other features, aspects, and embodiments are described herein.

### BRIEF DESCRIPTION OF THE DRAWINGS

The skilled artisan will understand that the drawings described below are for illustration purposes only. The drawings are not intended to limit the scope of the present teachings in any way.

For a more complete understanding of the principles disclosed herein, and the advantages thereof, reference is now made to the following descriptions taken in conjunction with the accompanying drawings, in which:

FIG. 1A is a plot showing the line corresponding to the curve-fitting algorithm (a Modified Hill plot) used to calculate concentration in the MyQubit miRNA Assay. For reference, the positions of the standards and a set of data points from an actual experiment are shown superimposed onto the line, demonstrating that the curve-fitting algorithm gives accurate values for quantitation. FIG. 1B is a computer screen shot of the QUBIT assay.

FIG. 2 is a screen shot of the upload of the MyQubit miRNA Assay.

FIG. 3 is a graphic representation of RNA assays according to certain embodiments of the methods disclosed herein wherein the selectivity for siRNA (small double stranded RNA) over rRNA (ribosomal RNA).

FIG. 4 is a graphic representation of RNA assays according to certain embodiments of the methods disclosed herein wherein the assay detects small ssRNA and dsRNA.

FIG. 5 is a graphic representation of RNA assays according to certain embodiments of the methods disclosed herein wherein the miRNA assay detects only a portion of the total RNA in the sample (extracted from U2OS cells).

FIGS. 6A-6D are graphic representations of RNA assays according to certain embodiments of the methods disclosed herein wherein the MyQubit miRNA Assay detected about 15% of each mRNA (FIGS. 6A and 6B), and the QUBIT RNA Assay detected 90-150% of each mRNA (FIGS. 6C and 6D).

FIGS. 7A-7H are graphic representations of RNA assays according to certain embodiments of the methods disclosed herein wherein various dye compounds of the present teachings are used.

### DETAILED DESCRIPTION

The embodiments described herein may be understood more readily by reference to the following detailed description of the embodiments and the Examples included herein. It is to be understood that the terminology used herein is for the purpose of describing specific embodiments only and is not intended to be limiting.

The entirety of each patent, patent application, publication and document referenced in this disclosure is hereby incorporated by reference in its entirety, including all tables, drawings, and figures.

### DEFINITIONS

Unless defined otherwise, all technical and scientific terms used herein have the same meaning as is commonly understood by one of ordinary skill in the art.

As used herein, “a” or “an” means “at least one” or “one or more”.

As used herein, “about” means that the numerical value is approximate and small variations would not significantly affect the use and practice of the compositions and methods provided herein. Where a numerical limitation is used, unless indicated otherwise by the context, “about” means the numerical value can vary by ±10% and remain within the scope of the disclosed embodiments.

The term “or combinations thereof” as used herein refers to all permutations and combinations of the listed terms preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Certain compounds of the present disclosure can exist in unsolvated forms as well as solvated forms, including hydrated forms. In general, the solvated forms are equivalent to unsolvated forms and are encompassed within the scope of the present disclosure. Certain compounds disclosed herein may exist in multiple crystalline or amorphous forms. In general, all physical forms are equivalent for the uses contemplated by the present disclosure and are intended to be within the scope of the present teachings.

Certain compounds disclosed herein possess asymmetric carbon atoms (optical centers) or double bonds; the racemates, diastereomers, geometric isomers and individual isomers are encompassed within the scope of the present disclosure.

The compounds described herein may be prepared as a single isomer (e.g., enantiomer, cis-trans, positional, diastereomer) or as a mixture of isomers. In a preferred embodiment, the compounds are prepared as substantially a single isomer. Methods of preparing substantially isomerically pure compounds are known in the art. For example, enantiomerically enriched mixtures and pure enantiomeric compounds can be prepared by using synthetic intermediates that are enantiomerically pure in combination with reactions that either leave the stereochemistry at a chiral center unchanged or result in its complete inversion. Alternatively, the final product or intermediates along the synthetic route can be resolved into a single stereoisomer. Techniques for inverting or leaving unchanged a particular stereocenter, and those for resolving mixtures of stereoisomers are well known in the art and it is well within the ability of one of skill in the art to choose and appropriate method for a particular situation. See, generally, Furniss et al. (eds.), VOGEL'S ENCYCLOPEDIA OF PRACTICAL ORGANIC CHEMISTRY 5^{TH} ED., Longman Scientific and Technical Ltd., Essex, 1991, pp. 809-816; and Heller, Acc. Chem. Res. 23: 128 (1990).

The compounds disclosed herein may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds disclosed herein, whether radioactive or not, are intended to be encompassed within the scope of the present disclosure.

Where a disclosed compound includes a conjugated ring system, resonance stabilization may permit a formal electronic charge to be distributed over the entire molecule. While a particular charge may be depicted as localized on a particular ring system, or a particular heteroatom, it is commonly understood that a comparable resonance structure can be drawn in which the charge may be formally localized on an alternative portion of the compound.

Selected compounds having a formal electronic charge may be shown without an appropriate biologically compatible counterion. Such a counterion serves to balance the positive or negative charge present on the compound. As used herein, a substance that is biologically compatible is not toxic as used, and does not have a substantially deleterious effect on biomolecules. Examples of negatively charged counterions include, among others, chloride, bromide, iodide, sulfate, alkanesulfonate, arylsulfonate, phosphate, perchlorate, tetrafluoroborate, tetraarylboride, nitrate and anions of aromatic or aliphatic carboxylic acids. Preferred counterions may include chloride, iodide, perchlorate and various sulfonates. Examples of positively charged counterions include, among others, alkali metal, or alkaline earth metal ions, ammonium, or alkylammonium ions.

Where substituent groups are specified by their conventional chemical formulae, written from left to right, they equally encompass the chemically identical substituents, which would result from writing the structure from right to left, e.g., —CH₂O— is intended to also recite —OCH₂—.

A dashed line projecting from a substituent, such as:

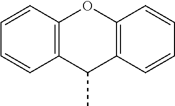

indicates the point of attachment to the base molecule. For a fused ring, dashed lines indicate portions of the base molecule where the fused ring is attached, such as:

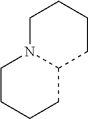

wherein the full molecule could have the structure:

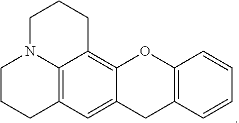 [SMILES: c1ccc2c(c1)Cc1cc3c4c(c1O2)CCCN4CCC3]

The term “alkyl,” by itself or as part of another substituent means, unless otherwise stated, a straight or branched chain, or cyclic hydrocarbon radical, or combination thereof, which may be fully saturated, mono- or polyunsaturated and can include divalent (“alkylene”) and multivalent radicals, having the number of carbon atoms designated (e.g. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups that are limited to hydrocarbon groups are termed “homoalkyl”.

Exemplary alkyl groups of use in the present disclosure contain between about one and about twenty five carbon atoms (e.g. methyl, ethyl and the like). Straight, branched or cyclic hydrocarbon chains having eight or fewer carbon atoms will also be referred to herein as “lower alkyl”. In addition, the term “alkyl” as used herein further includes one or more substitutions at one or more carbon atoms of the hydrocarbon chain fragment.

As used herein, “aryl” refers to an aromatic moiety having a single ring or multiple condensed rings each of which is optionally and independently substituted with H, halogen, cyano, azido, sulfonic acid, alkali or ammonium salt of sulfonic acid, carboxylic acid, biologically compatible salt of carboxylic acid, nitro, alkyl, perfluoroalkyl, alkoxy, alkylthio, amino, monoalkylamino, dialkylamino or alkylamido.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a straight or branched chain, or cyclic carbon-containing radical, or combinations thereof, consisting of the stated number of carbon atoms and at least one heteroatom selected from the group consisting of O, N, Si, P, S, and Se and wherein the nitrogen, phosphorous, sulfur, and selenium atoms are optionally oxidized, and the nitrogen heteroatom is optionally be quaternized. The heteroatom(s) O, N, P, S, Si, and Se may be placed at any interior position of the heteroalkyl group or at the position at which the alkyl group is attached to the remainder of the molecule. Examples include, but are not limited to, —CH₂—CH₂—O—CH₃, —CH₂—CH₂NH—CH₃, —CH₂—CH₂N(CH₃)—CH₃, —CH₂S—CH₂—CH₃, —CH₂—CH₂, —S(O)CH₃, —CH₂—CH₂S(O)₂—CH₃, —CH═CH—O—CH₃, —Si(CH₃)₃, —CH₂CH═N—OCH₃, and —CH═CH—N(CH₃)—CH₃. Up to two heteroatoms may be consecutive, such as, for example, —CH₂NH—OCH₃ and CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂S—CH₂—CH₂NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and R′C(O)₂—.

As used herein, “heteroaryl” refers to a 5- or 6-membered aromatic heterocycle that is optionally fused to an additional six-membered aromatic ring or to one 5- or 6-membered heteroaromatic ring, said heteroaromatic ring containing 1-3 heteroatoms that are selected from the group consisting of O, N and S in any combination. Any heteroaryl substituent is attached by a single bond, and is optionally and independently substituted one or more times with H, halogen, alkyl having 1-6 carbons, or alkoxy having 1-6 carbons. Selected examples of heteroaryl substituents are pyrrole, thiophene, or furan (single ring, single hetero atom), oxazole, isoxazole, oxadiazole, or imidazole (single ring, multiple hetero atoms). Examples of multi-ring heteroaryl groups include benzoxazole, benzothiazole, benzimidazole (multi-ring, multiple hetero atoms), benzofuran or indole (multi-ring, single hetero atom).

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

As used herein, “alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

As used herein, “alkenyl” refers to alkenyl groups having from 2 to 22 carbon atoms, preferably 2 to 4 carbon atoms, and having at least 1 and preferably from 1 to 2 sites of alkenyl unsaturation. Such groups are exemplified, for example, by vinyl, allyl, and but-3-en-1-yl.

As used herein, “alkenoxy” refers to the group —O-alkenyl wherein alkenyl is defined herein. Alkenoxy includes, by way of example, vinyloxy, allyloxy, 1-butenoxy, 2-butenoxy, 2-pentenoxy, 3-pentenoxy, 4-pentenoxy.

As used herein, “alkylaryl” refers to an alkyl group substituted with an aryl group (e.g., an aromatic group or an heteroaromatic group).

As used herein, “heterocycle” or “heterocyclic” or “heterocycloalkyl” or “heterocyclyl” refers to a saturated or unsaturated group having a single ring or multiple condensed rings, including fused bridged and spiro ring systems, from 1 to 10 carbon atoms and from 1 to 4 hetero atoms selected from the group consisting of nitrogen, sulfur or oxygen within the ring wherein, in fused ring systems, one or more the rings may be cycloalkyl, aryl or heteroaryl provided that the point of attachment is through the non-aromatic ring. In one embodiment, the nitrogen and/or sulfur atom(s) of the heterocyclic group are optionally oxidized to provide for the N-oxide, sulfinyl, sulfonyl moieties.

As used herein, “sulfo” refers to sulfonic acid or sulfonate.

As used herein, “halogen” refers to fluorine, chlorine, bromine, or iodine.

Each of the above terms (e.g., “alkyl,” “heteroalkyl,” “aryl” and “heteroaryl”) includes both substituted and unsubstituted forms of the indicated radical.

As used herein, the term “heteroatom” includes oxygen (O), nitrogen (N), sulfur (S), phosphorous (P) and selenium (Se).

The term “amino” or “amine group” refers to the group —NR′R″ (or —N⁺RR′R″) where R, R′ and R″ are independently selected from the group consisting of hydrogen, alkyl, substituted alkyl, aryl, substituted aryl, aryl alkyl, substituted aryl alkyl, heteroaryl, and substituted heteroaryl. A substituted amine being an amine group wherein R′ or R″ is other than hydrogen. In a primary amino group, both R′ and R″ are hydrogen, whereas in a secondary amino group, either, but not both, R′ or R″ is hydrogen. In addition, the terms “amine” and “amino” can include protonated and quaternized versions of nitrogen, comprising the group —N⁺RR′R″ and its biologically compatible anionic counterions.

The term “sample” as used herein refers to any material that may contain nucleic acid. The sample may also include diluents, buffers, detergents, and contaminating species, debris and the like that are found mixed with the target. Illustrative examples include urine, sera, blood plasma, total blood, saliva, tear fluid, cerebrospinal fluid, secretory fluids and the like. Also included are solid, gel or substances such as mucus, body tissues, cells and the like suspended or dissolved in liquid materials such as buffers, extractants, solvents and the like. Typically, the sample is a live cell, a biological fluid that comprises endogenous host cell proteins, nucleic acid polymers, nucleotides, nucleosides, oligonucleotides, peptides and buffer solutions. The sample may be in an aqueous solution, a viable cell culture or immobilized on a solid or semi solid surface such as a polyacrylamide gel, membrane blot or on a microarray.

As used herein, “microRNA” or “miRNA” as used herein refers to a small ribonucleic acid (RNA) molecule consisting of about 25 or fewer nucleotides. Typical miRNA molecules have a length of between about 21 to 25 nucleotides. MicroRNAs are highly conserved RNA molecules that modulate the expression of their cognate mRNA molecules and prevent gene expression.

As used herein, “dye” refers to a compound that emits light to produce an observable detectable signal.

The term “detectable signal” as used herein refers to a change in or an occurrence of, a signal that is directly or indirectly detectable either by observation or by instrumentation. Typically, the detectable signal is an optical response resulting in a change in the wavelength distribution patterns or intensity of absorbance or fluorescence or a change in light scatter, fluorescence lifetime, fluorescence polarization, or a combination of the above parameters.

As used herein, the term “kit” refers to a packaged set of related components, typically one or more compounds or compositions. The kits disclosed herein comprise one or more of the cyanine dye compounds described herein, one or more carriers suitable for in vitro applications, and one or more containers in which to store the one or more dyes and/or one or more carriers, such as solvents, buffers, stabilizers, pH adjusting agents, etc. The kit optionally contains instructions for how to use and detect the dye or composition containing the dye in an assay for detection of ssRNA in a sample. The kit can further contain one or more pieces of equipment to administer the dye, or composition containing the dye including, but not limited to, syringes, pipettes, pipette bulbs, spatulas, vials, syringe needles, and various combinations thereof.

The Compounds:

Disclosed herein are substituted unsymmetrical cyanine dyes and methods for their use in the detection of RNA, such as short and/or single-stranded RNA, as well as compositions, kits, including substituted unsymmetrical cyanine dyes. The compounds, compositions, methods, and kits can be used to detect RNA, for example, by conventional fluorescence microscopy, fluorescence spectroscopy, flow cytometry, and/or high content imaging.

The dye compounds disclosed herein, and compositions containing those dyes, can be used for a variety of in vitro or ex vivo assays designed for the detection of RNA molecules, e.g., short and/or single-stranded RNA molecules. For example, the dyes can be used for single cell imaging or to assay a cell suspension, during which the dye(s) are loaded into cells by incubation with the cells for a sufficient period of time. Specific assays include, but are not limited to, those with live organ cultures as well as cell culture assays.

The disclosed dyes and compositions can be used advantageously for the detection of short and/or single-stranded RNA molecules. It was surprisingly found that the dye compounds and the methods disclosed herein can be used to detect short, single-stranded RNA molecules that are usually not detectable by standard fluorescent methods. Furthermore, the methods provided herein can detect very low concentrations of short RNA molecules even in the presence of larger RNA molecules such as rRNA and mRNA. It was also surprisingly found that the compositions and methods provided herein are more specific for short RNA molecules than for larger RNA molecules. The compositions and methods provided herein overcome many of the disadvantages associated with conventional double-stranded nucleic acid binding dyes and provide new opportunities for the use in the analysis and quantification of RNA.

The dye compounds of the present teachings comprise: 1) a first heterocyclic ring system that is a substituted benzazolium ring, 2) a bridging methine, and 3) a second heterocyclic ring that is a quinolinium ring system, one or more positions of which may be substituted by a TAIL that contains at least one heteroatom. The first and second ring systems are optionally further substituted by a variety of substituents as described below.

TAIL

TAIL is a heteroatom-containing side chain that is described by the formula LINK-SPACER-CAP. LINK is the linking moiety by which TAIL is attached to the core structure of the dye compounds disclosed herein. SPACER is a covalent linkage that connects LINK to CAP. CAP is the portion of TAIL that possesses a heteroatom component.

LINK is a single covalent bond, an ether linkage (—O—), a thioether linkage (—S—), or an amine linkage (—NR₂—). In each embodiment, LINK forms the attachment between the dye core structure and SPACER. When LINK is an amine, the amine substituent (R₂₀) is optionally H, such that LINK-NH—. Alternatively, R₂₀ is a linear or branched alkyl having 1-8 carbons. In another embodiment, R₂₀ is -SPACER′-CAP′, yielding a TAIL having the formula:

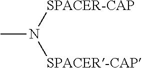

where SPACER′ and CAP′, respectively, may be the same as or different from SPACER and CAP, and are selected from the same alternatives defined for SPACER and CAP, respectively. For the sake of simplifying the description, SPACER and CAP are defined with the understanding that a description of SPACER includes SPACER′ and a description of CAP includes CAP′.

SPACER is a covalent linkage that joins LINK and CAP. SPACER is a linear, branched, cyclic, heterocyclic, saturated or unsaturated arrangement of 1-16 carbon (C), nitrogen (N), phosphorous (P), oxygen (O) or sulfur (S) atoms. Alternatively, SPACER is a single covalent bond, such that both LINK and SPACER are not simultaneously single covalent bonds. Preferably, the SPACER linkage must begin and end with a carbon atom. Typically, if SPACER consists of a single atom, it is required to be a carbon atom, so that the first and last atom in SPACER (in this specific instance, they are the same atom) is a carbon. The 1-16 atoms making up SPACER are combined using any appropriate combination of ether, thioether, amine, ester, or amide bond; or single, double, triple or aromatic carbon-carbon bonds; or phosphorous-oxygen bonds; or phosphorus-sulfur bonds; or nitrogen-nitrogen bonds; or nitrogen-oxygen bonds; or aromatic or heteroaromatic bonds. SPACER is further substituted by hydrogen to accommodate the valence state of each atom in SPACER.

Generally, the atoms of SPACER are arranged such that all heteroatoms in the linear backbone of SPACER are separated by at least one carbon atom, and preferably separated by at least two carbon atoms. Typically, SPACER is 1-6 carbon atoms in a linear or branched saturated chain. In one embodiment, SPACER incorporates a 6-membered heteroaromatic ring (phenylene linkage). In another embodiment, SPACER incorporates a 5- or 6-membered heteroaromatic ring, wherein the heteroatoms are O, N, or S. Alternatively, SPACER incorporates amide linkages, ester linkages, simple ethers and thioethers, and amines in a linear arrangement, such as —CH₂—CH₂—(C═O)—NH—CH₂—CH₂—CH₂—. Preferably, SPACER is an alkylene (—(CH₂)ₖ—, where k=1-8).

LINK and SPACER, in combination, serve to attach a heteroatom-containing group, CAP, to the dye core structure. CAP may contain oxygen, sulfur, or nitrogen, according to the formulae —OR₂₁, —SR₂₁, —NR₂₁R₂₂, or —N⁺R₂₁R₂₂R₂₃Ψ⁻. The substituents R₂₁, R₂₂ and R₂₃ are independently H, or a linear or branched alkyl or cycloalkyl having 1-8 carbons. Where any of R₂₁, R₂₂ and R₂₃ are alkyl or cycloalkyl, they are optionally further substituted by halogen, hydroxyl, alkoxy having 1-8 carbons, amino, carboxy, or phenyl, where phenyl is optionally further substituted by halogen, hydroxyl, alkoxy having 1-8 carbons, amino, aminoalkyl having 1-8 carbons, or carboxyalkyl having 1-8 carbons. In another embodiment, one or more of R₂₁, R₂₂ and R₂₃, taken in combination with SPACER forms a 5- or 6-membered ring that is aromatic, heteroaromatic, alicyclic or heteroalicyclic ring. When the 5- or 6-membered ring is heteroaromatic or heteroalicyclic, the ring contains 1-3 heteroatoms that are O, N or S. Alternatively, one or more of R₂₁, R₂₂, and R₂₃, taken in combination with R₂₀ and SPACER, forms a 5- or 6-membered ring that is aromatic, heteroaromatic, alicyclic or heteroalicyclic, as described above. Preferably, R₂₁ and R₂₂ are hydrogen, or alkyl having 1-8 carbons. R₂₃ is typically H or alkyl having 1-8 carbons.

When CAP is —N⁺R₂₁R₂₂R₂₃Ψ⁻, the biologically compatible counter ion balances the positive charge present on the CAP nitrogen, which is a quaternary ammonium salt. As used herein, a substance that is biologically compatible is not toxic as used, and does not have a substantially deleterious effect on biomolecules. Examples of Ψ⁻ include, among others, chloride, bromide, iodide, sulfate, alkanesulfonate, arylsulfonate, phosphate, perchlorate, tetrafluoroboronate, tetraarylboride, nitrate, and anions of aromatic or aliphatic carboxylic acids. Preferred ψ-counterions are bromide, chloride, iodide, perchlorate, and various sulfonates.

Additionally, there are several embodiments wherein CAP incorporates a cyclic structure. In these embodiments, CAP typically incorporates a 5- or 6-membered nitrogen-containing ring, optionally including an additional heteroatom (typically oxygen), where the ring nitrogen is optionally substituted by R₂₃ to give an ammonium salt. Specific versions of CAP include, but are not limited to,

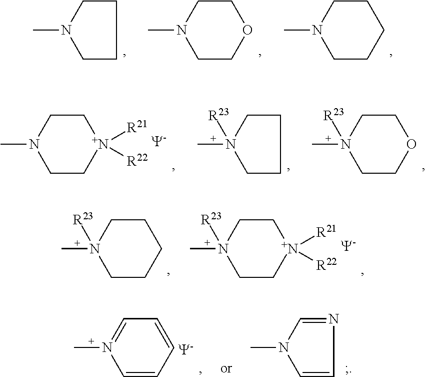

CAP is preferably —NR₂₁R₂₂ or —N+R₂₁R₂₂R₂₃Ψ⁻, where R₂₁, R₂₂, and R₂₃ are alkyls having 1-6 carbons. More preferably, CAP is —N(CH₃)₂ or —N⁺(CH₃)₃Ψ⁻.

Preferably, TAIL contains 6-10 non-hydrogen atoms, including LINK and CAP. Selected examples of TAIL are listed in Table 1. For each TAIL, the identities of LINK, SPACER and CAP are specified. Where R₂₁, R₂₂, or R₂₃ combined with either R₂₀ or SPACER, the combination is indicated in this table.

TABLE 1
Specific Examples of TAIL Moieties

| TAIL | LINK | SPACER/SPACER′ | CAP/CAP′ |
|---|---|---|---|
| 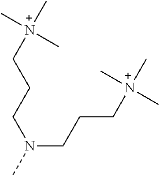 | —N—SPACER—CAP —N—SPACER′—CAP′ | —CH₂CH₂CH₂— —CH₂CH₂CH₂— | —N+(CH₃)₃ —N+(CH₃)₃ |
| 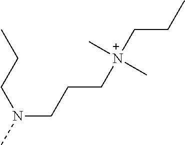 | —N(CH₂CH₂CH₃)— | —CH₂CH₂CH₂— | —N⁺(CH₃)₂[(CH₂)₂CH₃] |
| 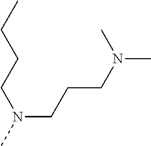 | —N(CH₂CH₂CH₂CH₃)— | —CH₂CH₂CH₂— | —N(CH₃)₂ |
| 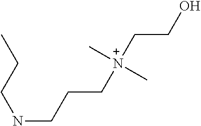 | —N(CH₂CH₂CH₃)— | —CH₂CH₂CH₂— | —N⁺(CH₃)₂[(CH₂)₂OH] |
| 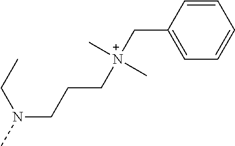 | —N(CH₂CH₃)— | —CH₂CH₂CH₂— | —N⁺(CH₃)₂[(CH₂)—phenyl] |
| 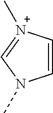 | —NR²³— | —CH═N—CH═CH— | 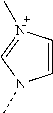 |

In the methods provided herein, the sample suspected of containing a short RNA molecule (e.g., microRNA) is combined with a substituted unsymmetrical cyanine dye compound. An exemplary substituted, unsymmetrical cyanine dye compound is represented by the structure shown in Formula I:

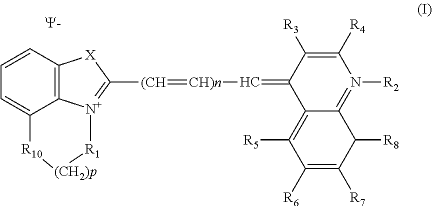

wherein
X is O or S;
n is 0, 1 or 2;
p is 0, 1 or 2;
Ψ is a biologically compatible counterion;
R₁ and R₁₀, which can be the same or different, are independently H; or a substituted or unsubstituted alkyl group (e.g., an alkyl group having 1-6 carbons), wherein the substitution on the alkyl group, if present, is a carboxyl or (alkyl)ammonium group;
R₂ is a substituted or unsubstituted alkyl, naphthyl, phenyl, thienyl, or cycloalkyl having 3-8 carbons; wherein the substitution on R₂, if present, is a carboxyl or carboxamide group;
R₃ is H; or an alkyl, alkenyl, polyalkenyl, alkynyl or polyalkynyl group (e.g., having 1-6 carbons); or a halogen; or a substituted or unsubstituted aryl or heteroaryl; or a substituted or unsubstituted cycloalkyl having 3-10 carbons; or —OR₁₁, —SR₁₁, —(NR₉R₁₁); or —OSO₂R₁₉; or a TAIL;
wherein R₉ and R₁₁, which can be the same or different, are independently H; or substituted or unsubstituted alkyl groups (e.g., alkyl groups having 1-6 carbons); or alkyl groups substituted with one or more alkylammonium groups; or 1-2 alicyclic or aromatic rings; or R₉ and R₁₁ taken in combination are —(CH₂)₄— or —(CH₂)₅— to give a 5 or 6 membered ring; and where R₁₉ is alkyl (e.g., having 1-6 carbons), or perfluoroalkyl (e.g., having 1-6 carbons), or aryl;
R₄ is a substituted or unsubstituted alkyl, a substituted or unsubstituted alkenyl, a substituted or unsubstituted polyalkenyl, a substituted or unsubstituted alkynyl or a substituted or unsubstituted polyalkynyl group (e.g., having 1-6 carbons); or a halogen; or a substituted or unsubstituted aryl or heteroaryl; or a substituted or unsubstituted cycloalkyl having 3-10 carbons; or —OR₁₁, —SR₁₁, —(NR₉R₁₁); or —OSO₂R₁₉; or a TAIL;
R₅, R₆, R₇ and R₈, which can be the same or different, are independently H; or an alkyl, alkenyl, polyalkenyl, alkynyl or polyalkynyl group (e.g., having 1-6 carbons); or a halogen; or a substituted or unsubstituted aryl or heteroaryl; or a substituted or unsubstituted cycloalkyl having 3-8 carbons; or a TAIL; or —OR₁₁, —SR₁₁, or —(NR₉R₁₁);
TAIL is a heteroatom-containing moiety having the formula LINK-SPACER-CAP;
wherein
LINK is a single covalent bond, —O—, —S—, or —NR₂₀; where R₂₀ is H, a linear or branched alkyl having 1-8 carbons, or R₂₀ is -SPACER′-CAP′;
SPACER and SPACER′, which can be the same or different, are covalent linkages, linear or branched, cyclic or heterocyclic, saturated or unsaturated, each having 1-16 nonhydrogen atoms selected from the group consisting of C, N, O and S, such that the linkage begins and ends with a carbon atom, and contains any combination of ether, thioether, amine, ester, amide, or aliphatic, oleic or aromatic carbon-carbon bonds, or aromatic carbon-nitrogen or nitrogen-nitrogen bonds; wherein all heteroatoms in the linear backbone of SPACER are separated by at least two carbon atoms;
CAP and CAP′, which can be the same or different, are —OR₂₁, —SR₂₁, —NR₂₁R₂₂, or —N⁺R₂₁R₂₂R₂₃Ψ⁻;
wherein
R₂₁, R₂₂, and R₂₃ are independently H, or a linear or branched alkyl or cycloalkyl having 1-8 carbons, optionally further substituted by halogen, hydroxy, alkoxy having 1-8 carbons, carboxyalkyl having 1-8 carbons, or phenyl, where phenyl is optionally further substituted by halogen, hydroxy, alkoxy having 1-8 carbons, aminoalkyl having 1-8 carbons, or carboxyalkyl having 1-8 carbons; or, one or more of R₂₁, R₂₂, and R₂₃, taken in combination with SPACER or SPACER′ or R₂₀ forms a 5- or 6-membered aromatic, heteroaromatic, alicyclic or heteroalicyclic ring, the heteroatoms selected from O, N or S; where Ψ⁻ is a biologically compatible counterion; or
CAP and CAP′ are independently

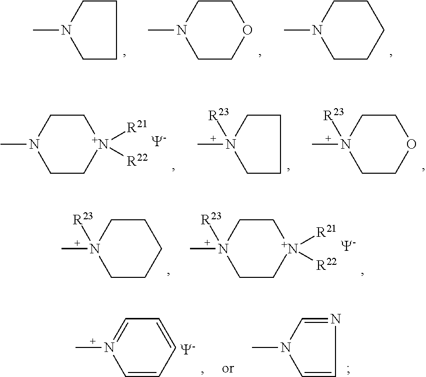

where R₂₁, R₂₂, R₂₃ and Ψ⁻ are as defined previously; such that at least one of R₃, R₄, R₅, R₆, R₇, R₈ or R₁₀ is a TAIL; and, where more than one substituent is a TAIL, each TAIL is optionally the same or different;
or wherein at least one of R₁, R₅, R₆, R₇, R₈, R₉, R₁₀, R₁₁ or R₂₀, R₂₁, R₂₂ or R₂₃ is a water-soluble group.

When CAP is —N⁺R₂₁R₂₂R₂₃Ψ⁻, the biologically compatible counter ion balances the positive charge present on the CAP nitrogen, which is a quaternary ammonium salt. As used herein, a substance that is biologically compatible is not toxic as used, and does not have a substantially deleterious effect on biomolecules. Examples of Ψ⁻ include, among others, chloride, bromide, iodide, sulfate, alkanesulfonate, arylsulfonate, phosphate, perchlorate, tetrafluoroboronate, tetraarylboride, nitrate, and anions of aromatic or aliphatic carboxylic acids. Preferred ψ-counterions are bromide, chloride, iodide, perchlorate, and various sulfonates.

In certain embodiments, if p is 0, R₁ and R₁₀, which can be the same or different, are independently H; or a substituted or unsubstituted alkyl group (e.g., an alkyl group having 1-6 carbons), wherein the substitution on the alkyl group, if present, is a carboxyl or (alkyl)ammonium group.

In certain embodiments, if p is not 0, R₁ and R₁₀, which can be the same or different, are independently a substituted or unsubstituted alkyl group (e.g., an alkyl group having 1-6 carbons), wherein the substitution on the alkyl group, if present, is a carboxyl or (alkyl)ammonium group.

The water-soluble group can include a polymer (e.g., a polyalkylene oxide, carbohydrate and polypeptide). In certain embodiments, the water-soluble group comprises a polyethylene oxide. In certain embodiments, R₄ is a TAIL; R₂ is a substituted or unsubstituted phenyl; and/or at least one of R₅, R₆, R₇ and R₈ is —OR₁₁, —SR₁₁, or —NR₉R₁₁). In certain embodiments, n is 0. In certain embodiments, at least one of R₅, R₆, R₇ and R₈ is —OR₁₁. In certain embodiments, OR₁₁ is —OCH₃. In certain embodiments, R₇ is —OCH₃. In certain embodiments, R₁ is an unsubstituted alkyl and R₁₀ is H. In certain embodiments, R₁ is methyl and R₁₀ is H.

In certain embodiments, the dye compound of Formula I is selected from the group consisting of Compound 4, Compound 5, Compound 6, Compound 7, Compound 8, Compound 9, Compound 10, Compound 11, Compound 12, Compound 13, Compound 14, Compound 15, Compound 16, Compound 17, Compound 18, Compound 19, Compound 20, Compound 21, Compound 23, Compound 25, Compound 26, Compound 27, Compound 30, Compound 31, Compound 32, Compound 33, Compound 34, Compound 35, Compound 36, Compound 37, Compound 38, Compound 39, Compound 40, Compound 41, Compound 42, Compound 43, Compound 44, Compound 45, Compound 46, Compound 47, Compound 48, Compound 49, Compound 50, Compound 51, Compound 52, Compound 53, Compound 54, Compound 55, Compound 56, Compound 57, Compound 58, Compound 59, Compound 60, Compound 61, Compound 62, Compound 63, Compound 64, and Compound 65.

In certain embodiments, the substituted, unsymmetrical cyanine dye compound, includes a first heterocyclic ring system that is a substituted benzothiazolium, benzoxazolium, benzoselenazolium, benzimidazolium, or dialkylindolinium, wherein the first ring system is linked by a monomethine or trimethine bridging moiety attached at the 2-position of the first ring system to the 2- or 4-position of a second heterocyclic ring system that is a substituted quinolinium or that is linked by a monomethine bridging moiety attached to the 2-position of a second heterocyclic ring system that is a substituted pyridinium; wherein one or more substituents of the second ring system is a substituted or unsubstituted cyclohexyl, cyclohexenyl, morpholino, piperidinyl, naphthyl, phenyl, thienyl, benzothizolyl, furanyl, oxazolyl, benzoxazolyl or pyridinyl group.

The cyanine compounds disclosed herein optionally can include one or more groups that can enhance the solubility of the dye in an aqueous medium. Such groups are referred to herein as “water soluble groups” (WSG). In certain embodiments, such groups can include a water-soluble polymer. Various types of water-soluble polymers can be used as a WSG on the dyes disclosed herein. Typically, the water-soluble polymer group has a molecular weight of about 300 Da to 3000 Da and can be a charged, zwitterionic, or neutral compound. Representative examples of suitable water-soluble polymer groups include, without limitation, polyalkylene oxides, carbohydrates and polypeptides. In certain embodiments, the water-soluble polymer group is a polyethylene oxide.

Certain dye embodiments described by Formula I are particularly sensitive to the presence of relatively short ssRNA sequences (e.g., less than 25 nucleotides in length) when used in the methods provided herein. Representative examples of such dyes include those in which R₄ is a TAIL or —(NR₉R₁₁); R₂ is a substituted or unsubstituted phenyl; and/or at least one of R₅, R₆, R₇ and R₈ is —OR₁₁, —SR₁₁, or —(NR₉R₁₁). In certain embodiments, n is 0. In certain embodiments, at least one of R₅, R₆, R₇ and R₈ is —OR₁₁. In certain embodiments, OR₁₁ is —OCH₃. In certain embodiments, R₇ is OCH₃. In certain embodiments, R₁ is an unsubstituted alkyl and R₁₀ is H. In certain embodiments, R₁ is methyl and R₁₀ is H.

A specific substituted unsymmetrical cyanine dye compound that can be particularly useful for detecting relatively short sequences of RNA (e.g., miRNA) in the disclosed methods is represented by the structure shown in Formula II:

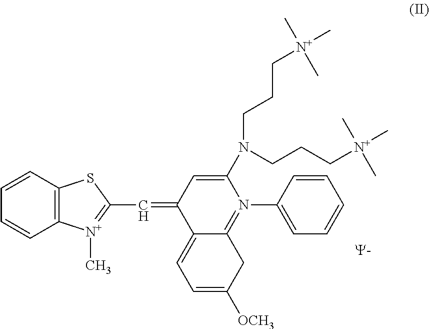

Further examples of substituted unsymmetrical cyanine dye compound for use in the disclosed methods are described in U.S. Pat. No. 5,658,751, which is herein incorporated by reference in its entirety.

It was surprisingly found that dyes of the present teachings that comprise a quaternary ammonium salt derived from either a benzyl or a 3-methylbenzyl group have increased fluorescent enhancement, as compared to the dye of Formula II, upon or after interacting with RNA. Without wishing to be bound by theory, the dye compounds of the present teachings with increased fluorescent enhancement comprise two positive charges: one from the benzothiazolium and the other from the tetraalkylammonium salt. Furthermore, the benzyl or 3-methylbenzyl group that forms the quaternary ammonium salt appears to have an effect on the hydrophobicity and/or hydrophilicity of the dye compounds disclosed herein.

The methods provided herein typically involve formation of a fluorescent complex if the sample contains a certain quantity of RNA. The association between the RNA and the dye compounds provided herein can involve a covalent or non-covalent (e.g., ionic) interaction. In certain embodiments, the cyanine dyes disclosed herein bind to RNA non-covalently.

In yet another aspect, a fluorescent complex is provided that includes ssRNA, wherein the ssRNA comprises 25 or fewer nucleotides, non-covalently bound to a dye of the Formula II:

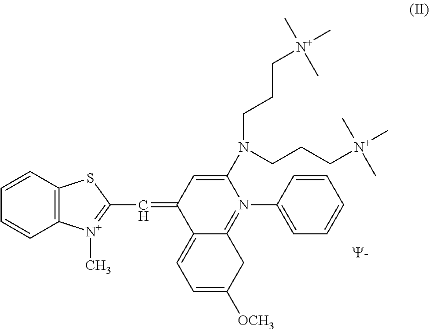

wherein the complex emits a detectable fluorescent signal.

In yet another aspect, a fluorescent complex is produced that includes ssRNA, wherein the ssRNA comprises 25 or fewer nucleotides, non-covalently bound to a dye of the Formula I:

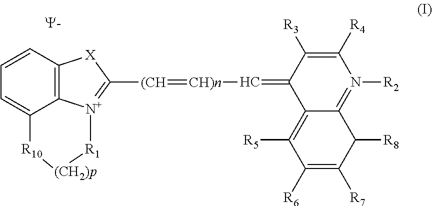

w wherein
X is O or S;
n is 0, 1 or 2;
p is 0, 1 or 2;
Ψ is a biologically compatible counterion;

R₁ and R₁₀, which can be the same or different, are independently H; or a substituted or unsubstituted alkyl group, wherein the substitution on the alkyl group, if present, is a carboxyl or (alkyl)ammonium group;
R₂ is a substituted or unsubstituted alkyl, naphthyl, phenyl, thienyl, or cycloalkyl having 3-8 carbons, wherein the substitution on R₂, if present, is a carboxyl or carboxamide group;
R₃ is H; or an alkyl, alkenyl, polyalkenyl, alkynyl or polyalkynyl group; or a halogen; or a substituted or unsubstituted aryl or heteroaryl; or a substituted or unsubstituted cycloalkyl having 3-10 carbons; or —OR₁₁, —SR₁₁, —(NR₉R₁₁); or —OSO₂R₁₉; or a TAIL;
wherein R₉ and R₁₁, which can be the same or different, are independently H; or substituted or unsubstituted alkyl groups; or alkyl groups substituted with one or more alkylammonium groups; or 1-2 alicyclic or aromatic rings; or R₉ and R₁₁ taken in combination are —(CH₂)₄— or —(CH₂)₅— to give a 5 or 6 membered ring; and where R₁₉ is alkyl, or perfluoroalkyl, or aryl;
R₄ is an alkyl, alkenyl, polyalkenyl, alkynyl or polyalkynyl group; or a halogen; or a substituted or unsubstituted aryl or heteroaryl; or a substituted or unsubstituted cycloalkyl having 3-10 carbons; or —OR₁₁, —SR₁₁, —(NR₉R₁₁); or —OSO₂R₁₉; or a TAIL;
R₅, R₆, R₇ and R₈, which can be the same or different, are independently H; or an alkyl, alkenyl, polyalkenyl, alkynyl or polyalkynyl group; or a halogen; or a substituted or unsubstituted aryl or heteroaryl; or a substituted or unsubstituted cycloalkyl having 3-8 carbons; or a TAIL; or —OR₁₁, —SR₁₁, or —(NR₉R₁₁);
TAIL is a heteroatom-containing moiety having the formula LINK-SPACER-CAP;
wherein
LINK is a single covalent bond, —O—, —S—, or —NR₂₀; where R₂₀ is H, a linear or branched alkyl having 1-8 carbons, or R₂₀ is -SPACER′-CAP′;
SPACER and SPACER′, which can be the same or different, are covalent linkages, linear or branched, cyclic or heterocyclic, saturated or unsaturated, each having 1-16 nonhydrogen atoms selected from the group consisting of C, N, O and S, such that the linkage begins and ends with a carbon atom, and contains any combination of ether, thioether, amine, ester, amide, or aliphatic, oleic or aromatic carbon-carbon bonds, or aromatic carbon-nitrogen or nitrogen-nitrogen bonds; wherein all heteroatoms in the linear backbone of SPACER are separated by at least two carbon atoms;
CAP and CAP′, which can be the same or different, are —OR₂₁, —SR₂₁, —NR₂₁R₂₂, or —N⁺R₂₁R₂₂R₂₃Ψ⁻;
wherein
R₂₁, R₂₂, and R₂₃ are independently H, or a linear or branched alkyl or cycloalkyl having 1-8 carbons, optionally further substituted by halogen, hydroxy, alkoxy having 1-8 carbons, carboxyalkyl having 1-8 carbons, or phenyl, where phenyl is optionally further substituted by halogen, hydroxy, alkoxy having 1-8 carbons, aminoalkyl having 1-8 carbons, or carboxyalkyl having 1-8 carbons; or, one or more of R₂₁, R₂₂, and R₂₃, taken in combination with SPACER or SPACER′ or R₂₀ forms a 5- or 6-membered aromatic, heteroaromatic, alicyclic or heteroalicyclic ring, the heteroatoms selected from O, N or S; where Ψ⁻ is a biologically compatible counterion; or
CAP and CAP′ are independently

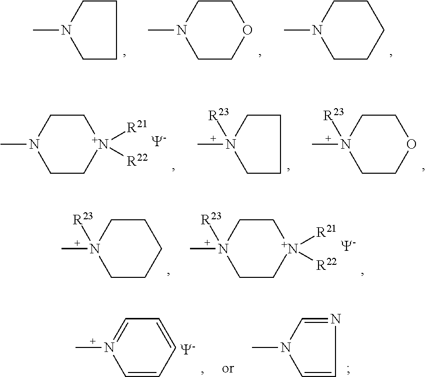

where R₂₁, R₂₂, R₂₃ and Ψ⁻ are as defined previously; such that at least one of R₃, R₄, R₅, R₆, R₇, R₈ or R₁₀ is a TAIL; and, where more than one substituent is a TAIL, each TAIL is optionally the same or different; or
or wherein at least one of R₁, R₅, R₆, R₇, R₈, R₉, R₁₀, R₁₁ or R₂₀, R₂₁, R₂₂ or R₂₃ is a water-soluble group (WSG);

wherein the complex emits a detectable fluorescent signal.

When CAP is —N⁺R₂₁R₂₂R₂₃Ψ⁻, the biologically compatible counter ion Ψ⁻ balances the positive charge present on the CAP nitrogen, which is a quaternary ammonium salt. As used herein, a substance that is biologically compatible is not toxic as used, and does not have a substantially deleterious effect on biomolecules. Examples of Ψ⁻ include, among others, chloride, bromide, iodide, sulfate, alkanesulfonate, arylsulfonate, phosphate, perchlorate, tetrafluoroboronate, tetraarylboride, nitrate, and anions of aromatic or aliphatic carboxylic acids. Preferred ψ-counterions are bromide, chloride, iodide, perchlorate, and various sulfonates

The water-soluble group can include a polymer (e.g., a polyalkylene oxide, carbohydrate and polypeptide). In certain embodiments, the water-soluble group comprises a polyethylene oxide. In certain embodiments, R₄ is a TAIL; R₂ is a substituted or unsubstituted phenyl; and/or at least one of R₅, R₆, R₇ and R₈ is —OR₁₁, —SR₁₁, or —NR₉R₁₁). In certain embodiments, n is 0. In certain embodiments, at least one of R₅, R₆, R₇ and R₈ is —OR₁₁. In certain embodiments, —OR₁₁ is —OCH₃. In certain embodiments, R₇ is —OCH₃. In certain embodiments, R₁ is an unsubstituted alkyl and R₁₀ is H. In certain embodiments, R₁ is methyl and R₁₀ is H.

In certain embodiments, wherein if p is 0, and wherein R₁ and R₁₀, which can be the same or different, are independently H; or a substituted or unsubstituted alkyl group, wherein the substitution on the alkyl group, if present, is a carboxyl or (alkyl)ammonium group. In other embodiments, wherein if p is not 0, R₁ and R₁₀, which can be the same or different, are independently a substituted or unsubstituted alkyl group, wherein the substitution on the alkyl group, if present, is a carboxyl or (alkyl)ammonium group.

In certain embodiments, the dye compound of Formula I is selected from the group consisting of Compound 4, Compound 5, Compound 6, Compound 7, Compound 8, Compound 9, Compound 10, Compound 11, Compound 12, Compound 13, Compound 14, Compound 15, Compound 16, Compound 17, Compound 18, Compound 19, Compound 20, Compound 21, Compound 23, Compound 25, Compound 26, Compound 27, Compound 30, Compound 31, Compound 32, Compound 33, Compound 34, Compound 35, Compound 36, Compound 37, Compound 38, Compound 39, Compound 40, Compound 41, Compound 42, Compound 43, Compound 44, Compound 45, Compound 46, Compound 47, Compound 48, Compound 49, Compound 50, Compound 51, Compound 52, Compound 53, Compound 54, Compound 55, Compound 56, Compound 57, Compound 58, Compound 59, Compound 60, Compound 61, Compound 62, Compound 63, Compound 64, and Compound 65.

In yet another aspect, a fluorescent complex is produced that includes ssRNA, wherein the ssRNA comprises 25 or fewer nucleotides, non-covalently bound to a dye compound selected from the group consisting of Compound 4, Compound 5, Compound 6, Compound 7, Compound 8, Compound 9, Compound 10, Compound 11, Compound 12, Compound 13, Compound 14, Compound 15, Compound 16, Compound 17, Compound 18, Compound 19, Compound 20, Compound 21, Compound 23, Compound 25, Compound 26, Compound 27, Compound 30, Compound 31, Compound 32, Compound 33, Compound 34, Compound 35, Compound 36, Compound 37, Compound 38, Compound 39, Compound 40, Compound 41, Compound 42, Compound 43, Compound 44, Compound 45, Compound 46, Compound 47, Compound 48, Compound 49, Compound 50, Compound 51, Compound 52, Compound 53, Compound 54, Compound 55, Compound 56, Compound 57, Compound 58, Compound 59, Compound 60, Compound 61, Compound 62, Compound 63, Compound 64, and Compound 65, wherein the complex emits a detectable signal.

In yet another aspect, a fluorescent complex is produced that includes ssRNA, wherein the ssRNA comprises 25 or fewer nucleotides, non-covalently bound to a substituted, unsymmetrical cyanine dye compound, wherein the dye comprises a first heterocyclic ring system that is a substituted benzothiazolium, benzoxazolium, benzoselenazolium, benzimidazolium, or dialkylindolinium, wherein the first ring system is linked by a monomethine or trimethine bridging moiety attached at the 2-position of the first ring system to the 2- or 4-position of a second heterocyclic ring system that is a substituted quinolinium or that is linked by a monomethine bridging moiety attached to the 2-position of a second heterocyclic ring system that is a substituted pyridinium; wherein one or more substituents of the second ring system is a substituted or unsubstituted cyclohexyl, cyclohexenyl, morpholino, piperidinyl, naphthyl, phenyl, thienyl, benzothizolyl, furanyl, oxazolyl, benzoxazolyl or pyridinyl group, wherein the complex emits a detectable signal.

The fluorescent signal generated by the fluorescent complex is extremely stable. For example, the signal can be stable for at least 30 minutes at room temperature. In some embodiments, the fluorescence signal is stable for at least 3 hours at room temperature.

Also provided herein are compositions for the detection of single-stranded RNA compounds. Such compositions can include a) cyanine dye compounds, as described herein; and b) a carrier, wherein the dye compounds are present in an amount effective to detect the presence of ssRNA in the sample.

In certain aspects, the compositions provided herein include dye compounds comprising the structure of Formula I:

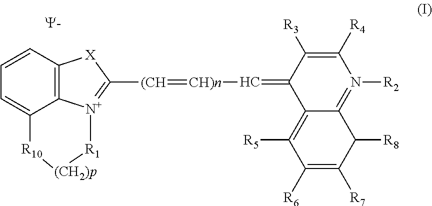

wherein
X is O or S;
n is 0, 1 or 2;
p is 0, 1 or 2;
Ψ is a biologically compatible counterion;
R₁ and R₁₀, which can be the same or different, are independently H; or a substituted or unsubstituted alkyl group, wherein the substitution on the alkyl group, if present, is a carboxyl or (alkyl)ammonium group;
R₂ is a substituted or unsubstituted alkyl, naphthyl, phenyl, thienyl, or cycloalkyl having 3-8 carbons, wherein the substitution on R₂, if present, is a carboxyl or carboxamide group;
R₃ is H; or an alkyl, alkenyl, polyalkenyl, alkynyl or polyalkynyl group; or a halogen; or a substituted or unsubstituted aryl or heteroaryl; or a substituted or unsubstituted cycloalkyl having 3-10 carbons; or —OR₁₁, —SR₁₁, —(NR₉R₁₁); or —OSO₂R₁₉; or a TAIL;
wherein R₉ and R₁₁, which can be the same or different, are independently H; or substituted or unsubstituted alkyl groups; or alkyl groups substituted with one or more alkylammonium groups; or 1-2 alicyclic or aromatic rings; or R₉ and R₁₁ taken in combination are —(CH₂)₄— or —(CH₂)₅— to give a 5 or 6 membered ring; and where R₁₉ is alkyl, or perfluoroalkyl, or aryl;
R₄ is an alkyl, alkenyl, polyalkenyl, alkynyl or polyalkynyl group; or a halogen; or a substituted or unsubstituted aryl or heteroaryl; or a substituted or unsubstituted cycloalkyl having 3-10 carbons; or —OR₁₁, —SR₁₁, —(NR₉R₁₁); or —OSO₂R₁₉; or a TAIL;
R₅, R₆, R₇ and R₈, which can be the same or different, are independently H; or an alkyl, alkenyl, polyalkenyl, alkynyl or polyalkynyl group; or a halogen; or a substituted or unsubstituted aryl or heteroaryl; or a substituted or unsubstituted cycloalkyl having 3-8 carbons; or a TAIL; or —OR₁₁, —SR₁₁, or —(NR₉R₁₁);
TAIL is a heteroatom-containing moiety having the formula LINK-SPACER-CAP;
wherein
LINK is a single covalent bond, —O—, —S—, or —NR₂₀; where R₂₀ is H, a linear or branched alkyl having 1-8 carbons, or R₂₀ is -SPACER′-CAP′;
SPACER and SPACER′, which can be the same or different, are covalent linkages, linear or branched, cyclic or heterocyclic, saturated or unsaturated, each having 1-16 nonhydrogen atoms selected from the group consisting of C, N, O and S, such that the linkage begins and ends with a carbon atom, and contains any combination of ether, thioether, amine, ester, amide, or aliphatic, oleic or aromatic carbon-carbon bonds, or aromatic carbon-nitrogen or nitrogen-nitrogen bonds; wherein all heteroatoms in the linear backbone of SPACER are separated by at least two carbon atoms;
CAP and CAP′, which can be the same or different, are —OR₂₁, —SR₂₁, —NR₂₁R₂₂, or —N⁺R₂₁R₂₂R₂₃Ψ⁻;
wherein
R₂₁, R₂₂, and R₂₃ are independently H, or a linear or branched alkyl or cycloalkyl having 1-8 carbons, optionally further substituted by halogen, hydroxy, alkoxy having 1-8 carbons, carboxyalkyl having 1-8 carbons, or phenyl, where phenyl is optionally further substituted by halogen, hydroxy, alkoxy having 1-8 carbons, aminoalkyl having 1-8 carbons, or carboxyalkyl having 1-8 carbons; or, one or more of R₂₁, R₂₂, and R₂₃, taken in combination with SPACER or SPACER′ or R₂₀ forms a 5- or 6-membered aromatic, heteroaromatic, alicyclic or heteroalicyclic ring, the heteroatoms selected from O, N or S; where Ψ⁻ is a biologically compatible counterion; or
CAP and CAP′ are independently

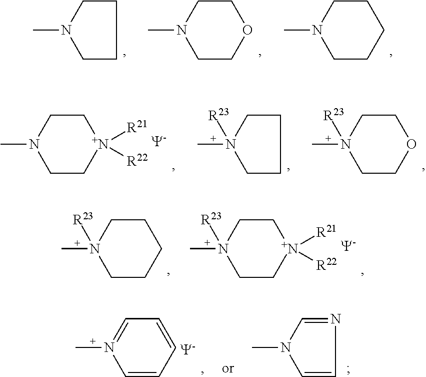

where R₂₁, R₂₂, R₂₃ and Ψ⁻ are as defined previously; such that at least one of R₃, R₄, R₅, R₆, R₇, R₈ or R₁₀ is a TAIL; and, where more than one substituent is a TAIL, each TAIL is optionally the same or different; or
or wherein at least one R₁, R₅-R₁₀ or R₂₀-R₂₃ is a water-soluble group (WSG).

When CAP is —N⁺R₂₁R₂₂R₂₃Ψ⁻, the biologically compatible counter ion balances the positive charge present on the CAP nitrogen, which is a quaternary ammonium salt. As used herein, a substance that is biologically compatible is not toxic as used, and does not have a substantially deleterious effect on biomolecules. Examples of Ψ⁻ include, among others, chloride, bromide, iodide, sulfate, alkanesulfonate, arylsulfonate, phosphate, perchlorate, tetrafluoroboronate, tetraarylboride, nitrate, and anions of aromatic or aliphatic carboxylic acids. Preferred ψ-counterions are bromide, chloride, iodide, perchlorate, and various sulfonates.

The water-soluble group can include a polymer (e.g., a polyalkylene oxide, carbohydrate and polypeptide). In certain embodiments, the water-soluble group comprises a polyethylene oxide. In certain embodiments, R₄ is a TAIL; R₂ is a substituted or unsubstituted phenyl; and/or at least one of R₅, R₆, R₇ and R₈ is —OR₁₁, —SR₁₁, or —NR₉R₁₁). In certain embodiments, n is 0. In certain embodiments, at least one of R₅, R₆, R₇ and R₈ is —OR₁₁. In certain embodiments, —OR₁₁ is —OCH₃. In certain embodiments, R₇ is —OCH₃. In certain embodiments, R₁ is an unsubstituted alkyl and R₁₀ is H. In certain embodiments, R₁ is methyl and R₁₀ is H.

In certain embodiments, wherein if p is 0, and wherein R₁ and R₁₀, which can be the same or different, are independently H; or a substituted or unsubstituted alkyl group, wherein the substitution on the alkyl group, if present, is a carboxyl or (alkyl)ammonium group. In other embodiments, wherein if p is not 0, R₁ and R₁₀, which can be the same or different, are independently a substituted or unsubstituted alkyl group, wherein the substitution on the alkyl group, if present, is a carboxyl or (alkyl)ammonium group.

In certain embodiments, the dye compound of Formula I is selected from the group consisting of Compound 4, Compound 5, Compound 6, Compound 7, Compound 8, Compound 9, Compound 10, Compound 11, Compound 12, Compound 13, Compound 14, Compound 15, Compound 16, Compound 17, Compound 18, Compound 19, Compound 20, Compound 21, Compound 23, Compound 25, Compound 26, Compound 27, Compound 30, Compound 31, Compound 32, Compound 33, Compound 34, Compound 35, Compound 36, Compound 37, Compound 38, Compound 39, Compound 40, Compound 41, Compound 42, Compound 43, Compound 44, Compound 45, Compound 46, Compound 47, Compound 48, Compound 49, Compound 50, Compound 51, Compound 52, Compound 53, Compound 54, Compound 55, Compound 56, Compound 57, Compound 58, Compound 59, Compound 60, Compound 61, Compound 62, Compound 63, Compound 64, and Compound 65.

The dye compounds disclosed herein are typically dissolved or suspended in a carrier for use as a composition. The exact concentration of dye to be used is dependent upon the experimental conditions and the desired results, and optimization of experimental conditions is typically required to determine the best concentration of cyanine dye to be used in a given application. Typically for in vitro use, the concentration of the dye is the minimum amount required to yield a detectable signal in the sample within a reasonable time, with minimal background fluorescence.

Methods of Use:

A general procedure for using the cyanine dyes as disclosed herein for detecting for the presence of RNA molecules in a sample is described below:

A sample suspected of containing RNA (e.g., ssRNA) is combined with a substituted, unsymmetrical cyanine dye compounds provided herein. The sample is incubated for a time sufficient for the dye compound to combine with the RNA in the sample, if present, to form a dye-RNA complex that emits a detectable fluorescent signal. The dye-RNA complex then is detected for the emission of the fluorescent signal, wherein the detectable fluorescent signal is indicative of the presence of a dye-RNA complex.

The sample can be a biological sample, such as cells, cell cultures, tissues, tissue and cell culture medium, organs, serum, whole blood, plasma, bodily fluids, buffer solutions, biological fluids, and the like. The cyanine dye compounds can be formulated with one or more carriers depending on the assay. The dye compounds then are incubated with the sample for a period of time sufficient for the dye compound to associate with the RNA species present in the sample. After such time, the sample is analyzed for fluorescence intensity. The fluorescence intensity after incubation is compared to the fluorescence intensity of the dye in the absence of sample. An increase in the fluorescence intensity of the dye in the biological sample indicates association of the dye with RNA (i.e., the presence of RNA in the sample). The increased fluorescence can be measured or detected using any suitable instrument. Additionally, the fluorescence intensity after incubation can be compared to the fluorescence intensity of the dye in the presence of samples of known RNA concentrations for quantitative measurements.

In one method, a sample suspected of containing ssRNA (e.g., miRNA) is combined with a substituted, unsymmetrical cyanine dye compound provided herein and then incubated for a time sufficient for the dye compound to combine with the ssRNA in the sample, if present, to form a dye-ssRNA complex. Once formed the dye-ssRNA complex can emit a detectable and stable fluorescent signal. After incubation, the emission of the fluorescent signal is detected, where a detectable fluorescent signal is indicative of the presence of a dye-ssRNA complex.

Typically, the fluorescence signal produced by the dye-RNA complex produced by the methods provided herein can be stable for at least 30 minutes at room temperature, and can be stable for much longer, e.g., at least 3 hours at room temperature.

The detection of emitted light from fluorescent RNA-dye complexes can be achieved with any suitable optical detection system that provides spectral information for the complex, e.g., grating spectrometer, prism spectrometer, imaging spectrometer, fluorometer, such as the QUBIT 2.0 Fluorometer (available from Life Technologies Corporation; Carlsbad, Calif.), fluorescence microplate reader, camera, flow cytometers, including bead-based and acoustic focusing flow cytometers, such as the ATTUNE Acoustic Focusing Flow Cytometer (available from Life Technologies Corporation), or the like.

The present methods can be implemented to detect any type of RNA molecule, but are particularly useful in the detection of short RNA molecules. The disclosed methods are well-suited for the detection of short, single-stranded RNA molecules, such as miRNA. Short RNA molecules typically contain a sequence of about 10-100 nucleotides. For example, short RNA molecules can include less than about 50; or less than about 40; or less than about 30 nucleotides; or less than 20 nucleotides. In certain embodiments, the detectable RNA contains 25 or fewer nucleotides. In certain embodiments, methods provided herein can be used to detect RNA formed of a sequence of about 10-20 nucleotides; whereas in other methods, the detectable RNA is formed of a sequence of about 20-25 nucleotides. In certain embodiments, the detectable RNA is single-stranded and contains 25 or fewer nucleotides.

Methods provided herein can be used in the detection of any type of naturally occurring or chemically modified RNA molecule, including but not limited to relatively short ssRNA molecules, such as microRNA (miRNA), small nuclear RNA (snRNA), small nucleolar RNA (snoRNA), tiny non-coding RNA (tncRNA), small modulatory RNA (smRNA), and piwi-interacting RNA (piRNA). In certain embodiments, the RNA molecule can include a chemical modification to at least one sugar, phosphate or base group. For example, one common type of modification utilizes a chemical group other than a hydroxy or phosphate at the 5′ termini of the RNA molecule.

The compositions and methods provided herein can be used to detect the presence of RNA and quantify the amount of RNA in a sample covering a wide range of concentrations. The dye compounds disclosed herein are extremely sensitive to the presence of RNA in a sample. For example, certain assays allow for the detection of RNA (e.g., ssRNA) at concentrations as low as about 25 ng/mL of sample, although higher concentrations of RNA also can be detected using the methods provided herein.

The disclosed assays can be implemented with samples spanning a broad range of ssRNA concentrations. In certain embodiments, the sample can include ssRNA in a concentration of about 2.5 ng/mL to about 1000 ng/mL of sample. In certain embodiments, the sample can include ssRNA in a concentration of about 25 ng/mL to about 1000 ng/mL. In certain embodiments, the sample can include ssRNA in a concentration of about 50 ng/mL to 1000 ng/mL.

In one aspect, a method of detecting the presence of single-stranded RNA (ssRNA) in a sample is provided, the method includes:
(d) combining a sample suspected of containing ssRNA, wherein the ssRNA comprises 25 or fewer nucleotides, with a substituted, unsymmetrical cyanine dye compound;
(e) incubating the sample for a time sufficient for the dye compound to combine with the ssRNA in the sample, if present, to form a dye-ssRNA complex that emits a detectable fluorescent signal; and
(f) detecting the emission of the fluorescent signal, wherein the detectable fluorescent signal is indicative of the presence of a dye-ssRNA complex.

In certain embodiments, the ssRNA comprises 20-25 nucleotides. In certain embodiments, the ssRNA comprises 10-20 nucleotides. In certain embodiments, the ssRNA is naturally occurring. In certain embodiments, the ssRNA comprises a chemical modification to at least one sugar, phosphate or base group. In certain embodiments, the ssRNA comprises a chemical group other than a hydroxyl or phosphate at its 5′ termini. In certain embodiments, the ssRNA is microRNA (miRNA), small nuclear RNA (snRNA), small nucleolar RNA (snoRNA), tiny non-coding RNA (tncRNA), small modulatory RNA (smRNA), or piwi-interacting RNA (piRNA). In certain embodiments, the ssRNA is miRNA.

The methods provided herein can detect as little as 0.5 ng ssRNA in a sample even in the presence of larger RNA, such as mRNA or rRNA. In certain embodiments, the method further comprises detecting ssRNA in a concentration of 0.5 ng/mL or greater. In certain embodiments, the method further comprises detecting ssRNA in a concentration of 5 ng/mL or greater. In certain embodiments, the method further comprises detecting ssRNA in a concentration of 10 ng/mL or greater. In certain embodiments, the method further comprises detecting ssRNA in a concentration of 25 ng/mL or greater. In certain embodiments, the method further comprises detecting ssRNA in a concentration of about 5 ng/mL to about 1000 ng/mL. In certain embodiments, the method further comprises detecting ssRNA in a concentration of about 25 ng/mL to about 1000 ng/mL. In certain embodiments, the method further comprises detecting ssRNA in a concentration of about 50 ng/mL to about 1000 ng/mL. In certain embodiments, the sample can include ssRNA in a concentration of about 5 ng/mL to about 500 ng/mL. In certain embodiments, the sample can include ssRNA in a concentration of about 25 ng/mL to about 500 ng/mL. In certain embodiments, the sample can include ssRNA in a concentration of about 50 ng/mL to about 100 ng/mL.

In certain embodiments, the fluorescence signal is stable for at least 30 minutes at room temperature. In certain embodiments, the fluorescence signal is stable for at least 3 hours at room temperature.

In another aspect, a method of detecting the presence of single-stranded RNA (ssRNA) in a sample is provided, the method includes:
(b) combining a sample suspected of containing ssRNA, wherein the ssRNA comprises 25 or fewer nucleotides, with a substituted, unsymmetrical cyanine dye compound of the Formula I:

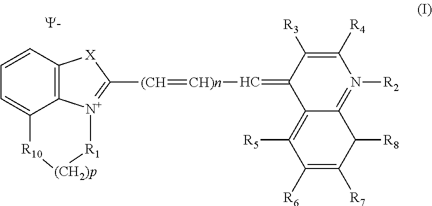

wherein
X is O or S;
n is 0, 1 or 2;
p is 0, 1 or 2;
Ψ is a biologically compatible counterion;
R₁ and R₁₀, which can be the same or different, are independently H; or a substituted or unsubstituted alkyl group, wherein the substitution on the alkyl group, if present, is a carboxyl or (alkyl)ammonium group;
R₂ is a substituted or unsubstituted alkyl, naphthyl, phenyl, thienyl, or cycloalkyl having 3-8 carbons, wherein the substitution on R₂, if present, is a carboxyl or carboxamide group;
R₃ is H; or an alkyl, alkenyl, polyalkenyl, alkynyl or polyalkynyl group; or a halogen; or a substituted or unsubstituted aryl or heteroaryl; or a substituted or unsubstituted cycloalkyl having 3-10 carbons; or —OR₁₁, —SR₁₁, —(NR₉R₁₁); or —OSO₂R₁₉; or a TAIL;
wherein R₉ and R₁₁, which can be the same or different, are independently H; or substituted or unsubstituted alkyl groups; or alkyl groups substituted with one or more alkylammonium groups; or 1-2 alicyclic or aromatic rings; or R₉ and R₁₁ taken in combination are —(CH₂)₄— or —(CH₂)₅— to give a 5 or 6 membered ring; and where R₁₉ is alkyl, or perfluoroalkyl, or aryl;
R₄ is an alkyl, alkenyl, polyalkenyl, alkynyl or polyalkynyl group; or a halogen; or a substituted or unsubstituted aryl or heteroaryl; or a substituted or unsubstituted cycloalkyl having 3-10 carbons; or —OR₁₁, —SR₁₁, —(NR₉R₁₁); or —OSO₂R₁₉; or a TAIL;
R₅, R₆, R₇ and R₈, which can be the same or different, are independently H; or an alkyl, alkenyl, polyalkenyl, alkynyl or polyalkynyl group; or a halogen; or a substituted or unsubstituted aryl or heteroaryl; or a substituted or unsubstituted cycloalkyl having 3-8 carbons; or a TAIL; or —OR₁₁, —SR₁₁, or —(NR₉R₁₁);
TAIL is a heteroatom-containing moiety having the formula LINK-SPACER-CAP;
wherein
LINK is a single covalent bond, —O—, —S—, or —NR₂₀; where R₂₀ is H, a linear or branched alkyl having 1-8 carbons, or R₂₀ is SPACER′-CAP′;
SPACER and SPACER′, which can be the same or different, are covalent linkages, linear or branched, cyclic or heterocyclic, saturated or unsaturated, each having 1-16 nonhydrogen atoms selected from the group consisting of C, N, O and S, such that the linkage begins and ends with a carbon atom, and contains any combination of ether, thioether, amine, ester, amide, or aliphatic, oleic or aromatic carbon-carbon bonds, or aromatic carbon-nitrogen or nitrogen-nitrogen bonds; wherein all heteroatoms in the linear backbone of SPACER are separated by at least two carbon atoms;
CAP and CAP′, which can be the same or different, are —OR₂₁, —SR₂₁, —NR₂₁R₂₂, or —N⁺R₂₁R₂₂R₂₃Ψ⁻;
wherein
R₂₁, R₂₂, and R₂₃ are independently H, or a linear or branched alkyl or cycloalkyl having 1-8 carbons, optionally further substituted by halogen, hydroxy, alkoxy having 1-8 carbons, carboxyalkyl having 1-8 carbons, or phenyl, where phenyl is optionally further substituted by halogen, hydroxy, alkoxy having 1-8 carbons, aminoalkyl having 1-8 carbons, or carboxyalkyl having 1-8 carbons; or, one or more of R₂₁, R₂₂, and R₂₃, taken in combination with SPACER or SPACER′ or R₂₀ forms a 5- or 6-membered aromatic, heteroaromatic, alicyclic or heteroalicyclic ring, the heteroatoms selected from 0, N or S; where Ψ⁻ is a biologically compatible counterion; or
CAP and CAP′ are independently

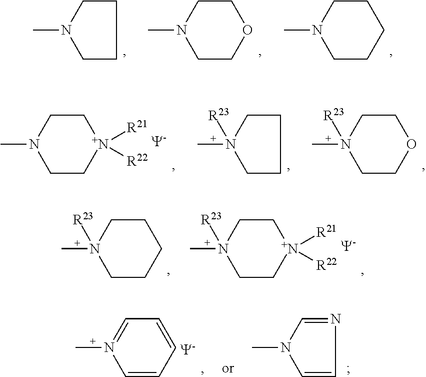

where R₂₁, R₂₂, R₂₃ and Ψ⁻ are as defined previously; such that at least one of R₃, R₄, R₅, R₆, R₇, R₈ or R₁₀ is a TAIL; and, where more than one substituent is a TAIL, each TAIL is optionally the same or different;

or wherein at least one R₁, R₅, R₆, R₇, R₈, R₉, R₁₀, R₁₁ or R₂₀, R₂₁, R₂₂ or R₂₃ is a water-soluble group (WSG);
(d) incubating the sample for a time sufficient for the dye compound to combine with the ssRNA in the sample, if present, to form a dye-ssRNA complex that emits a detectable fluorescent signal; and
(e) detecting the emission of the fluorescent signal, wherein the detectable fluorescent signal is indicative of the presence of a dye-ssRNA complex.

When CAP is —N⁺R₂₁R₂₂R₂₃Ψ⁻, the biologically compatible counter ion Ψ⁻ balances the positive charge present on the CAP nitrogen, which is a quaternary ammonium salt. As used herein, a substance that is biologically compatible is not toxic as used, and does not have a substantially deleterious effect on biomolecules. Examples of Ψ⁻ include, among others, chloride, bromide, iodide, sulfate, alkanesulfonate, arylsulfonate, phosphate, perchlorate, tetrafluoroboronate, tetraarylboride, nitrate, and anions of aromatic or aliphatic carboxylic acids. Preferred ψ-counterions are bromide, chloride, iodide, perchlorate, and various sulfonates.

The water-soluble group can include a polymer (e.g., a polyalkylene oxide, carbohydrate and polypeptide). In certain embodiments, the water-soluble group comprises a polyethylene oxide. In certain embodiments, R₄ is a TAIL; R₂ is a substituted or unsubstituted phenyl; and/or at least one of R₅, R₆, R₇ and R₈ is —OR₁₁, —SR₁₁, or —NR₉R₁₁). In certain embodiments, n is 0. In certain embodiments, at least one of R₅, R₆, R₇ and R₈ is —OR₁₁. In certain embodiments, —OR₁₁ is —OCH₃. In certain embodiments, R₇ is —OCH₃. In certain embodiments, R₁ is an unsubstituted alkyl and R₁₀ is H. In certain embodiments, R₁ is methyl and R₁₀ is H.

In certain embodiments, wherein if p is 0, and wherein R₁ and R₁₀, which can be the same or different, are independently H; or a substituted or unsubstituted alkyl group, wherein the substitution on the alkyl group, if present, is a carboxyl or (alkyl)ammonium group. In other embodiments, wherein if p is not 0, R₁ and R₁₀, which can be the same or different, are independently a substituted or unsubstituted alkyl group, wherein the substitution on the alkyl group, if present, is a carboxyl or (alkyl)ammonium group.

In certain embodiments, the dye compound of Formula I is selected from the group consisting of Compound 4, Compound 5, Compound 6, Compound 7, Compound 8, Compound 9, Compound 10, Compound 11, Compound 12, Compound 13, Compound 14, Compound 15, Compound 16, Compound 17, Compound 18, Compound 19, Compound 20, Compound 21, Compound 23, Compound 25, Compound 26, Compound 27, Compound 30, Compound 31, Compound 32, Compound 33, Compound 34, Compound 35, Compound 36, Compound 37, Compound 38, Compound 39, Compound 40, Compound 41, Compound 42, Compound 43, Compound 44, Compound 45, Compound 46, Compound 47, Compound 48, Compound 49, Compound 50, Compound 51, Compound 52, Compound 53, Compound 54, Compound 55, Compound 56, Compound 57, Compound 58, Compound 59, Compound 60, Compound 61, Compound 62, Compound 63, Compound 64, and Compound 65.

In certain embodiments, the ssRNA comprises 20-25 nucleotides. In certain embodiments, the ssRNA comprises 10-20 nucleotides. In certain embodiments, the ssRNA is naturally occurring. In certain embodiments, the ssRNA comprises a chemical modification to at least one sugar, phosphate or base group. In certain embodiments, the ssRNA comprises a chemical group other than a hydroxyl or phosphate at its 5′ termini. In certain embodiments, the ssRNA is microRNA (miRNA), small nuclear RNA (snRNA), small nucleolar RNA (snoRNA), tiny non-coding RNA (tncRNA), small modulatory RNA (smRNA), or piwi-interacting RNA (piRNA). In certain embodiments, the ssRNA is miRNA.

The methods provided herein can detect as little as 0.5 ng ssRNA in a sample even in the presence of larger RNA, such as mRNA or rRNA. In certain embodiments, the method further comprises detecting ssRNA in a concentration of 0.5 ng/mL or greater. In certain embodiments, the method further comprises detecting ssRNA in a concentration of 5 ng/mL or greater. In certain embodiments, the method further comprises detecting ssRNA in a concentration of 10 ng/mL or greater. In certain embodiments, the method further comprises detecting ssRNA in a concentration of 25 ng/mL or greater. In certain embodiments, the method further comprises detecting ssRNA in a concentration of about 5 ng/mL to about 1000 ng/mL. In certain embodiments, the method further comprises detecting ssRNA in a concentration of about 25 ng/mL to about 1000 ng/mL. In certain embodiments, the method further comprises detecting ssRNA in a concentration of about 50 ng/mL to about 1000 ng/mL. In certain embodiments, the sample can include ssRNA in a concentration of about 5 ng/mL to about 500 ng/mL. In certain embodiments, the sample can include ssRNA in a concentration of about 25 ng/mL to about 500 ng/mL. In certain embodiments, the sample can include ssRNA in a concentration of about 50 ng/mL to about 100 ng/mL.

In certain embodiments, the fluorescence signal is stable for at least 30 minutes at room temperature. In certain embodiments, the fluorescence signal is stable for at least 3 hours at room temperature.

In yet another aspect, a method of detecting single-stranded RNA (ssRNA) in a sample is provided, the method includes:
(d) combining a sample suspected of containing ssRNA, wherein the ssRNA comprises 25 or fewer nucleotides, with a substituted, unsymmetrical cyanine dye compound of the Formula II:

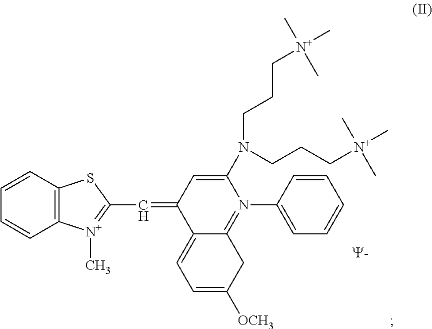

(e) incubating the sample for a time sufficient for the dye compound to combine with the ssRNA in the sample, if present, to form a dye-ssRNA complex that emits a detectable fluorescent signal; and
(f) detecting the emission of the fluorescent signal, wherein the detectable fluorescent signal is indicative of the presence of a dye-ssRNA complex.

In certain embodiments, the ssRNA comprises 20-25 nucleotides. In certain embodiments, the ssRNA comprises 10-20 nucleotides. In certain embodiments, the ssRNA is naturally occurring. In certain embodiments, the ssRNA comprises a chemical modification to at least one sugar, phosphate or base group. In certain embodiments, the ssRNA comprises a chemical group other than a hydroxyl or phosphate at its 5′ termini. In certain embodiments, the ssRNA is microRNA (miRNA), small nuclear RNA (snRNA), small nucleolar RNA (snoRNA), tiny non-coding RNA (tncRNA), small modulatory RNA (smRNA), or piwi-interacting RNA (piRNA). In certain embodiments, the ssRNA is miRNA.

The methods provided herein can detect as little as 0.5 ng ssRNA in a sample even in the presence of larger RNA, such as mRNA or rRNA. In certain embodiments, the method further comprises detecting ssRNA in a concentration of 0.5 ng/mL or greater. In certain embodiments, the method further comprises detecting ssRNA in a concentration of 5 ng/mL or greater. In certain embodiments, the method further comprises detecting ssRNA in a concentration of 10 ng/mL or greater. In certain embodiments, the method further comprises detecting ssRNA in a concentration of 25 ng/mL or greater. In certain embodiments, the method further comprises detecting ssRNA in a concentration of about 5 ng/mL to about 1000 ng/mL. In certain embodiments, the method further comprises detecting ssRNA in a concentration of about 25 ng/mL to about 1000 ng/mL. In certain embodiments, the method further comprises detecting ssRNA in a concentration of about 50 ng/mL to about 1000 ng/mL. In certain embodiments, the sample can include ssRNA in a concentration of about 5 ng/mL to about 500 ng/mL. In certain embodiments, the sample can include ssRNA in a concentration of about 25 ng/mL to about 500 ng/mL. In certain embodiments, the sample can include ssRNA in a concentration of about 50 ng/mL to about 100 ng/mL.

In certain embodiments, the fluorescence signal is stable for at least 30 minutes at room temperature. In certain embodiments, the fluorescence signal is stable for at least 3 hours at room temperature.

In yet another aspect, a method of detecting single-stranded (ss) RNA in a sample is provided, the method includes:
(d) combining a sample suspected of containing ssRNA, wherein the ssRNA comprises 25 or fewer nucleotides, with a substituted, unsymmetrical cyanine dye compound, wherein the dye comprises a first heterocyclic ring system that is a substituted benzothiazolium, benzoxazolium, benzoselenazolium, benzimidazolium, or dialkylindolinium, wherein the first ring system is linked by a monomethine or trimethine bridging moiety attached at the 2-position of the first ring system to the 2- or 4-position of a second heterocyclic ring system that is a substituted quinolinium or that is linked by a monomethine bridging moiety attached to the 2-position of a second heterocyclic ring system that is a substituted pyridinium; wherein one or more substituents of the second ring system is a substituted or unsubstituted cyclohexyl, cyclohexenyl, morpholino, piperidinyl, naphthyl, phenyl, thienyl, benzothizolyl, furanyl, oxazolyl, benzoxazolyl or pyridinyl group;
(e) incubating the sample for a time sufficient for the dye compound to combine with the ssRNA in the sample, if present, to form a dye-ssRNA complex that emits a detectable fluorescent signal; and
(f) detecting the emission of the fluorescent signal, wherein the detectable fluorescent signal is indicative of the presence of a dye-ssRNA complex.

The methods provided herein can be used to detect any type of short RNA molecule. The short RNA molecule can be a single-stranded RNA (ssRNA) molecule. The ssRNA can be naturally occurring or can include a chemical modification to at least one sugar, phosphate or base group. For example, the ssRNA can include a chemical group other than a hydroxy or phosphate at its 5′ terminus. In certain embodiments, the ssRNA molecule can include 20-25 nucleotides. In other embodiments, the ssRNA molecule can include 10-20 nucleotides. In certain methods, the ssRNA is microRNA (miRNA). The ssRNA can be a small nuclear RNA (snRNA), small nucleolar RNA (snoRNA), tiny non-coding RNA (tncRNA), small modulatory RNA (smRNA) or piwi-interacting RNA (piRNA).

The methods provided herein can be used to detect extremely small quantities of RNA (e.g., ssRNA) in a sample. The methods provided herein can detect as little as 0.5 ng ssRNA in a sample even in the presence of larger RNA, such as mRNA or rRNA. In certain embodiments, the method further comprises detecting ssRNA in a concentration of 0.5 ng/mL or greater. In certain embodiments, the method further comprises detecting ssRNA in a concentration of 5 ng/mL or greater. In certain embodiments, the method further comprises detecting ssRNA in a concentration of 10 ng/mL or greater. In certain embodiments, the method further comprises detecting ssRNA in a concentration of 25 ng/mL or greater. In certain embodiments, the method further comprises detecting ssRNA in a concentration of about 5 ng/mL to about 1000 ng/mL. In certain embodiments, the method further comprises detecting ssRNA in a concentration of about 25 ng/mL to about 1000 ng/mL. In certain embodiments, the method further comprises detecting ssRNA in a concentration of about 50 ng/mL to about 1000 ng/mL. In certain embodiments, the sample can include ssRNA in a concentration of about 5 ng/mL to about 500 ng/mL. In certain embodiments, the sample can include ssRNA in a concentration of about 25 ng/mL to about 500 ng/mL. In certain embodiments, the sample can include ssRNA in a concentration of about 50 ng/mL to about 100 ng/mL.

In certain embodiments, the fluorescence signal is stable for at least 30 minutes at room temperature. In certain embodiments, the fluorescence signal is stable for at least 3 hours at room temperature.

Sample Preparation:

The sample may be prepared using methods well known in the art for isolating RNA for in vitro and solution based assay detection or well known methods for live cell or fixed cells for intracellular and/or in vivo detection of RNA. The sample includes, without limitation, any biological derived material that is thought to contain a nucleic acid polymer, such as RNA. Alternatively, samples also include material that nucleic acid polymers, such as RNA, have been added to such as a PCR reaction mixture, a polymer gel such as agarose or polyacrylamide gels or a microfluidic assay system. In another aspect of the present disclosure, the sample can also include a buffer solution that contains RNA to determine the cyanine dye compounds of the present disclosure that are ideal under different assay conditions.

The sample can be a biological fluid such as whole blood, plasma, serum, nasal secretions, sputum, saliva, urine, sweat, transdermal exudates, cerebrospinal fluid, or the like. Biological fluids also include tissue and cell culture medium wherein an analyte of interest has been secreted into the medium. Alternatively, the sample may be whole organs, tissue or cells from the animal. Examples of sources of such samples include muscle, eye, skin, gonads, lymph nodes, heart, brain, lung, liver, kidney, spleen, thymus, pancreas, solid tumors, macrophages, mammary glands, mesothelium, and the like. Cells include without limitation prokaryotic cells such as bacteria, yeast, fungi, mycobacteria and mycoplasma, and eukaryotic cells such as nucleated plant and animal cells that include primary cultures and immortalized cell lines. Typically prokaryotic cells include E. coli and S. aureus. Eukaryotic cells include without limitation ovary cells, epithelial cells, circulating immune cells, β cells, hepatocytes, and neurons.

In an exemplary embodiment, the sample comprises biological fluids, buffer solutions, live cells, fixed cells, eukaryotic cells, prokaryotic cells, nucleic acid polymers, nucleosides, nucleotides, a polymeric gel or tissue sections. In a further aspect, the sample comprises nucleic acid polymers in an aqueous buffer.

The RNA may be either natural (biological in origin) or synthetic (prepared artificially). The presence of the RNA in the sample may be due to a successful or unsuccessful experimental methodology, undesirable contamination, or a disease state. RNA may be present in all, or only part, of a sample, and the presence of RNA may be used to distinguish between individual samples, or to differentiate a portion or region within a single sample.

The RNA may be enclosed in a biological structure, for example contained within a viral particle, an organelle, or within a cell. The RNA enclosed in biological structures may be obtained from a wide variety of environments, including cultured cells, organisms or tissues, unfiltered or separated biological fluids such as urine, cerebrospinal fluid, blood, lymph fluids, tissue homogenate, mucous, saliva, stool, or physiological secretions or environmental samples such as soil, water and air. The RNA may be endogenous or introduced as foreign material, such as by infection or by transfection.

Alternatively, the RNA is not enclosed within a biological structure, but is present as a sample solution. The sample solution can vary from one of purified RNA to crude mixtures such as cell extracts, biological fluids and environmental samples. In some cases it is desirable to separate the RNA from a mixture of biomolecules or fluids in the solution prior to combination with the present cyanine dye compounds. Numerous, well known, techniques exist for separation and purification of RNA from generally crude mixtures with other proteins or other biological molecules.

The sample may be incubated in the presence of the cyanine dye compounds disclosed herein for a time sufficient to form an RNA (e.g., ssRNA)-reporter molecule complex. While permeation and complexation may be more or less rapid for the dye compounds disclosed herein, largely depending on the nature of the substituents present on the dye compound. It should be apparent to one skilled in the art that the time necessary for sufficient permeation of the dye, or sufficient formation of the resulting ssRNA-dye complex, is dependent upon the physical and chemical nature of the individual sample and the sample medium (see for example U.S. Pat. No. 5,658,751).

Illumination:

The sample containing an RNA-dye complex may be illuminated with a wavelength of light selected to give a detectable optical response, and observed with a means for detecting the optical response. By optical response is meant any detectable colorimetric or luminescent property of the complex. Typically, the optical response is related to the excitation or emission properties of the complex.

For example, the sample may be excited by a light source capable of producing light at or near the wavelength of maximum absorption of the fluorescent complex, such as an ultraviolet or visible lamp, an arc lamp, a laser, or even sunlight. The optical response is optionally detected by visual inspection, or by use of any of the following devices: CCD camera, video camera, photographic film, laser-scanning devices, fluorometers, photodiodes, quantum counters, epifluorescence microscopes, scanning microscopes, flow cytometers, fluorescence microplate readers, or by means for amplifying the signal such as photomultiplier tubes. Where the sample is examined using a flow cytometer, examination of the sample optionally includes sorting portions of the sample according to their fluorescence response.

The detection of emitted light from fluorescent RNA-dye complexes can be achieved with any suitable optical detection system that provides spectral information for the complex, e.g., grating spectrometer, prism spectrometer, imaging spectrometer, fluorometer, such as the QUBIT 2.0 Fluorometer (available from Life Technologies Corporation; Carlsbad, Calif.), fluorescence microplate reader, camera, flow cytometers, including bead-based and acoustic focusing flow cytometers, such as the ATTUNE Acoustic Focusing Flow Cytometer (available from Life Technologies Corporation), or the like.

The wavelengths of the excitation and emission bands of the cyanine dye compounds disclosed herein vary with reporter molecule composition to encompass a wide range of illumination and detection bands. This allows the selection of individual reporter molecules for use with a specific excitation source or detection filter. In particular, present reporter molecules can be selected that possess excellent correspondence of their excitation band with the 488 nm band of the commonly used argon laser or emission bands which are coincident with preexisting filters.

The presence, location, and distribution of RNA, particularly single-stranded RNA, may be detected using the spectral properties of the RNA-dye complex. Once the RNA-dye complex is formed, its presence may be detected and used as an indicator of the presence, location, or type of nucleic acids in the sample, or as a basis for sorting cells, or as a key to characterizing the sample or cells in the sample. Such characterization may be enhanced by the use of additional reagents, including fluorescent reagents. The RNA (e.g., ssRNA) concentration in a sample can also be quantified by comparison with known relationships between the fluorescence of the RNA-dye complex and concentration of nucleic acids in the sample. In particular, fluorescence intensity may be compared to a standard curve prepared from samples containing known nucleic acid concentrations, particularly RNA concentrations.

Kits:

The substituted unsymmetrical cyanine dye compounds for use in the disclosed methods and compositions described herein also can form part of a kit. Exemplary kits provided herein can include one or more substituted unsymmetrical cyanine dye compound, as disclosed herein (e.g., a dye of Formula I or II) and instructions for using the dye in a method for detecting for the presence of ssRNA in a sample.

In yet another aspect, a kit for detecting the presence of single-stranded RNA (ssRNA) in a sample is provided that includes:

a dye of the Formula II; and

instructions for using the dye in a method as disclosed herein.

In yet another aspect, a kit for detecting the presence of single-stranded RNA (ssRNA) in a sample is provided that includes:

a dye of the Formula I:

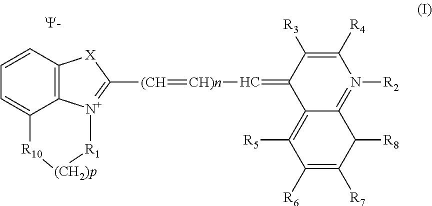

wherein
X is O or S;
n is 0, 1 or 2;
p is 0, 1 or 2;
Ψ is a biologically compatible counterion;
R₁ and R₁₀, which can be the same or different, are independently H; or a substituted or unsubstituted alkyl group, wherein the substitution on the alkyl group, if present, is a carboxyl or (alkyl)ammonium group;
R₂ is a substituted or unsubstituted alkyl, naphthyl, phenyl, thienyl, or cycloalkyl having 3-8 carbons, wherein the substitution on R₂, if present, is a carboxyl or carboxamide group;
R₃ is H; or an alkyl, alkenyl, polyalkenyl, alkynyl or polyalkynyl group; or a halogen; or a substituted or unsubstituted aryl or heteroaryl; or a substituted or unsubstituted cycloalkyl having 3-10 carbons; or —OR₁₁, —SR₁₁, —(NR₉R₁₁); or —OSO₂R₁₉; or a TAIL;
wherein R₉ and R₁₁, which can be the same or different, are independently H; or substituted or unsubstituted alkyl groups; or alkyl groups substituted with one or more alkylammonium groups; or 1-2 alicyclic or aromatic rings; or R₉ and R₁₁ taken in combination are —(CH₂)₄— or —(CH₂)₅— to give a 5 or 6 membered ring; and where R₁₉ is alkyl, or perfluoroalkyl, or aryl;
R₄ is an alkyl, alkenyl, polyalkenyl, alkynyl or polyalkynyl group; or a halogen; or a substituted or unsubstituted aryl or heteroaryl; or a substituted or unsubstituted cycloalkyl having 3-10 carbons; or —OR₁₁, —SR₁₁, —(NR₉R₁₁); or —OSO₂R₁₉; or a TAIL;
R₅, R₆, R₇ and R₈, which can be the same or different, are independently H; or an alkyl, alkenyl, polyalkenyl, alkynyl or polyalkynyl group; or a halogen; or a substituted or unsubstituted aryl or heteroaryl; or a substituted or unsubstituted cycloalkyl having 3-8 carbons; or a TAIL; or —OR₁₁, —SR₁₁, or —(NR₉R₁₁);
TAIL is a heteroatom-containing moiety having the formula LINK-SPACER-CAP;
wherein
LINK is a single covalent bond, —O—, —S—, or —NR₂₀; where R₂₀ is H, a linear or branched alkyl having 1-8 carbons, or R₂₀ is SPACER′-CAP′;
SPACER and SPACER′, which can be the same or different, are covalent linkages, linear or branched, cyclic or heterocyclic, saturated or unsaturated, each having 1-16 nonhydrogen atoms selected from the group consisting of C, N, O and S, such that the linkage begins and ends with a carbon atom, and contains any combination of ether, thioether, amine, ester, amide, or aliphatic, oleic or aromatic carbon-carbon bonds, or aromatic carbon-nitrogen or nitrogen-nitrogen bonds; wherein all heteroatoms in the linear backbone of SPACER are separated by at least two carbon atoms;
CAP and CAP′, which can be the same or different, are —OR₂₁, —SR₂₁, —NR₂₁R₂₂, or —N⁺R₂₁R₂₂R₂₃Ψ⁻;
wherein
R₂₁, R₂₂, and R₂₃ are independently H, or a linear or branched alkyl or cycloalkyl having 1-8 carbons, optionally further substituted by halogen, hydroxy, alkoxy having 1-8 carbons, carboxyalkyl having 1-8 carbons, or phenyl, where phenyl is optionally further substituted by halogen, hydroxy, alkoxy having 1-8 carbons, aminoalkyl having 1-8 carbons, or carboxyalkyl having 1-8 carbons; or, one or more of R₂₁, R₂₂, and R₂₃, taken in combination with SPACER or SPACER′ or R₂₀ forms a 5- or 6-membered aromatic, heteroaromatic, alicyclic or heteroalicyclic ring, the heteroatoms selected from O, N or S; where Ψ⁻ is a biologically compatible counterion; or
CAP and CAP′ are independently

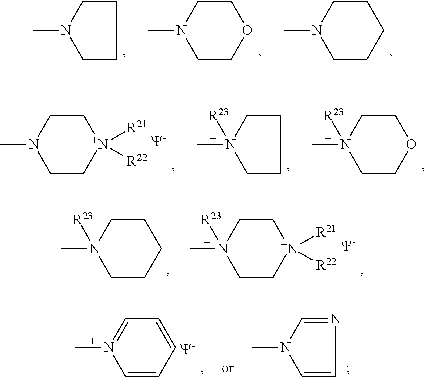

where R₂₁, R₂₂, R²³ and Ψ⁻ are as defined previously; such that at least one of R₃, R₄, R₅, R₆, R₇, R₈ or R₁₀ is a TAIL; and, where more than one substituent is a TAIL, each TAIL is optionally the same or different; or
or wherein at least one of R₁, R₅, R₆, R₇, R₈, R₉, R₁₀, R₁₁ or R₂₀, R₂₁, R₂₂ or R₂₃ is a water-soluble group (WSG); and

instructions for using the dye in a method as disclosed herein.

When CAP is —N⁺R₂₁R₂₂R₂₃Ψ⁻, the biologically compatible counter ion balances the positive charge present on the CAP nitrogen, which is a quaternary ammonium salt. As used herein, a substance that is biologically compatible is not toxic as used, and does not have a substantially deleterious effect on biomolecules. Examples of Ψ⁻ include, among others, chloride, bromide, iodide, sulfate, alkanesulfonate, arylsulfonate, phosphate, perchlorate, tetrafluoroboronate, tetraarylboride, nitrate, and anions of aromatic or aliphatic carboxylic acids. Preferred ψ-counterions are bromide, chloride, iodide, perchlorate, and various sulfonates.

The water-soluble group can include a polymer (e.g., a polyalkylene oxide, carbohydrate and polypeptide). In certain embodiments, the water-soluble group comprises a polyethylene oxide. In certain embodiments, R₄ is a TAIL; R₂ is a substituted or unsubstituted phenyl; and/or at least one of R₅, R₆, R₇ and R₈ is —OR₁₁, —SR₁₁, or —NR₁₁R₉). In certain embodiments, n is 0. In certain embodiments, at least one of R₅, R₆, R₇ and R₈ is —OR₁₁. In certain embodiments, —OR₁₁ is —OCH₃. In certain embodiments, R₇ is —OCH₃. In certain embodiments, R₁ is an unsubstituted alkyl and R₁₀ is H. In certain embodiments, R₁ is methyl and R₁₀ is H.

In certain embodiments, wherein if p is 0, and wherein R₁ and R₁₀, which can be the same or different, are independently H; or a substituted or unsubstituted alkyl group, wherein the substitution on the alkyl group, if present, is a carboxyl or (alkyl)ammonium group. In other embodiments, wherein if p is not 0, R₁ and R₁₀, which can be the same or different, are independently a substituted or unsubstituted alkyl group, wherein the substitution on the alkyl group, if present, is a carboxyl or (alkyl)ammonium group.

In certain embodiments, the dye compound of Formula I is selected from the group consisting of Compound 4, Compound 5, Compound 6, Compound 7, Compound 8, Compound 9, Compound 10, Compound 11, Compound 12, Compound 13, Compound 14, Compound 15, Compound 16, Compound 17, Compound 18, Compound 19, Compound 20, Compound 21, Compound 23, Compound 25, Compound 26, Compound 27, Compound 30, Compound 31, Compound 32, Compound 33, Compound 34, Compound 35, Compound 36, Compound 37, Compound 38, Compound 39, Compound 40, Compound 41, Compound 42, Compound 43, Compound 44, Compound 45, Compound 46, Compound 47, Compound 48, Compound 49, Compound 50, Compound 51, Compound 52, Compound 53, Compound 54, Compound 55, Compound 56, Compound 57, Compound 58, Compound 59, Compound 60, Compound 61, Compound 62, Compound 63, Compound 64, and Compound 65.

In yet another aspect, a kit for detecting the presence of single-stranded RNA (ssRNA) in a sample is provided that includes:

a dye compound selected from the group consisting of Compound 4, Compound 5, Compound 6, Compound 7, Compound 8, Compound 9, Compound 10, Compound 11, Compound 12, Compound 13, Compound 14, Compound 15, Compound 16, Compound 17, Compound 18, Compound 19, Compound 20, Compound 21, Compound 23, Compound 25, Compound 26, Compound 27, Compound 30, Compound 31, Compound 32, Compound 33, Compound 34, Compound 35, Compound 36, Compound 37, Compound 38, Compound 39, Compound 40, Compound 41, Compound 42, Compound 43, Compound 44, Compound 45, Compound 46, Compound 47, Compound 48, Compound 49, Compound 50, Compound 51, Compound 52, Compound 53, Compound 54, Compound 55, Compound 56, Compound 57, Compound 58, Compound 59, Compound 60, Compound 61, Compound 62, Compound 63, Compound 64, and Compound 65; and

instructions for using the dye in a method as disclosed herein.

In yet another aspect, a kit for detecting the presence of single-stranded RNA (ssRNA) in a sample is provided that includes:

a substituted, unsymmetrical cyanine dye compound, wherein the dye comprises a first heterocyclic ring system that is a substituted benzothiazolium, benzoxazolium, benzoselenazolium, benzimidazolium, or dialkylindolinium, wherein the first ring system is linked by a monomethine or trimethine bridging moiety attached at the 2-position of the first ring system to the 2- or 4-position of a second heterocyclic ring system that is a substituted quinolinium or that is linked by a monomethine bridging moiety attached to the 2-position of a second heterocyclic ring system that is a substituted pyridinium; wherein one or more substituents of the second ring system is a substituted or unsubstituted cyclohexyl, cyclohexenyl, morpholino, piperidinyl, naphthyl, phenyl, thienyl, benzothizolyl, furanyl, oxazolyl, benzoxazolyl or pyridinyl group; and

instructions for using the dye in a method as disclosed herein.

Such kits can be prepared from readily available materials and reagents and can come in a variety of embodiments. The contents of the kit will depend on the design of the assay protocol or reagent for detection or measurement. All kits will contain instructions, appropriate reagents, and one or more of the presently disclosed cyanine dye compounds. Typically, instructions include a tangible expression describing the reagent concentration or at least one assay method parameter such as the relative amounts of reagent and sample to be added together, maintenance time periods for reagent/sample admixtures, temperature, buffer conditions and the like to allow the user to carry out any one of the methods or preparations described above. In one aspect, the kit is formulated to facilitate the high-throughput screening of multiple samples, such as may be accomplished using automated methods.

Thus, a kit for detecting nucleic acid in a sample may comprise a dye compound as described above. The kit may further include instructions for performing one or more of the above disclosed methods, including the detection and/or quantitation of ssRNA.

The kit may optionally further include one or more of the following; sample preparation reagents, a buffering agent, nucleic acid standards, an aqueous nucleic acid reporter molecule dilution buffer, an organic solvent or an additional detection reagent, particularly where the additional detection reagent is an additional distinct nucleic acid reporter molecule.

In an exemplified embodiment, the dilution buffer (for the nucleic acid reporter molecule) contains a detergent in a final concentration of about 0.01% to about 0.5% (w/v). The detergents are as disclosed about and include CHAPS, TRITON-X, SDS and TWEEN 20.

The foregoing kits having been described it is understood that the many and varied compounds disclosed herein can be utilized with the many kits. The compounds not being limited to just those that are specifically disclosed.

The following examples are offered to illustrate but not to limit the embodiments described herein.

### EXAMPLES

### Example 1

### MyQubit microRNA Assay

The MyQubit miRNA Assay can be performed using a QUBIT 2.0 Fluorometer (available from Life Technologies Corporation; Cat. no. Q32866) equipped with V3.10 firmware or later to measure the concentration of microRNA in a sample.

The assay utilizes the dye compound of Formula II (referred to in this example as the “binding dye”), which exhibits a large increase in fluorescence upon binding to single-stranded DNA molecules. The assay described herein uses a chemically modified siRNA 21-mer (SILENCER Select GAPDH from Life Technologies Corporation).

The binding dye has been used to reproducibly quantify siRNA in pure samples at levels as low as 0.5 ng in the assay tube following the below protocol. The assay also can be applied to quantify miRNA in samples to similarly low levels.

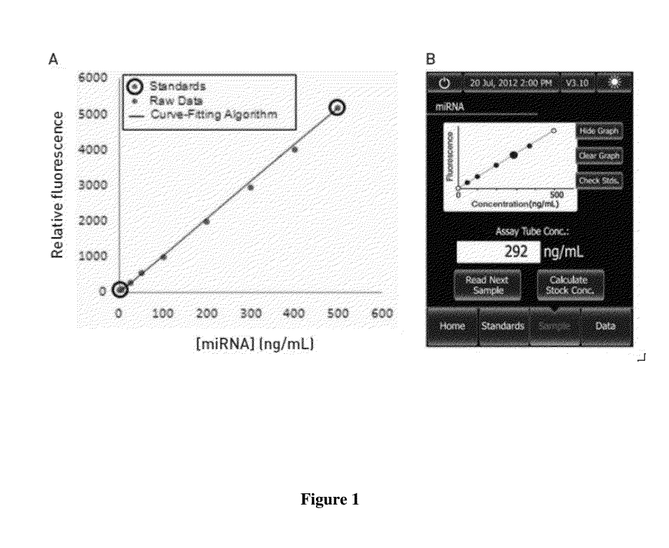

The MyQubit miRNA Assay has a core dynamic range of 5-500 ng/mL miRNA in the assay tube (FIG. 1). Because the assay tolerates 1-20 μL of sample in an assay volume of 200 μL, accurate results can be obtained for initial sample concentrations from about 50 ng/mL-100 ng/mL.

The following materials from Life Technologies Corporation are used in the assay: 20×TE Buffer (pH 7.5) (10 mM Tris, 1 mM EDTA (pH 7.5)); miRNA Std. 1 (Cat. no. T11493); 20×TE Buffer (pH 7.5) +0.01% CHAPS (w/v) (e.g., ZOOM CHAPS, Cat. no. ZC10003); SILENCER Select GAPDH Positive Control siRNA (Hs, Mm, Rn); and miRNA Std. 2 (Cat. no. 4390849); QUBIT Assay Tubes (Cat. no. Q32856) or AXYGEN PCR-05-C tubes (VWR part no. 10011-830); and plastic tubes for preparing buffers and dilutions of standards/samples. The amount of binding dye indicated above provides enough material for 1000 miRNA assays using the QUBIT 2.0 Fluorometer and following the protocol described below.

Material Storage and Handling:

The binding dye should be stored at room temperature and protected from light. Buffers can be stored at room temperature for short periods of time (weeks); for long-term storage, these components should be stored at 4° C. SILENCER Select siRNA should be stored at <−20° C. until use. Once reconstituted, the siRNA (and subsequent dilutions) should be stored at 4° C. The product information provided with the materials provides additional storage and handling information, including disposal.

Instrument Set-Up:

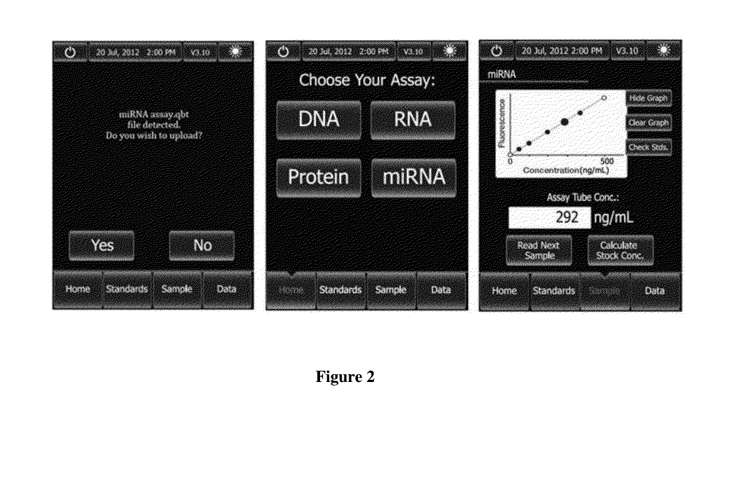

The MyQubit miRNA Assay file (available at www.lifetechnologies.com/qubit) is uploaded to the fluorometer following the manufacturer's instruction (FIG. 2). The QUBIT 2.0 Fluorometer will recognize the miRNA assay.qbt file and guide the user through the upload process.

The assay can be run using the following parameters:

Assay Temperature:

The MyQubit miRNA Assay is designed to be performed at room temperature and delivers optimal performance when all solutions are at room temperature (22-28° C.). Temperature fluctuations can influence the accuracy of the assay. To minimize temperature fluctuations, store the binding dye and assay buffer at room temperature and insert all assay tubes into the fluorometer only for as much time as it takes for the instrument to measure the fluorescence; the fluorometer can raise the temperature of the assay solution, even over a period of a few minutes. The assay tubes should not be held in the hand before reading, as this warms the solution and can result in a low reading.

Incubation Time:

To allow the MyQubit miRNA Assay to reach optimal fluorescence, incubate the tubes for 2 minutes after mixing the sample or standard with the working solution. After this incubation period, the fluorescence signal is stable for at least 3 hours at room temperature.

Calibrating the QUBIT 2.0 Fluorometer:

For each assay, one has the choice to run a new calibration or to use the values from the previous calibration. When first using the instrument for the described assay, it is recommended to conduct a new calibration be run each time. Once familiar with the assays, the instrument, pipetting accuracy, and temperature fluctuations within the laboratory, it can be acceptable to use the calibration data stored from the last time the assay was calibrated. The fluorescence signal in the tubes containing standards and the samples is stable for at least 3 hours when stored at room temperature. Therefore, it is recommended that a new calibration be performed each time a new working solution is prepared.

Calculating the Sample Concentration:

The QUBIT 2.0 Fluorometer gives values for the MyQubit miRNA Assay in ng/mL. This value corresponds to the concentration after the sample is diluted into the assay tube. The following equation can be used to calculate the concentration of the starting sample:

Concentration of sample=QF value×(200/x),

where QF value is the value given by the fluorometer, and x is the volume of sample in microliters added to the assay tube.

Dilution Calculator:

The Dilution Calculator feature of the QUBIT 2.0 Fluorometer calculates the concentration of the original sample based on the volume of sample in the assay tube. To have the fluorometer perform this calculation, press Calculate Stock Concentration after a sample measurement is completed. Using the volume scroll wheel, select the volume of the original sample that is added to the assay tube. The fluorometer calculates the original sample concentration based on the measured assay concentration.

To save the data from the calculation to the fluorometer, press Save on the Dilution Calculator screen. The last calculated value of the measurement is saved to the instrument with a date and time stamp.

The Dilution Calculator generates a concentration for the original sample with the same units as the value in the assay tube given by the fluorometer. The units displayed in this calculation cannot be changed on the instrument for assays accessed using MyQubit.

Experimental Procedure:

Buffers and reagents are prepared as follows:
2.1 Allow all reagents to come to room temperature.
2.2 The binding dye comes as a 200× concentrate in DMSO. The stock solution is ready-to-use. No intermediate dilutions are required.
2.3 The buffer used to prepare standard dilutions is 1× TE Buffer (10 mM Tris, 1 mM EDTA, pH 7.5), which can be prepared by diluting 20×TE Buffer (Cat. no. T11493) in E-pure water. This buffer solution also acts as Standard #1 in the calibration of the miRNA assay. 1 mL solution of 1× TE Buffer provides enough material for 100 assay calibrations.
2.4 Prepare 1× TE Buffer+0.01% CHAPS (w/v), which serves as the assay buffer. All dye working solutions should be prepared in TE Buffer+0.01% CHAPS (w/v). For convenience, one can prepare an aqueous stock solution of 1% CHAPS (w/v) and dilute to 0.01% (w/v) accordingly. A solution of 200 mL buffer (20 ng of CHAPS in 200 mL of 1× TE buffer) is sufficient for 1000 assays, following the standard MyQubit miRNA Assay protocol described below. In the presence of CHAPS, dye working solution is stable for at least 3 hours at room temperature.
2.5 Prepare a 100 ng/μL SILENCER Select siRNA solution by adding 650 μL of 1×TE Buffer only (no CHAPS) to the SILENCER Select siRNA (5 nmol, or 65 μg).
2.6 Prepare a 10 ng/μL SILENCER Select siRNA stock solution by diluting the 100 ng/μL siRNA solution 10-fold in 1× TE Buffer only (no CHAPS) (add 100 μL of 100 ng/μL siRNA solution to 900 μL of 1× TE Buffer). This solution is Standard #2 in the calibration of the QUBIT miRNA Assay. 1 mL of the siRNA stock solution provides enough material for 100 assay calibrations.

Performing the MyQubit miRNA Assay

The protocol below describes the MyQubit miRNA Assay in a total volume of 200 μL per assay tube (see Table 2). The final concentrations of assay standards and samples are adjusted for the additional volume based on the dilution scheme outlined in the protocol below.

3.1 Set up two assay tubes for the standards and one for each user sample. Use only thin-walled, clear 0.5 mL PCR tubes. Acceptable tubes include QUBIT assay tubes (set of 500, Cat. no. Q32856) or AXYGEN PCR-05-C tubes (VWR, part no. 10011-830).
3.2 Prepare 200 μL of dye working solution for each standard and sample by diluting the ssDNA reagent 1:200 in 1× TE Buffer+0.01% CHAPS (w/v). For example, if assaying 5 samples, prepare 1 mL of dye working solution for the samples and 400 μL for the two standards, for a total of 1.4 mL of dye working solution.
3.3 Prepare calibration standards by combining 10 μL of Standard #1 (1×TE Buffer only) with 190 μL of dye working solution, and 10 μL Standard #2 (10 ng/μL of SILENCER Select siRNA, from Step 2.6 above) with 190 μL of dye working solution.
3.4 Prepare samples by combining 1-20 μL of sample with 180-199 μL of dye working solution.

TABLE 2
Volume of reagents used in the assay

| | Standard | User Sample | |
|---|---|---|---|
| | Assay Tube | Assay Tube | |
| Volume of Dye Working Solution | 190 μL | 180-199 | μL |
| (from Step 3.2) | | | |
| Volume of Standard | 10 μL | — | |
| Volume of User Sample | — | 1-20 | μL |
| Total volume in each Assay Tube | 200 μL | 200 | μL |

3.5 Vortex all of the tubes for 2-3 seconds and incubate at room temperature for 2 minutes.
3.6 Calibrate the assay and read samples by selecting “miRNA” from the Home Screen and following the on-screen instructions.
3.7 Optional: Using the Dilution Calculator function of the QUBIT 2.0 Fluorometer, determine the stock concentration of the original sample.

### Example 2

### miRNA and RNA Assays—Selectivity

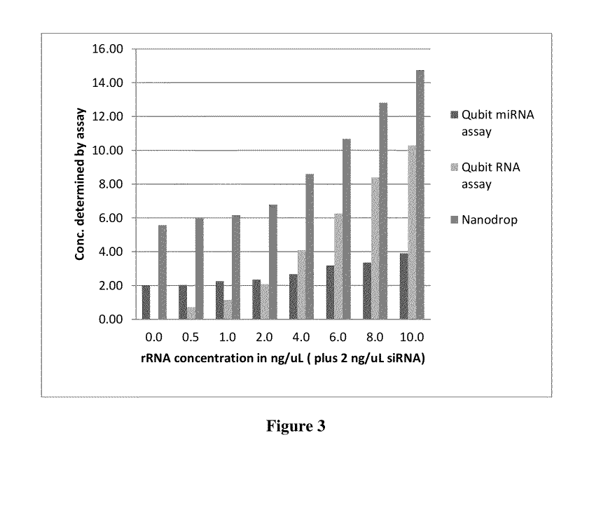

rRNA (Life Technologies cat. #Q32852 component D, diluted in 10 mM Tris pH 7.5, 1 mM EDTA) at the concentrations listed in FIG. 3, was spiked into 2 ng/μL siRNA (Life Technologies cat. #4390849, diluted in 10 mM Tris pH 7.5, 1 mM EDTA) and then read in the MyQubit miRNA and QUBIT RNA assays, as described in Example 1 (Life Technologies Corporation), as well as on the NANODROP Spectrophotometer (ThermoScientific, Waltham, Mass.) at an absorbance of 260 nm. Optical density values from the NANODROP instrument were converted to ng/μL using the following conversion factors: ssRNA: 1 A260=40 ng/μL; dsRNA: 1 A260=45 ng/μL. Eight replicates were used for all three methods.

The MyQubit miRNA assay read 102-196% of the siRNA even in the presence of 5-fold (500%) more rRNA than siRNA. The QUBIT RNA assay read 103-146% of the rRNA concentrations, even in the presence of 4-fold (400%) more siRNA than rRNA. The siRNA and rRNA concentrations read in the NANODROP Spectrophotometer were at the low-end of its detection limit. (see FIG. 3).

### Example 3

### miRNA Assays Detection of Small ssRNA and dsRNA

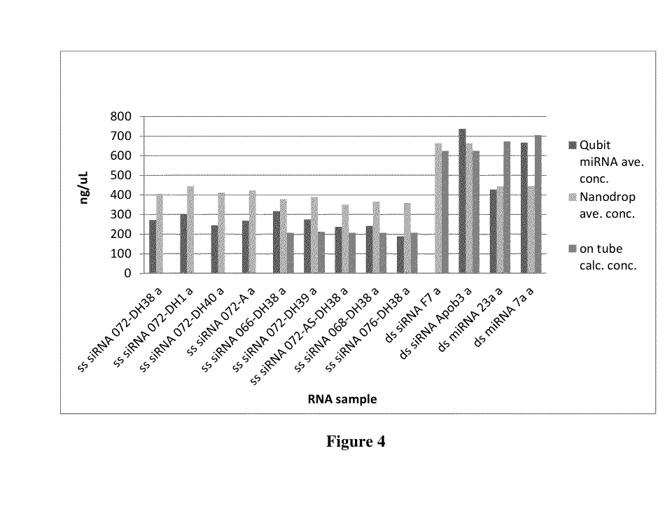

Single-stranded RNA's (ssRNA) were read at their initial concentration on the NANODROP Spectrophotometer and then diluted 1:50 for the MyQubit miRNA Assay. Double-stranded RNA's (dsRNA) were diluted 1:10, read on the NANODROP Spectrophotometer and then were further diluted 1:50 for the MyQubit miRNA Assay. The MyQubit miRNA assays were carried out as described in Example 1. The conversion formulae used for converting the absorbance at 260 nm to nucleic acid concentration using the NANODROP Spectrophotometer were as follows: ssRNA: 1 A₂₆₀=40 ng/μL; dsRNA: 1 A₂₆₀=45 ng/μL. All miRNA information can be found at www.mirbase.org. The miRNAs used in this assay were hsa-let-7a which has a length of 22 nucleotides, and hsa-miR-23a, which has a length of 21 nucleotides. The estimated concentrations of the ssRNA and dsRNA are shown in FIG. 4, comparing the concentrations derived from the MyQubit miRNA Assay (black bars), the NANODROP Spectrophotometer (light gray bars), and the value that was written on each tube (dark gray bars). Tubes of RNA were obtained from Sasha Vlassov at AMBION (division of Life Technologies) in Austin, Tex.

### Example 4

### Concentration of RNA Extracted from U2OS Cells Using the MyQubit miRNA Assay and QUBIT RNA Assay

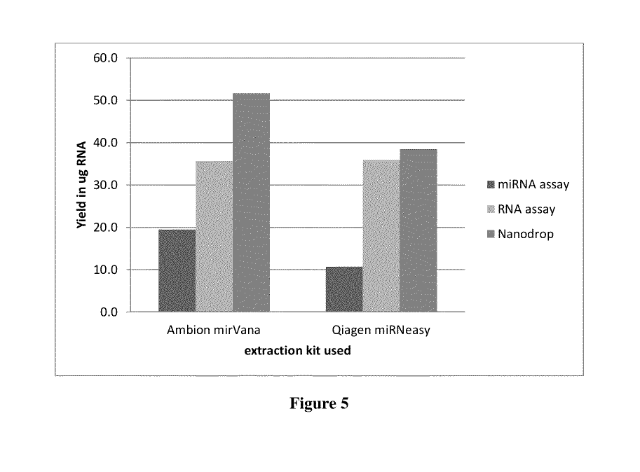

RNA was extracted from three different aliquots of U2OS cells, 2,400,000 cells in each aliquot, and processed according to the manufacturer's protocols in the AMBION MIRVANA kit (Life Technologies Corp.), and the QIAGEN MIRNEASY kit (QIAGEN, Netherlands). Stock concentrations were determined in triplicate readings on the NANODROP Spectrophotometer at 260 nm, using 1 A₂₆₀=40 ng/μL. Stock RNAs were diluted 1:100 and the concentrations were determined in triplicate in the MyQubit miRNA and QUBIT RNA Assays. FIG. 5 shows the yield of RNA as determined by the MyQubit miRNA Assay (black bars), QUBIT RNA Assay (light gray bars), and the NANODROP Spectrophotometer (dark gray bars).

The concentrations determined by the MyQubit miRNA Assay were about 1/3 of the concentrations determined by the NANODROP Spectrophotometer, suggesting that the MyQubit miRNA Assay is less specific for larger RNA, such as rRNA (see FIG. 3).

### Example 5

### Detection of mRNA in the MyQubit miRNA Assay

Two mRNAs (EGFP mRNA, TriLink Biotechnologies cat. #L-6101, 996 nt long, and Flue mRNA, TriLink Biotechnologies cat. #L-6107, 1929 nt long) were tested in the MyQubit miRNA and QUBIT RNA Assays. The mRNAs were diluted in 1× TE buffer, pH 7.5 to a final concentration of 15 ng/μL, and the concentrations were measured by absorbance at 258 nm on a NANODROP Spectrophotometer using 1 A₂₅₈=40 ng/μL. The 15 ng/μL solutions were further diluted to 0.1, 0.5, 2, 4, 8 and 10 ng/μL. For each assay, 50 μL of each dilution was aliquotted and 950 μL of working solution was added. 200 μL of each of the resulting samples was aliquotted to each of five MyQubit assay tubes and read in the respective assay (5 replicates).

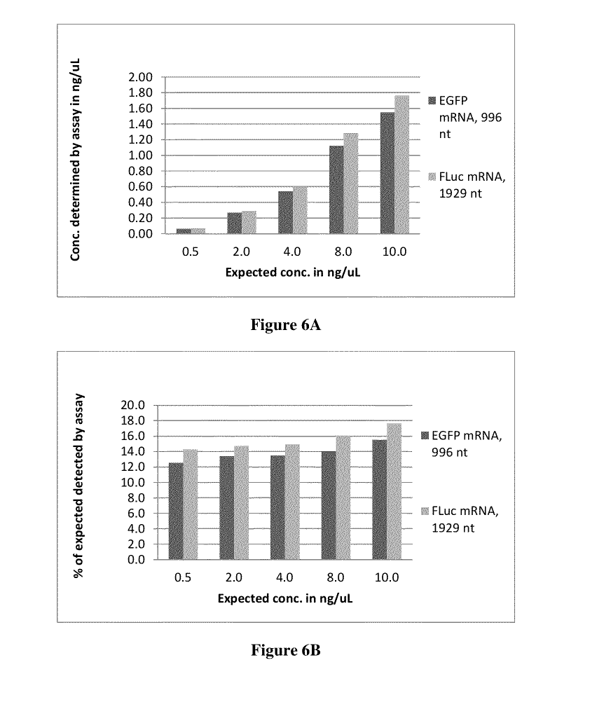

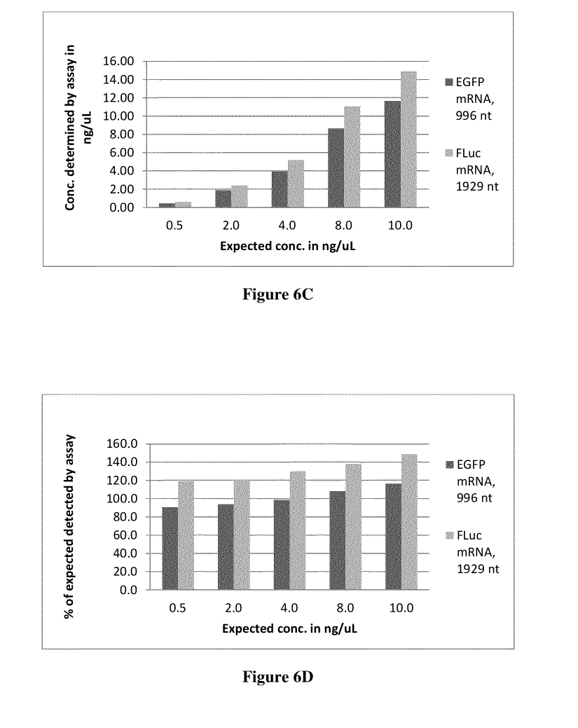

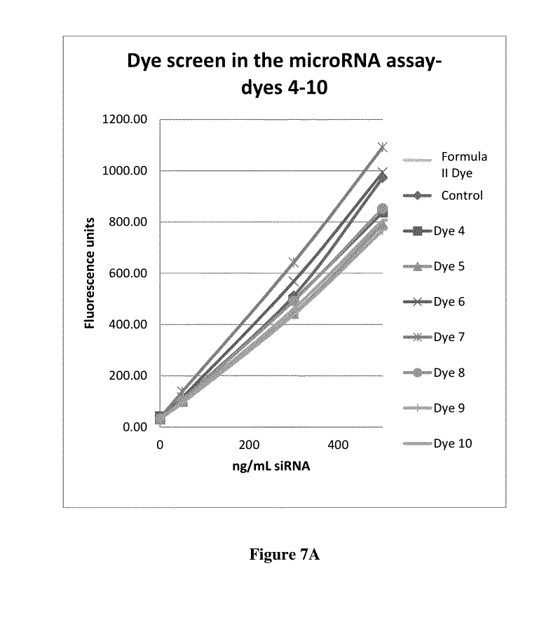

The results of each assay are shown as follows: FIGS. 6A and 6B: The MyQubit miRNA Assay detected about 15% of each mRNA; FIGS. 6C and 6D: The QUBIT RNA Assay detected 90-150% of each mRNA.

### Example 6

### Detection of Nucleotides in the MyQubit miRNA Assay

Pure single NTPs (ATP, CTP, GTP and UTP) were diluted 1× in TE buffer, pH 7.5 to a final concentration of 10 ng/μL. The concentration of each dilution was confirmed by A₂₆₀. 10 μL of each diluted NTP was aliquotted into QUBIT Assay tubes in triplicate for analysis in MyQubit miRNA and QUBIT RNA Assays. The four nucleotide solutions were also mixed in equal proportions and 10 μL of the NTP mixture was aliquotted in triplicate into QUBIT Assay tubes for analysis in MyQubit miRNA and QUBIT RNA Assays. 190 μL of miRNA or RNA Assay Working Solution was added to each sample and then read in the respective QUBIT Assay.

The signal for all of the single NTP and mixed NTP samples was very close to background, indicating that nucleotides were not detected by either assay (see Tables 3 and 4).

TABLE 3
MyQubit miRNA Assay

| | Expected conc. | Assay | |
|---|---|---|---|
| Name | (in assay tube) | Conc. | Units |
| std 2 | 500 ng/mL | 500 | ng/mL |
| std 2* | 500 ng/mL | 497 | ng/mL |
| std 1 | 0 | <2.5 | ng/mL |
| std 1* | 0 | <2.5 | ng/mL |
| mixed | 500 ng/mL | <2.5 | ng/mL |
| NT's | | | |
| mixed | 500 ng/mL | <2.5 | ng/mL |
| NT's | | | |
| mixed | 500 ng/mL | <2.5 | ng/mL |
| NT's | | | |
| UTP | 500 ng/mL | <2.5 | ng/mL |
| UTP | 500 ng/mL | <2.5 | ng/mL |
| UTP | 500 ng/mL | <2.5 | ng/mL |
| GTP | 500 ng/mL | <2.5 | ng/mL |
| GTP | 500 ng/mL | <2.5 | ng/mL |
| GTP | 500 ng/mL | <2.5 | ng/mL |
| CTP | 500 ng/mL | <2.5 | ng/mL |
| CTP | 500 ng/mL | <2.5 | ng/mL |
| CTP | 500 ng/mL | <2.5 | ng/mL |
| ATP | 500 ng/mL | <2.5 | ng/mL |
| ATP | 500 ng/mL | <2.5 | ng/mL |
| ATP | 500 ng/mL | <2.5 | ng/mL |

TABLE 4
QUBIT RNA Assay

| | Expected conc. | Assay | |
|---|---|---|---|
| Name | (in assay tube) | Conc. | Units |
| std 2 | 500 ng/mL | 500 | ng/ml |
| std 2* | 500 ng/mL | 494 | ng/ml |
| std 1 | 0 | <20 | ng/ml |
| std 1* | 0 | <20 | ng/ml |
| mixed | 500 ng/mL | <20 | ng/ml |
| NT's | | | |
| mixed | 500 ng/mL | <20 | ng/ml |
| NT's | | | |
| mixed | 500 ng/mL | <20 | ng/ml |
| NT's | | | |
| UTP | 500 ng/mL | <20 | ng/ml |
| UTP | 500 ng/mL | <20 | ng/ml |
| UTP | 500 ng/mL | <20 | ng/ml |
| GTP | 500 ng/mL | <20 | ng/ml |
| GTP | 500 ng/mL | <20 | ng/ml |
| GTP | 500 ng/mL | <20 | ng/ml |
| CTP | 500 ng/mL | <20 | ng/ml |
| CTP | 500 ng/mL | <20 | ng/ml |
| CTP | 500 ng/mL | <20 | ng/ml |
| ATP | 500 ng/mL | <20 | ng/ml |
| ATP | 500 ng/mL | <20 | ng/ml |
| ATP | 500 ng/mL | <20 | ng/ml |

### Example 7

### Analysis of Dye Compounds of the Present Teachings in the MyQubit miRNA Assay

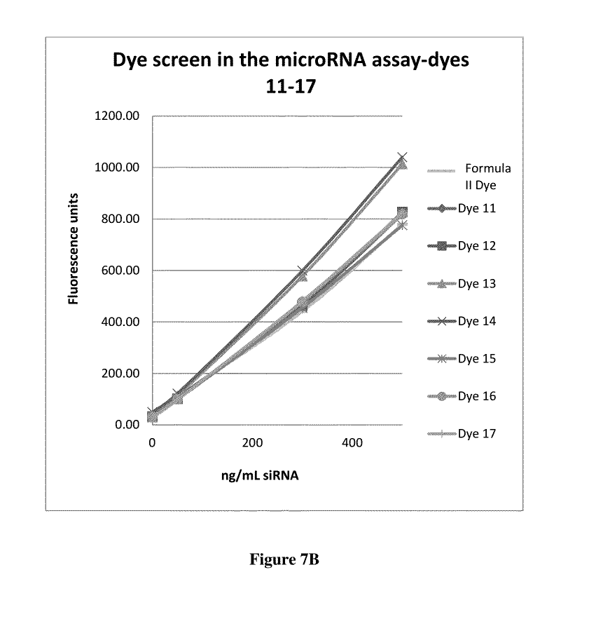

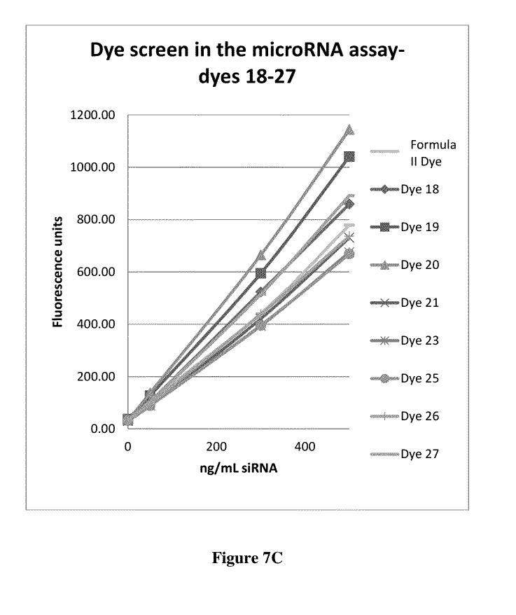

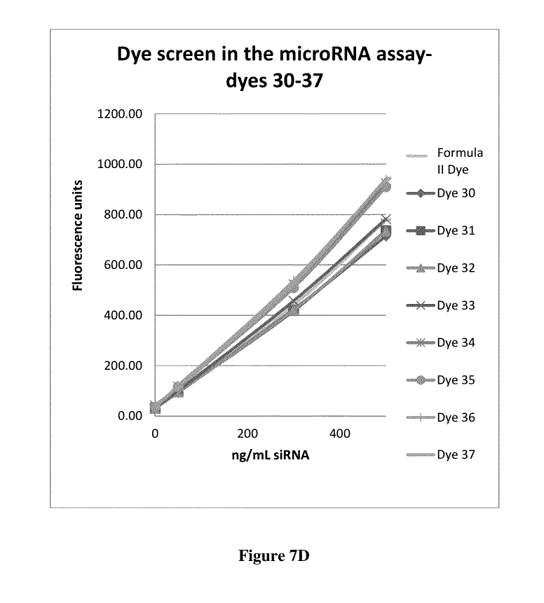

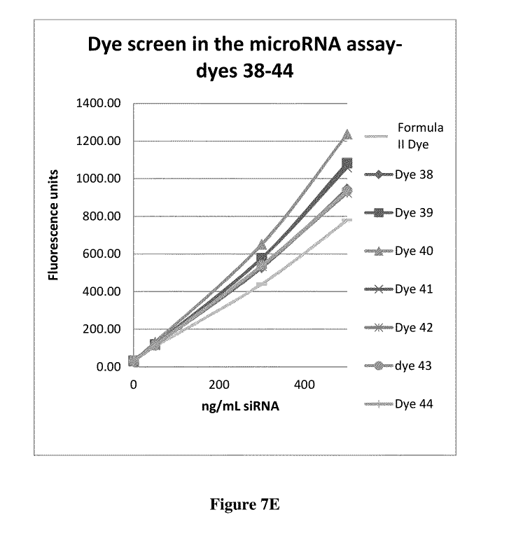

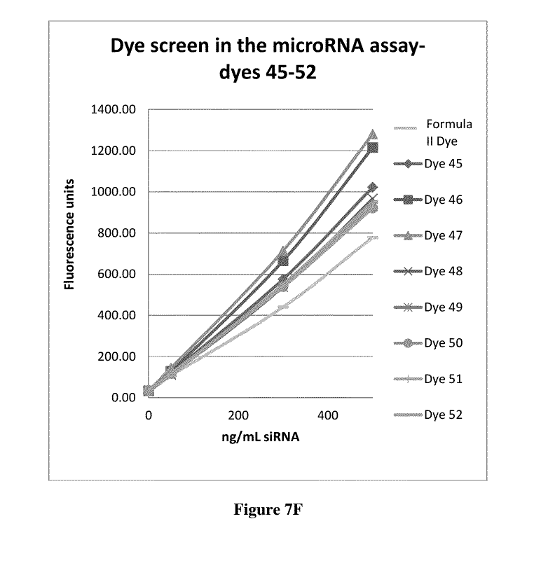

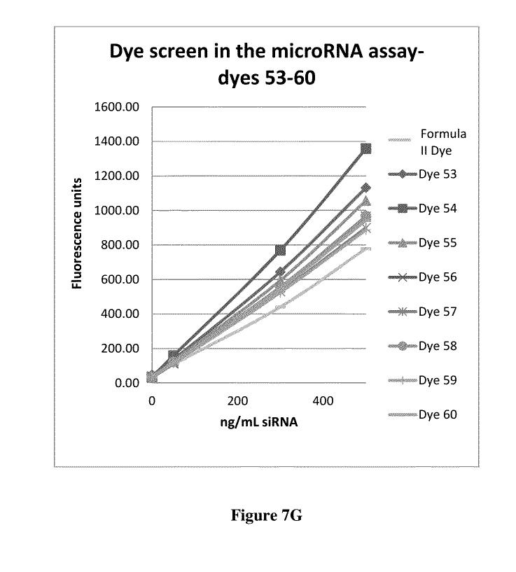

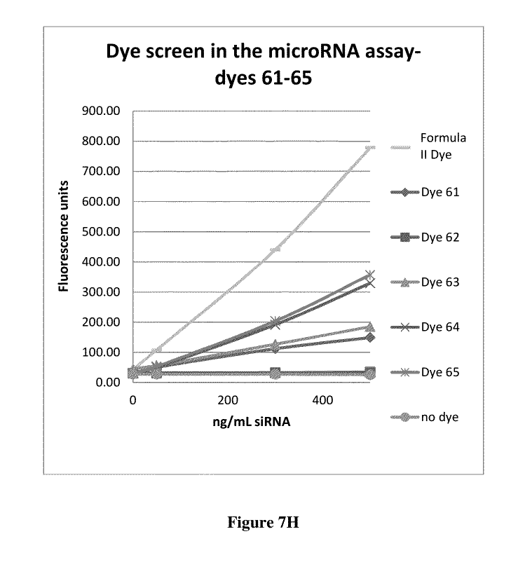

Several of the dye compounds disclosed herein were analyzed for fluorescence enhancement when bound to pure siRNA in the MyQubit miRNA Assay described herein. 58 dye compounds (see Table 5) plus the dye compound of Formula II and a control dye were tested in triplicate. 190 μL of 1 μM dye compound in 1× Tris-EDTA, 0.01% CHAPS was added to 10 μL of 4 different concentrations of siRNA to GAPDH (Ambion cat. #4390849, lot ASO0THWA) in wells of a 96-well Costar black, clear bottom microplate. Plates were read on the SpectraMax plate reader in J2 west at 495 nm excitation and 525 nm emission. FIGS. 7A-H show the fluorescence signal for each of the dyes tested (FIG. 7A=dye compounds 4-10, FIG. 7B=dye compounds 11-17, FIG. 7C=dye compounds 18-27, FIG. 7D=dye compounds 30-37, FIG. 7E=dye compounds 38-44, FIG. 7F=dye compounds 45-52, FIG. 7G=dye compounds 53-60, FIG. 7H=dye compounds 61-65). All of the dye compounds tested had some fluorescence enhancement in the presence of siRNA, except dye compound 62. Dye compounds that had a fluorescence enhancement signal that was >30% greater than that of the dye compound of Formula II at 500 ng/mL siRNA were dye compounds 7, 13, 14, 19, 20, 39, 40, 41, 46, 47, 53, 54, and 55. Dye compounds 61-65 had a low fluorescence signal.

TABLE 5
Dye compounds of Formula III tested in MyQubit miRNA Assay
(Formula III)

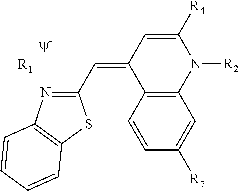

| Compound | R₁ | R₂ | R₄ | R₇ |
|---|---|---|---|---|
| 4 | —CH₃ | phenyl | 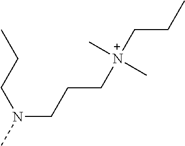 | H |
| 5 | —CH₃ | phenyl | 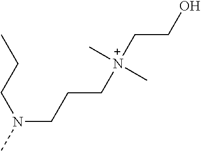 | H |
| 6 | —CH₃ | phenyl | 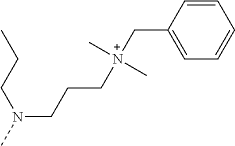 | H |
| 7 | —CH₃ | phenyl | 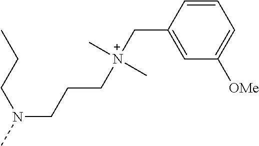 | H |
| 8 | —CH₃ | phenyl | 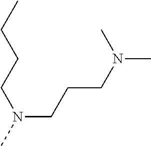 | H |
| 9 | —CH₃ | phenyl | 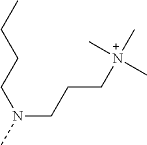 | H |
| 10 | —CH₃ | phenyl | 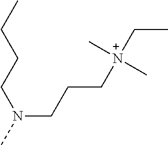 | H |
| 11 | —CH₃ | phenyl | 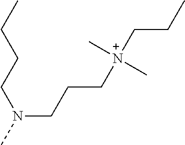 | H |
| 12 | —CH₃ | phenyl | 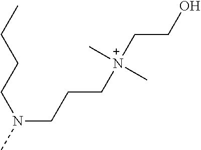 | H |
| 13 | —CH₃ | phenyl | 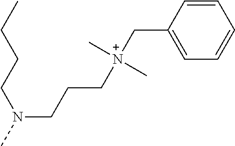 | H |
| 14 | —CH₃ | phenyl | 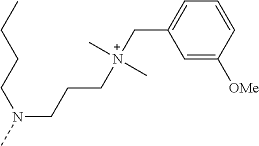 | H |
| 15 | —CH₃ | phenyl | 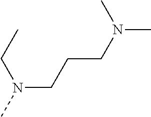 | H |
| 16 | —CH₃ | phenyl | 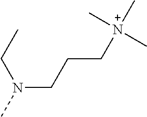 | H |
| 17 | —CH₃ | phenyl | 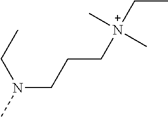 | H |
| 18 | —CH₃ | phenyl | 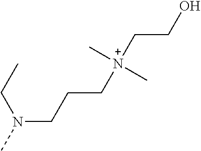 | H |
| 19 | —CH₃ | phenyl | 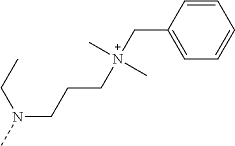 | H |
| 20 | —CH₃ | phenyl | 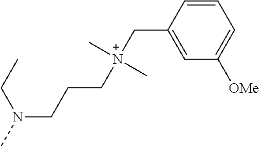 | H |
| 21 | —CH₃ | phenyl | 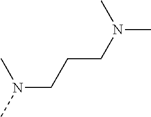 | H |
| 23 | —CH₃ | phenyl | 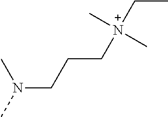 | H |
| 25 | —CH₃ | phenyl | 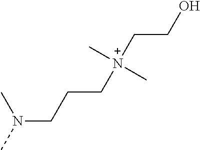 | H |
| 26 | —CH₃ | phenyl | 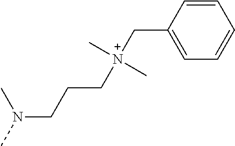 | H |
| 27 | —CH₃ | phenyl | 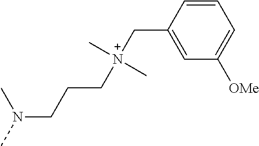 | H |
| 30 | —CH₃ | phenyl | 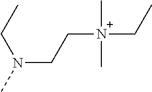 | H |
| 31 | —CH₃ | phenyl | 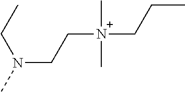 | H |
| 32 | —CH₃ | phenyl | 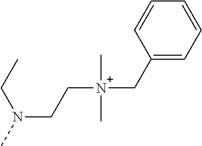 | H |
| 33 | —CH₃ | phenyl | 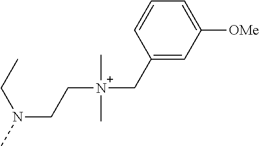 | H |
| 34 | —CH₃ | phenyl | 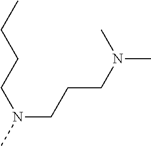 | —OCH₃ |
| 35 | —CH₃ | phenyl | 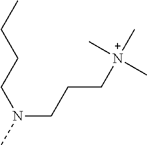 | —OCH₃ |
| 36 | —CH₃ | phenyl | 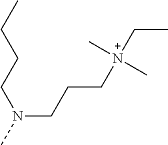 | —OCH₃ |
| 37 | —CH₃ | phenyl | 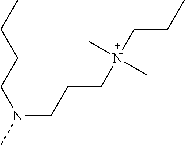 | —OCH₃ |
| 38 | —CH₃ | phenyl | 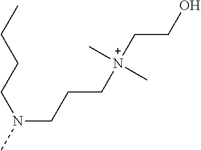 | —OCH₃ |
| 39 | —CH₃ | phenyl | 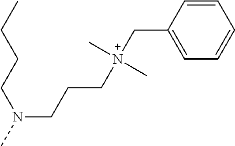 | —OCH₃ |
| 40 | —CH₃ | phenyl | 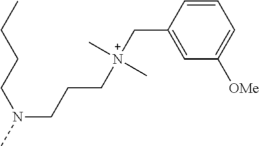 | —OCH₃ |
| 41 | —CH₃ | phenyl | 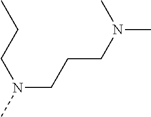 | —OCH₃ |
| 42 | —CH₃ | phenyl | 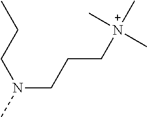 | —OCH₃ |
| 43 | —CH₃ | phenyl | 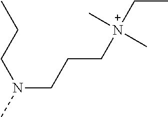 | —OCH₃ |
| 44 | —CH₃ | phenyl | 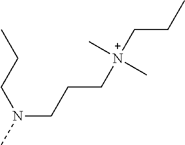 | —OCH₃ |
| 45 | —CH₃ | phenyl | 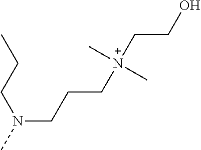 | —OCH₃ |
| 46 | —CH₃ | phenyl | 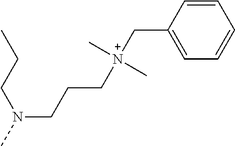 | —OCH₃ |
| 47 | —CH₃ | phenyl | 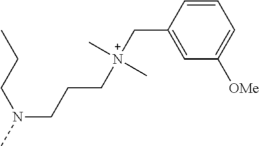 | —OCH₃ |
| 48 | —CH₃ | phenyl | 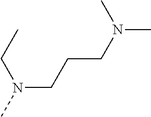 | —OCH₃ |
| 49 | —CH₃ | phenyl | 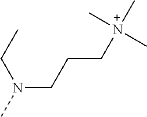 | —OCH₃ |
| 50 | —CH₃ | phenyl | 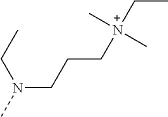 | —OCH₃ |
| 51 | —CH₃ | phenyl | 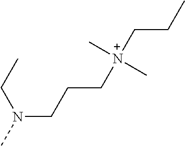 | —OCH₃ |
| 52 | —CH₃ | phenyl | 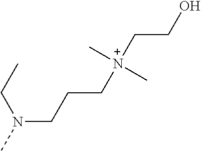 | —OCH₃ |
| 53 | —CH₃ | phenyl | 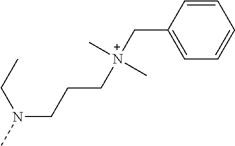 | —OCH₃ |
| 54 | —CH₃ | phenyl | 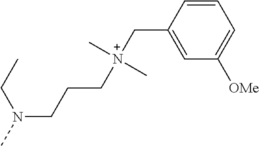 | —OCH₃ |
| 55 | —CH₃ | phenyl | 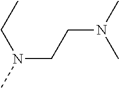 | —OCH₃ |
| 56 | —CH₃ | phenyl | 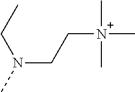 | —OCH₃ |
| 57 | —CH₃ | phenyl | 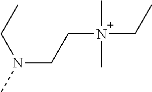 | —OCH₃ |
| 58 | —CH₃ | phenyl | 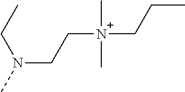 | —OCH₃ |
| 59 | —CH₃ | phenyl | 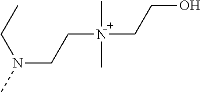 | —OCH₃ |
| 60 | —CH₃ | phenyl | 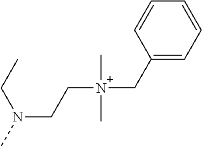 | —OCH₃ |
| 61 | —CH₃ | phenyl | 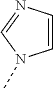 | H |
| 62 | —CH₃ | phenyl | 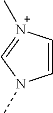 | H |
| 63 | —CH₃ | phenyl | 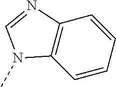 | H |
| 64 | —CH₃ | phenyl | —CH₂CH₂CH₂N(CH₃)₂ | H |
| 65 | —CH₃ | phenyl | —CH₂CH₂CH₂N⁺(CH₃)₃ | H |

The foregoing examples illustrate various aspects of the present teachings and practice of the methods disclosed herein. The examples are not intended to provide an exhaustive description of the many different embodiments of the invention. Thus, although the forgoing invention has been described in some detail by way of illustration and example for purposes of clarity of understanding, those of ordinary skill in the art will realize readily that many changes and modifications can be made thereto without departing from the spirit or scope of the following appended claims.

1. A method of detecting the presence of single-stranded RNA (ssRNA) in a sample, comprising:
(a) combining a sample suspected of containing ssRNA, wherein the ssRNA comprises 25 or fewer nucleotides, with a substituted, unsymmetrical cyanine dye compound;
(b) incubating the sample for a time sufficient for the dye compound to combine with the ssRNA in the sample, if present, to form a dye-ssRNA complex that emits a detectable fluorescent signal; and
(c) detecting for the emission of the fluorescent signal, wherein the detectable fluorescent signal is indicative of the presence of a dye-ssRNA complex.

2-15. (canceled)

16. A method of detecting single-stranded RNA (ssRNA) in a sample, comprising:
(a) combining a sample suspected of containing ssRNA, wherein the ssRNA comprises 25 or fewer nucleotides, with a substituted, unsymmetrical cyanine dye compound of Formula I: 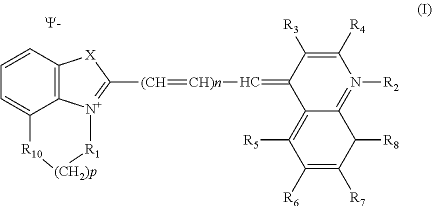
wherein
X is O or S;
n is 0, 1 or 2;
p is 0, 1 or 2;
Ψ is a biologically compatible counterion;
R₁ and R₁₀, which can be the same or different, are independently H; or a substituted or unsubstituted alkyl group, wherein the substitution on the alkyl group, if present, is a carboxyl or (alkyl)ammonium group;
R₂ is a substituted or unsubstituted alkyl, naphthyl, phenyl, thienyl, or cycloalkyl having 3-8 carbons, wherein the substitution on R₂, if present, is a carboxyl or carboxamide group;
R₃ is H; or an alkyl, alkenyl, polyalkenyl, alkynyl or polyalkynyl group; or a halogen; or a substituted or unsubstituted aryl or heteroaryl; or a substituted or unsubstituted cycloalkyl having 3-10 carbons; or —OR₁₁, —SR₁₁, —(NR₉R₁₁); or —OSO₂R₁₉; or a TAIL;
wherein R₉ and R₁₁, which can be the same or different, are independently H; or substituted or unsubstituted alkyl groups; or alkyl groups substituted with one or more alkylammonium groups; or 1-2 alicyclic or aromatic rings; or R₉ and R₁₁ taken in combination are —(CH₂)₄— or —(CH₂)₅— to give a 5 or 6 membered ring; and where R₁₉ is alkyl, or perfluoroalkyl, or aryl;
R₄ is an alkyl, alkenyl, polyalkenyl, alkynyl or polyalkynyl group; or a halogen; or a substituted or unsubstituted aryl or heteroaryl; or a substituted or unsubstituted cycloalkyl having 3-10 carbons; or —OR₁₁, —SR₁₁, —(NR₉R₁₁); or —OSO₂R₁₉; or a TAIL;
R₅, R₆, R₇ and R₈, which can be the same or different, are independently H; or an alkyl, alkenyl, polyalkenyl, alkynyl or polyalkynyl group; or a halogen; or a substituted or unsubstituted aryl or heteroaryl; or a substituted or unsubstituted cycloalkyl having 3-8 carbons; or a TAIL; or —OR₁₁, —SR₁₁, or —(NR₉R₁₁);
TAIL is a heteroatom-containing moiety having the formula LINK-SPACER-CAP;
wherein
LINK is a single covalent bond, —O—, —S—, or —NR₂₀; where R₂₀ is H, a linear or branched alkyl having 1-8 carbons, or R₂₀ is -SPACER-CAP′;
SPACER and SPACER′, which can be the same or different, are covalent linkages, linear or branched, cyclic or heterocyclic, saturated or unsaturated, each having 1-16 nonhydrogen atoms selected from the group consisting of C, N, O and S, such that the linkage begins and ends with a carbon atom, and contains any combination of ether, thioether, amine, ester, amide, or aliphatic, oleic or aromatic carbon-carbon bonds, or aromatic carbon-nitrogen or nitrogen-nitrogen bonds; wherein all heteroatoms in the linear backbone of SPACER are separated by at least two carbon atoms;
CAP and CAP′, which can be the same or different, are —OR₂₁, —SR₂₁, —NR₂₁R₂₂, or —N⁺R₂₁R₂₂R₂₃Ψ⁻;
wherein
R₂₁, R₂₂, and R₂₃ are independently H, or a linear or branched alkyl or cycloalkyl having 1-8 carbons, optionally further substituted by halogen, hydroxy, alkoxy having 1-8 carbons, carboxyalkyl having 1-8 carbons, or phenyl, where phenyl is optionally further substituted by halogen, hydroxy, alkoxy having 1-8 carbons, aminoalkyl having 1-8 carbons, or carboxyalkyl having 1-8 carbons; or, one or more of R₂₁, R₂₂, and R₂₃, taken in combination with SPACER or SPACER′ or R₂₀ forms a 5- or 6-membered aromatic, heteroaromatic, alicyclic or heteroalicyclic ring, the heteroatoms selected from O, N or S; where Ψ⁻ is a biologically compatible counterion; or
CAP and CAP′ are independently 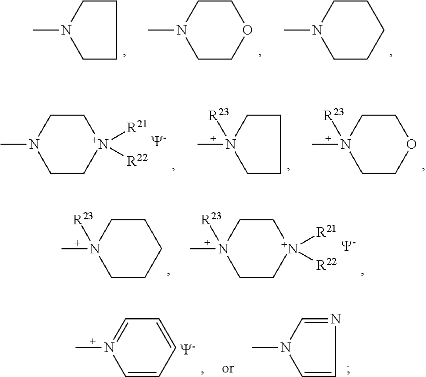
where R₂₁, R₂₂, R₂₃ and Ψ⁻ are as defined previously; such that at least one of R₃, R₄, R₅, R₆, R₇, R₈ or R₁₀ is a TAIL; and, where more than one substituent is a TAIL, each TAIL is optionally the same or different;
or wherein at least one of R₁, R₅, R₆, R₇, R₈, R₉, R₁₀, R₁₁ or R₂₀, R₂₁, R₂₂ or R₂₃ is a water-soluble group (WSG); and
(b) incubating the sample for a time sufficient for the dye compound to combine with the ssRNA in the sample, if present, to form a dye-ssRNA complex that emits a detectable fluorescent signal; and
(c) detecting for the emission of the fluorescent signal, wherein the detectable fluorescent signal is indicative of the presence of a dye-ssRNA complex.

17-23. (canceled)

24. The method of claim 16, wherein the water-soluble group comprises a polymer.

25-26. (canceled)

27. The method of claim 16, wherein if p is 0, and wherein R₁ and R₁₀, which can be the same or different, are independently H; or a substituted or unsubstituted alkyl group, wherein the substitution on the alkyl group, if present, is a carboxyl or (alkyl)ammonium group.

28-29. (canceled)

30. The method of claim 16, wherein if p is not 0, R₁ and R₁₀, which can be the same or different, are independently a substituted or unsubstituted alkyl group, wherein the substitution on the alkyl group, if present, is a carboxyl or (alkyl)ammonium group.

31. The method of claim 16, wherein the dye compound is selected from the group consisting of Compound 4, Compound 5, Compound 6, Compound 7, Compound 8, Compound 9, Compound 10, Compound 11, Compound 12, Compound 13, Compound 14, Compound 15, Compound 16, Compound 17, Compound 18, Compound 19, Compound 20, Compound 21, Compound 23, Compound 25, Compound 26, Compound 27, Compound 30, Compound 31, Compound 32, Compound 33, Compound 34, Compound 35, Compound 36, Compound 37, Compound 38, Compound 39, Compound 40, Compound 41, Compound 42, Compound 43, Compound 44, Compound 45, Compound 46, Compound 47, Compound 48, Compound 49, Compound 50, Compound 51, Compound 52, Compound 53, Compound 54, Compound 55, Compound 56, Compound 57, Compound 58, Compound 59, Compound 60, Compound 61, Compound 62, Compound 63, Compound 64, and Compound 65.

32-36. (canceled)

37. The method of claim 16, wherein the ssRNA is microRNA (miRNA), small nuclear RNA (snRNA), small nucleolar RNA (snoRNA), tiny non-coding RNA (tncRNA), small modulatory RNA (smRNA), or piwi-interacting RNA (piRNA).

38. The method of claim 16, further comprising detecting ssRNA in a concentration of 5 ng/mL or greater.

39. (canceled)

40. The method of claim 16, further comprising detecting ssRNA in a concentration of about 5 ng/mL to about 1000 ng/mL.

41-43. (canceled)

44. The method of claim 16, wherein the fluorescence signal is stable for at least 30 minutes at room temperature.

45. (canceled)

46. A method of detecting single-stranded RNA (ssRNA) in a sample, comprising:
(a) combining a sample suspected of containing ssRNA, wherein the ssRNA comprises 25 or fewer nucleotides, with a substituted, unsymmetrical cyanine dye compound according to claim 16, wherein the compound is of Formula II: 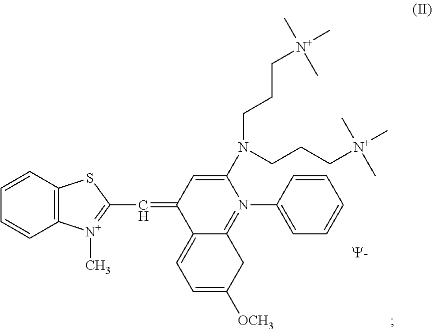
(b) incubating the sample for a time sufficient for the dye compound to combine with the ssRNA in the sample, if present, to form a dye-ssRNA complex that emits a detectable fluorescent signal; and
(c) detecting for the emission of the fluorescent signal, wherein the detectable fluorescent signal is indicative of the presence of a dye-ssRNA complex.

47-60. (canceled)

61. The method of claim 1, wherein
the dye compound comprises a first heterocyclic ring system that is a substituted benzothiazolium, benzoxazolium, benzoselenazolium, benzimidazolium, or dialkylindolinium, wherein the first ring system is linked by a monomethine or trimethine bridging moiety attached at the 2-position of the first ring system to the 2- or 4-position of a second heterocyclic ring system that is a substituted quinolinium or that is linked by a monomethine bridging moiety attached to the 2-position of a second heterocyclic ring system that is a substituted pyridinium; wherein one or more substituents of the second ring system is a substituted or unsubstituted cyclohexyl, cyclohexenyl, morpholino, piperidinyl, naphthyl, phenyl, thienyl, benzothizolyl, furanyl, oxazolyl, benzoxazolyl or pyridinyl group.

62. A fluorescent complex, comprising ssRNA, wherein the ssRNA comprises 25 or fewer nucleotides, non-covalently bound to a dye according to claim 46, wherein the complex emits a detectable fluorescent signal.

63. A fluorescent complex, comprising ssRNA, wherein the ssRNA comprises 25 or fewer nucleotides, non-covalently bound to a dye according to claim 16 wherein the complex emits a detectable fluorescent signal.

64-77. (canceled)

78. The fluorescent complex of claim 63, wherein the dye compound is selected from the group consisting of Compound 4, Compound 5, Compound 6, Compound 7, Compound 8, Compound 9, Compound 10, Compound 11, Compound 12, Compound 13, Compound 14, Compound 15, Compound 16, Compound 17, Compound 18, Compound 19, Compound 20, Compound 21, Compound 23, Compound 25, Compound 26, Compound 27, Compound 30, Compound 31, Compound 32, Compound 33, Compound 34, Compound 35, Compound 36, Compound 37, Compound 38, Compound 39, Compound 40, Compound 41, Compound 42, Compound 43, Compound 44, Compound 45, Compound 46, Compound 47, Compound 48, Compound 49, Compound 50, Compound 51, Compound 52, Compound 53, Compound 54, Compound 55, Compound 56, Compound 57, Compound 58, Compound 59, Compound 60, Compound 61, Compound 62, Compound 63, Compound 64, and Compound 65.

79. (canceled)

80. A kit for detecting the presence of single-stranded RNA (ssRNA) in a sample, comprising:
a dye of the Formula II; and
instructions for using the dye in the method of claim 1.

81. A kit for detecting the presence of single-stranded RNA (ssRNA) in a sample, comprising:
a dye of the Formula I; and
instructions for using the dye in the method of claim 1.

82. A kit for detecting the presence of single-stranded RNA (ssRNA) in a sample, comprising:
a dye compound selected from the group consisting of Compound 4, Compound 5, Compound 6, Compound 7, Compound 8, Compound 9, Compound 10, Compound 11, Compound 12, Compound 13, Compound 14, Compound 15, Compound 16, Compound 17, Compound 18, Compound 19, Compound 20, Compound 21, Compound 23, Compound 25, Compound 26, Compound 27, Compound 30, Compound 31, Compound 32, Compound 33, Compound 34, Compound 35, Compound 36, Compound 37, Compound 38, Compound 39, Compound 40, Compound 41, Compound 42, Compound 43, Compound 44, Compound 45, Compound 46, Compound 47, Compound 48, Compound 49, Compound 50, Compound 51, Compound 52, Compound 53, Compound 54, Compound 55, Compound 56, Compound 57, Compound 58, Compound 59, Compound 60, Compound 61, Compound 62, Compound 63, Compound 64, and Compound 65; and
instructions for using the dye in the method of claim 1.

83. (canceled)

84. A dye compound selected from the group consisting of Compound 4, Compound 5, Compound 6, Compound 7, Compound 8, Compound 9, Compound 10, Compound 11, Compound 12, Compound 13, Compound 14, Compound 15, Compound 16, Compound 17, Compound 18, Compound 19, Compound 20, Compound 21, Compound 23, Compound 25, Compound 26, Compound 27, Compound 30, Compound 31, Compound 32, Compound 33, Compound 34, Compound 35, Compound 36, Compound 37, Compound 38, Compound 39, Compound 40, Compound 41, Compound 42, Compound 43, Compound 44, Compound 45, Compound 46, Compound 47, Compound 48, Compound 49, Compound 50, Compound 51, Compound 52, Compound 53, Compound 54, Compound 55, Compound 56, Compound 57, Compound 58, Compound 59, Compound 60, Compound 61, Compound 62, Compound 63, Compound 64, and Compound 65.
